(19) United States
(12) Patent Application Publication
(10) Pub. No.: US 2026/0078146 A1
(43) Pub. Date: Mar. 19, 2026

(54) EPHA2 TARGETING AGENTS AND USES THEREOF

(71) Applicant: The Regents of the University of California, Oakland, CA (US)

(72) Inventors: Maurizio PELLECCHIA, Riverside, CA (US); Carlo BAGGIO, Riverside, CA (US); Parima UDOMPHOLKUL, Riverside, CA (US)

(73) Assignee: The Regents of the University of California, Oakland, CA (US)

(21) Appl. No.: 19/121,337

(22) PCT Filed: Oct. 17, 2023
(86) PCT No.: PCT/US2023/035312

§ 371 Date: Apr. 15, 2025

Related U.S. Application Data
(60) Provisional application No. 63/416,890, filed on Oct. 17, 2022.

(51) Int. Cl. C07K 7/08; A61K 38/00; A61K 47/55; A61K 47/64; A61P 35/00

(52) U.S. Cl. CPC C07K 7/08; A61K 47/55; A61K 47/64; A61P 35/00; A61K 38/00

(57) ABSTRACT

EphA2 targeting agents developed herein are potent peptide-mimetics with high affinity (Kds 8-20 nanomolar) for the ligand binding domain, called targefrin. Monomeric versions of targefrin act as antagonists while dimeric versions (targefrin-dimer) of the agent cause receptor internalization and degradation via a lysosomal pathway. Hence, targefrin-dimer agents are effective in reducing pro-oncogenic EphA2 levels in cancer cells when used as single agents or in combination with standards of care. Targefrin-dimers can also sensitive cancer cells that developed resistance to EGER or BRAE inhibitors, and potentially other anti-cancer agents. In addition, the dimeric agents can be conjugated with chemotherapy such as paclitaxel to deliver selectively cytotoxic agent to EphA2 expressing cancer cells. Monomeric agents can be linked to chemotherapy via a stable cleavable linker, accumulate the cytotoxic at the tumor, that then would enter the tumor. Novel composition and examples of these applications are reported.

### CROSS REFERENCE TO RELATED APPLICATION

This application claims priority to U.S. Provisional Application No. 63/416,890 filed on 17 Oct. 2022. The entire content of the application referenced above is hereby incorporated by reference herein.

### BACKGROUND OF THE INVENTION

EphA2 is a receptor tyrosine kinase that in its unbound form is pro-oncogenic in a variety of solid tumors. Its tumor promoting activities include increased cell motility and cell migration, increased trans-endothelia migration, increased angiogenesis, suppression of the immune system. In addition, EphA2 expression confers cancer cells resistance to drugs targeting the EGRF inhibitors and to Her2 inhibitors in various solid tumors, and to BRAF inhibitors in melanoma. Hence, can be translated in effective cancer therapeutics.

Currently there is a need for agents that target EphA2. Such agents would be useful as cancer therapeutics either alone or in combination with other anti-cancer agents.

### SUMMARY OF THE INVENTION

Agents that can target EphA2 have been identified. These EphA2 targeting agents are potent peptide-mimetics with high affinity (Kds 8-20 nanomolar) for the ligand binding domain. Monomeric versions of the compounds (compounds of formula (I) wherein R¹² is H) have been found to be antagonists, while dimeric versions of the agent (compounds of formula (I) wherein R¹² is other than H) cause receptor internalization and degradation via a lysosomal pathway. Hence, dimeric agents are effective in reducing pro-oncogenic EphA2 levels in cancer cells. In addition, the agents can be conjugated with other chemotherapy agents (e.g., paclitaxel) to deliver a selectively cytotoxic agent to EphA2 expressing cancer cells.

Certain compounds of the invention are more soluble and 10 folds more active in causing EphA2 internalization and degradation than previously reported agents such as 135H12 (EphA2 agonists and uses thereof, inventors Pellecchia, et al., assigned to University of California, US20210221843A1; PCT WO2019237075A1; Published also in Gambini et al. ACS Chem Biol. 2018 Sep. 21; 13 (9): 2633-2644. doi: 10.1021/acschembio.8b00556). The agents can therefore i) be used to reduce pro-oncogenic EphA2 levels in solid tumors; ii) be conjugated with other chemotherapy agents to deliver cytotoxic agents more selectively to EphA2 expressing cancer cells; and iii) be used to degrade other surface receptors by linking with molecules that are potent for the targeted receptor.

Accordingly, in one embodiment, the invention provides a compound of formula (I):

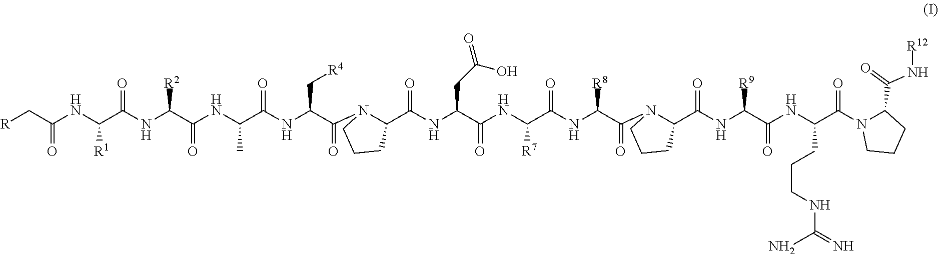

or a salt thereof, wherein:
   each R is independently selected from the group consisting of morpholino, piperidino, and piperazine, that is optionally substituted with (C₁-C₆)alkyl;
   each R¹ is benzyl, 3-indolylmethyl, 4-pyridinylmethyl, 1-naphthylmethyl, or 2-naphthylmethyl, which benzyl, 3-indolylmethyl, 4-pyridinylmethyl, 1-naphthylmethyl, and 2-naphthylmethyl is optionally substituted with one or more groups independently selected from hydroxy, amino, nitro, (C₁-C₆) alkoxy, and (C₁-C₆)alkyl;
   each R² is independently selected from the group consisting of (C₁-C₆)alkyl that is optionally substituted with hydroxy;
   each R⁴ is independently selected from the group consisting of biphenyl and phenoxyphenyl, which biphenyl and phenoxyphenyl is optionally substituted with one or more groups independently selected from halo, hydroxy, (C₁-C₆)alkyl, and (C₁-C₆) alkoxy, wherein each (C₁-C₆)alkyl and (C₁-C₆)alkoxy is optionally substituted with one or more groups independently selected from the group consisting of halo;
   each R⁷ is (C₁-C₆)alkyl that is optionally substituted with hydroxy;
   each R⁸ is independently selected from the group consisting of isopropyl and (C₃-C₆)cycloalkyl;
   each R⁹ is independently selected from the group consisting of benzyl that is optionally substituted with one or more halo;
   R¹² is H or is selected from the group consisting of:

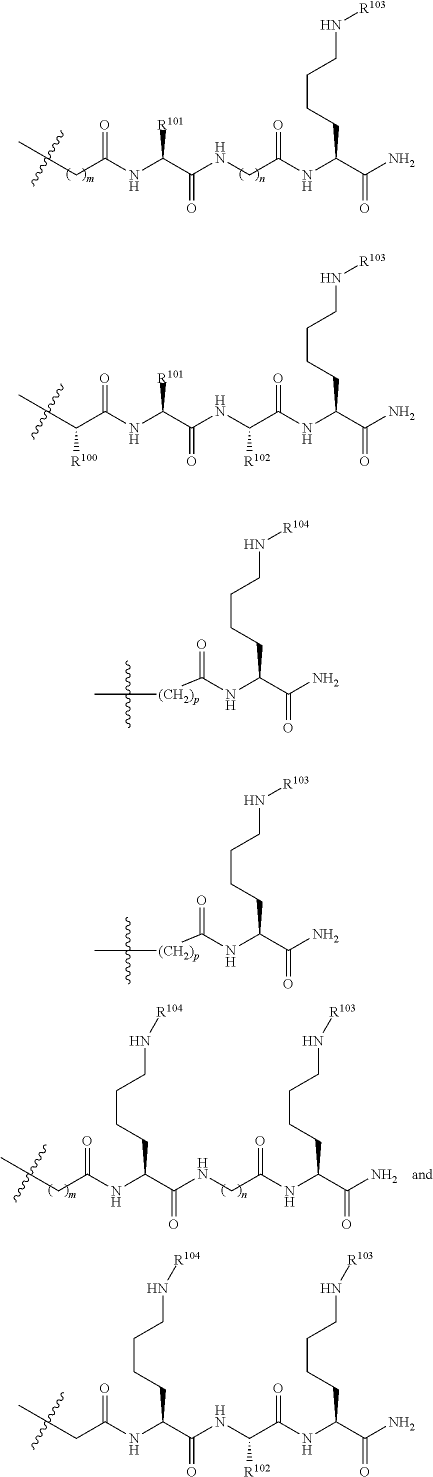

R¹⁰⁰ is H, (C₃-C₆)cycloalkyl, or (C₁-C₆)alkyl that is optionally substituted with hydroxy;
   R¹⁰¹ is H, (C₃-C₆)cycloalkyl, or (C₁-C₆)alkyl that is optionally substituted with hydroxy;
   R¹⁰² is H, (C₃-C₆)cycloalkyl, or (C₁-C₆)alkyl that is optionally substituted with hydroxy;
   R¹⁰³ is -L¹-D;
   D is the residue of a drug or the residue of a targeting agent,
   p is 1, 2, or 3;
   m is 1, 2, or 3;
   n is 1, 2, or 3;
   R¹⁰⁴ is:

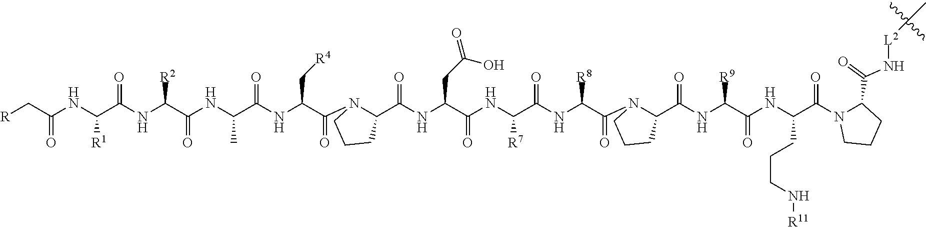

R¹¹ is C(═NH)NH₂;
   L¹ is a linking group; and
   L² is a linking group.

The invention also provides a pharmaceutical composition comprising a compound of formula I or a pharmaceutically acceptable salt thereof, and a pharmaceutically acceptable excipient.

The invention also provides a method for treating or preventing cancer in an animal (e.g., a mammal such as a human) comprising administering a compound of formula I or a pharmaceutically acceptable salt thereof to the animal. In certain embodiments, the cancer is pancreatic cancer, prostate cancer, breast cancer, esophageal cancer, melanoma, urinary bladder, brain cancer, lung cancer, ovarian cancer, stomach cancer, or leukemia. In certain embodiments, the cancer is pancreatic cancer, prostate cancer, breast cancer, or melanoma. In certain embodiments, the cancer is a metastatic cancer.

The invention also provides a compound of formula I or a pharmaceutically acceptable salt thereof for use in medical therapy.

The invention also provides a compound of formula I or a pharmaceutically acceptable salt thereof for the prophylactic or therapeutic treatment of cancer.

The invention also provides the use of a compound of formula I or a pharmaceutically acceptable salt thereof to prepare a medicament for treating cancer in an animal (e.g. a mammal such as a human).

The invention also provides processes and intermediates disclosed herein that are useful for preparing a compound of formula I or a salt thereof.

### BRIEF DESCRIPTION OF THE DRAWINGS

FIGS. 1A-1D show modeling and binding data for targefrin. FIG. 1A) Molecular model of targefrin in complex with EphA2-LBD based on the X-ray structure of the complex with peptide (PDB ID 6B9L). FIG. 1B) Chemical Structure of targefrin. FIG. 1C) Isothermal Titration calorimetry (ITC) curve for the binding between targefrin and EphA2-LBD (K_{d}=21.7±1.2 nM; ΔH=−20.2±0.4 kcal/mol; −TΔS=9.7±0.4 kcal/mol). FIG. 1D) ITC curves for binding between targefrin and EphA4-LBD, and EphA3-LBD, the two Eph receptors with the greatest similarities to EphA2. The data indicated no appreciable binding under these experimental conditions.

FIGS. 2A-2B show data from Example 9. Targefrin functions as an antagonist. (FIG. 2A) Western blot of BxPC3 cells which were starved for 1 h and pre-treated with various concentrations of targefrin for 20 min, followed by a combination treatment with 2 μg/mL ephrinA1-Fc for 3 h. (FIG. 2B) Quantification of the EphA2 level. EphA2/β-actin ratios were normalized by designating the EphA2 expression from DMSO without ephrinA1-Fc condition as 1. EC₅₀ value was calculated to be 1.6±0.1 μM and was presented as mean±standard error (SE) of two independent experiments.

FIGS. 3A-3F show data from Example 11. Targefrin-dimer and its variations cause EphA2 degradation at nanomolar concentrations in pancreatic cancer cell lines. (FIGS. 3A-3C) Western blot images of BxPC3, PANC-1, and MIA PaCa-2 cells, respectively, in which cells were starved for 1 h and treated with 2 μg/mL ephrinA1-Fc or the indicated doses of targefrin and targefrin-dimer and its variations with different linkers (Table 4) for 3 h. Previous dimeric agent 135H121 is also shown as reference. (FIGS. 3D-3F) Densitometry analyses for the data shown in (FIGS. 3A-3C), respectively. EphA2/β-actin ratios were normalized by designating the EphA2 expression from the DMSO control condition as 100% for (FIGS. 3A-3C) or 1 for (FIGS. 3D-3F). ***p<0.001, ****p<0.0001, as determined by a one-way analysis of variance using Dunnett's post-test analysis.

FIGS. 4A-4D show data from Example 12. Chemical structure and biochemical activity of targefrin-conjugated agents. Chemical structures of (FIG. 4A) targefrin conjugated to paclitaxel (targefrin-PTX), (FIG. 4B) dimeric version of targefrin conjugated to paclitaxel (targefrin-dimer-PTX), (FIG. 4C) dimeric version of targefrin conjugated to the 5-carboxytetramethylrhodamine-azie dye (targefrin-dimer-TAMRA). (FIG. 4D) DELFIA displacement dose-response curves comparing targefrin-PTX, targefrin-dimer-PTX, and targefrin-dimer-TAMRA, with their respective IC₅₀ values.

FIG. 5 shows data from Example 13. Targefrin-dimer-TAMRA is internalized in EphA2-expressing cells. BxPC3 cells were treated with 100 nM targefrin-dimer-TAMRA for 0, 30, and 60 min. Upon binding of the agent, EphA2 was internalized and targeted to the lysosomes as shown by colocalizations of 5-TAMRA and LAMP1 (arrowheads). Nuclei were labeled in blue. Scale bar=10 μm.

FIGS. 6A-6C show data from Example 14. Targefrin-dimer significantly inhibits pancreatic cancer cell migration. (FIG. 6A) Cell migration assay of BxPC3 treated with 2 μg/mL ephrinA1-Fc and 10 μM targefrin or the indicated doses of targefrin-dimer. Plates were imaged every 3 h for 24 h. The yellow lines displayed initial scratches made at 0 h, while the black lines displayed the location that the cells had migrated to after 24 h. (FIG. 6B) Targefrin-dimer significantly inhibited cell migration at 24 h in a dose-dependent manner as shown by decreases in relative wound density. Data relative to the 12 h time point are reported as FIG. 10. (FIG. 6C) Time-response curves showed the effects of the agents on wound closure over a period of 24 h. ***p<0.001, ****p<0.0001, as determined by a one-way analysis of variance using Dunnett's post-test analysis. Scale bar=250 μm.

FIGS. 7A-7C show data from Example 15. PTX-conjugated suppressed tumor growth in a tumor xenograft with MIA-PaCa-2 cells. (FIG. 7A) Five groups of 5 mice bearing preestablished MIA-PaCa-2 tumors were treated for 22 days with vehicle control alone, paclitaxel (PTX; 2.5 mg/kg), targefrin-PTX (10 mg/kg which is equivalent to 2.5 mg/kg of PTX), targefrin-dimer-PTX (17 mg/kg which is equivalent to 2.5 mg/kg of PTX), and a lower dose of targefrin-dimer-PTX (10 mg/kg which is equivalent to 1.5 mg/kg of PTX). The tumor volume is reported as average±SE. (FIG. 7B) Average tumor volume for each group of treatment, measured at 0, 8, 15, and 22 days. *p=0.03, **p<0.01, ***p=0.0001, ****p<0.0001, as determined by a two-way analysis of variance using Tukey's post-test analysis. (FIG. 7C) The average body weight±SE is reported for each of the five groups of treatment at 0, 8, 15, and 22 days. For all the graphs, the vehicle is reported in black, PTX in green, targefrin-dimer-PTX at lower dose in light blue, targefrin-dimer-PTX in blue, and targefrin-PTX in red.

FIGS. 8A-8F show Targefrin-dimer-PTX retain its ability to cause EphA2 degradation in pancreatic cancer cell lines. (FIGS. 8A-8C) Western blot images of BxPC3, PANC-1, and MIA PaCa-2 cells, respectively, in which cells were starved for 1 h and treated with 2 μg/mL ephrinA1-Fc or the indicated doses of targefrin, targefrin-PTX, targefrin-dimer, and targefrin-dimer-PTX for 3 h. (FIGS. 8D-8F) Densitometry analyses of (FIGS. 8A-8C), respectively. EphA2/β-actin ratios were normalized by designating the EphA2 expression from the DMSO control condition as 100% for (FIGS. 8A-8C) or 1 for (FIGS. 8D-8F). ***p<0.001, ****p<0.0001, as determined by a one-way analysis of variance using Dunnett's post-test analysis.

FIG. 9. Pharmacokinetics studies. Preliminary pharmacokinetic (PK) studies with Targefrin-dimer. The agent has been injected IV via the tail vein at a concentration of 50 mg/Kg in a formulation of 80% PBS, 10% Tween 80, and 10% Ethanol. Note that is formulation resulted in a clear solution containing 20 mg/ml of targefrin-dimer. Cₘₐₓ˜650 ng/ml after 2 hours from the injection. Estimated t_{1/2}˜15 hr.

FIGS. 10A-10B show cell migration assay of BxPC3 from FIG. 9 at 12 h. FIG. 10A) Cell migration assay of BxPC3 treated with 2 μg/mL ephrinA1-Fc and 10 μM targefrin or the indicated doses of targefrin-dimer. The yellow lines displayed initial scratches made at 0 h while the black lines displayed the location that the cells had migrated to after 12 h. FIG. 10B) Targefrin-dimer significantly inhibited cell migration at 12 h in a dose-dependent manner as shown by decreases in relative wound density. ***p<0.01, ****p<0.0001, as determined by a one-way analysis of variance using Dunnett's post-test analysis.

FIGS. 11A-11D. Structural details relative to optimization process. (FIG. 11A) Structure of EphA2-LBD in complex with an earlier agent developed in our laboratory (1). The surface of the receptor and the D-E, G-H, and J-K loops are shown. (FIG. 11B) Detail of the tyrosine residue in position 4 of the EphA2 binding agent that protrudes into a large hydrophobic pocket located between the D-E and J-K loops. (FIG. 11C) Detail of position 1 of the EphA2 binding agent that substitutes tyrosine 1 and N-terminal amide of the YSA peptide, located between the G-H and J-K loops. (FIG. 11D) Detail of the pair of serine residues in the EphA2 binding agent forming an intramolecular hydrogen bond (see arrow), constraining the peptide in a close conformation in its bound form.

FIGS. 12A-12B. Cell viability assay of MIA PaCa-2 at 72 h. FIG. 12A) MIA PaCa-2 cells were treated with 1 μg/mL ephrinA1-Fc, different doses of Targefrin or Targefrin-dimer for 72 h. Percent confluency was monitored with the IncuCyte S3 live-cell analysis system and percent cell viability was calculated by normalizing the confluency of the treatments to that of the DMSO control. Percent cell viability was not significantly affected across all the treatments, as determined by a two-way analysis of variance using Bonferroni post-test analysis. FIG. 12B) Time-response curves for the percent confluency of MIA PaCa-2 cells after the indicated treatments.

FIGS. 13A-13C. (FIG. 13A) Targefrin-dimer mimics the natural ephrinA1 ligands and cause EphA2 degradation, suppressing metastases. (FIG. 13B) Targefrin-PTX could deliver PTX to cancer cells by virtue of accumulating the toxin on EphA2-expressing metastatic pancreatic cancer cells. This requires specific linker cleavage by extracellular proteins for the toxin to be released and then needs to passively diffuse in tumor cells. (FIG. 13C) When PTX is conjugated to targefrin-dimer, however, EphA2 degradation and pro-metastasis signaling are suppressed. Simultaneously, the toxin is actively transported in EphA2-expressing tumor cells, actively killing primary and metastatic cancer cells.

### DETAILED DESCRIPTION OF THE INVENTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkoxy” refers to an alkyl groups attached to the remainder of the molecule via an oxygen atom (“oxy”).

The term “cycloalkyl” or “carbocycle” refers to a saturated or partially unsaturated (non-aromatic) all carbon ring having 3 to 8 carbon atoms (i.e., (C₃-C₈) carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g. tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms.

Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl) ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4th edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “residue” as it applies to the residue of a compound refers to a compound that has been modified in any manner which results in the creation of an open valence wherein the site of the open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine,) followed by the removal of 1 or more atoms from the second functional group to create the open valence.

In one embodiment, the invention provides a compound of formula (I):

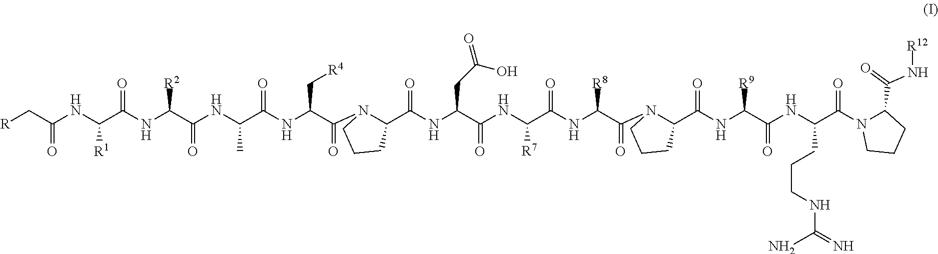

or a salt thereof, wherein:
   each R is independently selected from the group consisting of morpholino, piperidino, and piperazine, that is optionally substituted with (C₁-C₆)alkyl;
   each R¹ is benzyl, 1-naphthylmethyl, or 2-naphthylmethyl, which benzyl, 1-naphthylmethyl, and 2-naphthylmethyl is optionally substituted with one or more groups independently selected from hydroxy, amino, nitro, and (C₁-C₆)alkyl;
   each R² is independently selected from the group consisting of (C₁-C₆)alkyl that is optionally substituted with hydroxy;
   each R⁴ is independently selected from the group consisting of biphenyl and phenoxyphenyl, which biphenyl and phenoxyphenyl is optionally substituted with one or more groups independently selected from halo, hydroxy, (C₁-C₆)alkyl, and (C₁-C₆) alkoxy, wherein each (C₁-C₆)alkyl and (C₁-C₆)alkoxy is optionally substituted with one or more groups independently selected from the group consisting of halo;
   each R⁷ is (C₁-C₆)alkyl that is optionally substituted with hydroxy;
   each R⁸ is independently selected from the group consisting of isopropyl and (C₃-C₆)cycloalkyl;
   each R⁹ is independently selected from the group consisting of benzyl that is optionally substituted with one or more halo;
   R¹² is H or is selected from the group consisting of:

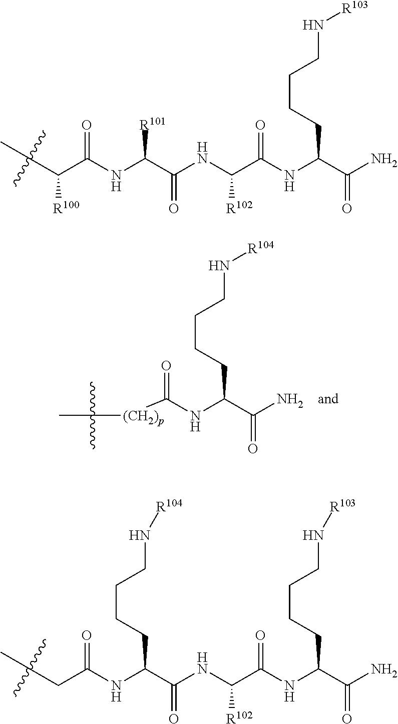

R¹⁰⁰ is H, (C₁-C₂)alkyl or (C₃-C₆)cycloalkyl that is optionally substituted with hydroxy;
   R¹⁰¹ is H, (C₁-C₂)alkyl or (C₃-C₆)cycloalkyl that is optionally substituted with hydroxy;
   R¹⁰² is H, (C₁-C₂)alkyl or (C₃-C₆)cycloalkyl that is optionally substituted with hydroxy;
   R¹⁰³ is -L¹-D;
   D is the residue of a drug or the residue of a targeting agent;
   p is 1, 2, or 3;
   R¹⁰⁴ is:

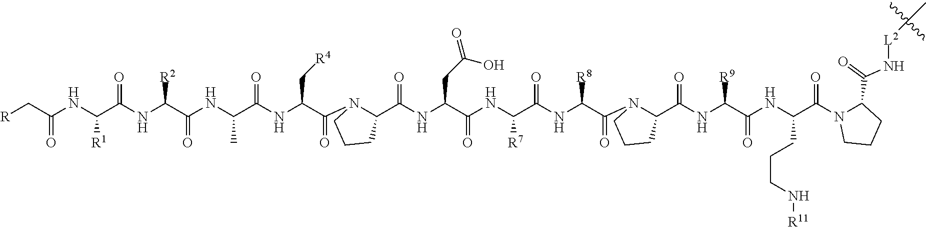

R¹¹ is C(═NH)NH₂;
   L¹ is a linking group; and
   L² is a linking group.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; and aryl can be phenyl, indenyl, or naphthyl.

A specific value for R is morpholino.

A specific value for R is piperidino.

A specific value for R is piperazinyl (e.g., 1-piperazinyl), that is optionally substituted with (C₁-C₆)alkyl.

A specific value for R is 1-piperazyl.

A specific value for R¹ is benzyl that is optionally substituted with amino.

A specific value for R¹ is benzyl that is optionally substituted with nitro.

A specific value for R¹ is benzyl that is optionally substituted with (C₁-C₆)alkyl.

A specific value for R¹ is benzyl that is optionally substituted with hydroxyl.

A specific value for R¹ is benzyl that is optionally substituted with (C₁-C₆)alkoxyl.

A specific value for R¹ is 2-nitrobenzyl, 4-methylbenzyl, or 4-aminobenzyl.

A specific value for R¹ is 4-hydroxylbenzyl, or 4-methoxybenzyl.

A specific value for R¹ is 3-indolylmethyl, 4-pyridinylmethyl, 1-naphthylmethyl, or 2-naphthylmethyl.

A specific value for R¹ is 3-indolylmethyl that is optionally substituted with hydroxyl.

A specific value for R² is selected from the group consisting of (C₁-C₄)alkyl that is optionally substituted with hydroxy.

A specific value for R² is isobutyl or hydroxymethyl.

A specific value for R² is hydroxyethyl (e.g., 1-hydroxyethyl).

A specific value for R⁴ is biphenyl that is optionally substituted with one or more groups independently selected from halo, hydroxy, (C₁-C₆)alkyl, and (C₁-C₆) alkoxy, wherein each (C₁-C₆)alkyl and (C₁-C₆)alkoxy is optionally substituted with one or more groups independently selected from the group consisting of halo.

A specific value for R⁴ is phenoxyphenyl that is optionally substituted with one or more groups independently selected from the group consisting of halo, hydroxy, (C₁-C₆)alkyl, and (C₁-C₆) alkoxy, wherein each (C₁-C₆)alkyl and (C₁-C₆)alkoxy is optionally substituted with one or more groups independently selected from the group consisting of halo.

A specific value for R⁴ is selected from the group consisting of biphenyl, 2′-trifluoromethylbiphenyl, 2′-methylbiphenyl, 4′-chlorobiphenyl, 2′-methoxybiphenyl, 3′-methylbiphenyl, 2′-methyl-4′-methoxybiphenyl, phenoxyphenyl, and 4-(4-hydroxyphenyloxy)phenyl.

A specific value for R⁷ is methyl that is optionally substituted with hydroxyl.

A specific value for R⁷ is methyl.

A specific value for R⁸ is selected from the group consisting of isopropyl and (C₃-C₆)cycloalkyl.

A specific value for R⁸ is cyclohexyl.

A specific value for R⁹ is benzyl.

A specific value for R¹² is H.

A specific value for R¹² is:

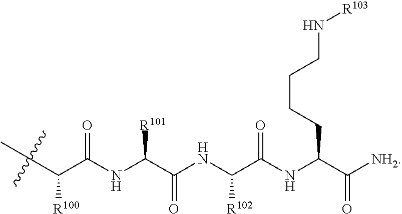

A specific value for R¹² is:

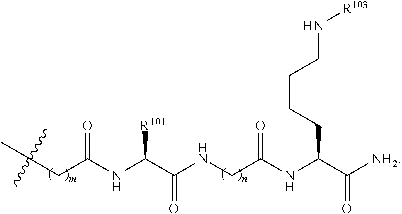

A specific value for m is 1. A specific value for m is 2. A specific value for m is 3.

A specific value for n is 1. A specific value for n is 2. A specific value for n is 3.

A specific value for R¹⁰⁰ is H, (C₃-C₆)cycloalkyl, or (C₁-C₃)alkyl.

A specific value for R¹⁰¹ is H, (C₃-C₆)cycloalkyl, or (C₁-C₃)alkyl.

A specific value for R¹⁰² is H, (C₃-C₆)cycloalkyl, or (C₁-C₃)alkyl.

A specific value for R¹⁰⁰ is H, (C₃-C₆)cycloalkyl, or (C₁-C₂)alkyl.

A specific value for R¹⁰¹ is H, (C₃-C₆)cycloalkyl, or (C₁-C₂)alkyl.

A specific value for R¹⁰² is H, (C₃-C₆)cycloalkyl, or (C₁-C₂)alkyl.

A specific value for R¹⁰⁰ is H, methyl, ethyl, isopropyl, cyclohexyl, or hydroxymethyl.

A specific value for R¹⁰¹ is H, methyl, ethyl, isopropyl, cyclohexyl, or hydroxymethyl.

A specific value for R¹⁰² is H, methyl, ethyl, isopropyl, cyclohexyl, or hydroxymethyl.

A specific value for R¹² is:

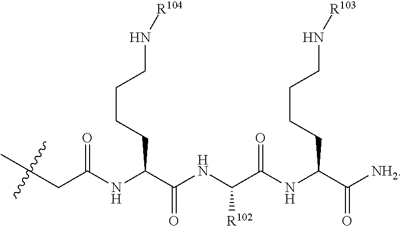

A specific value for R¹² is:

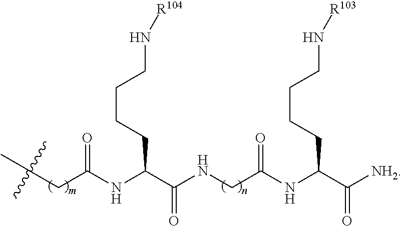

A specific value for m is 1. A specific value for m is 2. A specific value for m is 3.

A specific value for n is 1. A specific value for n is 2. A specific value for n is 3.

A specific value for L¹ is a branched or unbranched, saturated or unsaturated, hydrocarbon chain, having from about 1 to 100 carbon atoms, wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆) alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

A specific value for L¹ is a branched or unbranched, saturated or unsaturated, hydrocarbon chain, having from about 1 to 20 carbon atoms, wherein one or more of the carbon atoms is optionally replaced independently by —O—, —S, —N(R^{a})—, 3-7 membered heterocycle, 5-6-membered heteroaryl or carbocycle and wherein each carbon atom, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆) alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (—O), and carboxy, wherein each R^{a} is independently H or (C₁-C₆)alkyl.

A specific value for L′ is a branched or unbranched, saturated hydrocarbon chain, having from about 5 to 15 carbon atoms, wherein one or more of the carbon atoms is optionally replaced independently by O, NH, or a divalent triazine ring, wherein each carbon atom is optionally and independently substituted with one or more (e.g. 1, 2, 3, 4, 5 or more) substituents selected from halo and oxo (—O).

A specific value for L¹ is:

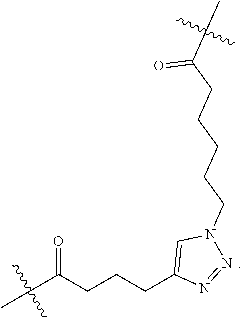 [SMILES: *C(=O)CCCCCn1cc(CCCC(*)=O)nn1]

A specific value for D is a residue of a drug.

A specific value for D is a residue of an anti-cancer agent, including a classical chemotherapeutic agents, such as an antimetabolites (e.g., gemcitabine), antimitotics (e.g., a taxane or DM1, or MMAE), alkylating agents (e.g., chlorambucil), DNA damaging agents (e.g., doxorubicin), and targeted therapeutics (e.g., a kinase inhibitor such as erlotinib). In one embodiment, D is the residue of an EGRF inhibitors (e.g., cetuximab, gefitinib, erlotinib), a Her2 inhibitor (e.g., trastuzumab), a BRAF inhibitor (e.g., vemurafenib, dabrafenib), gemcitabine, 5FU, or another antimetabolite, taxane, alkylating agent, or DNA damaging agent.

In certain embodiments, the antimetabolite is gemcitabine. In certain embodiments, the antimitotics is a taxane or DM1, or MMAE. In certain embodiments, the alkylating agent is chlorambucil. In certain embodiments, the DNA damaging agent is doxorubicin. In certain embodiments, the targeted therapeutics is a kinase inhibitor, such as erlotinib. In one embodiment, D is the residue of an EGRF inhibitor that is cetuximab, gefitinib, or erlotinib. In one embodiment, D is the residue of a Her2 inhibitor that is trastuzumab. In one embodiment, D is the residue of a BRAF inhibitor that is vemurafenib or dabrafenib. In one embodiment, D is the residue of gemcitabine, 5FU, or another antimetabolite, taxane, alkylating agent, or DNA damaging agent.

A specific value for D is a residue of a taxane, including paclitaxel, docetaxel, cabazitaxel.

A specific value for D is a residue of paclitaxel.

A specific value for D is a residue of a targeting agent.

A specific value for R¹² is:

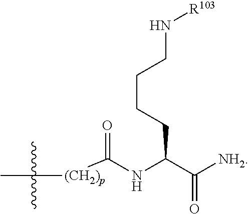

A specific value for R¹² is:

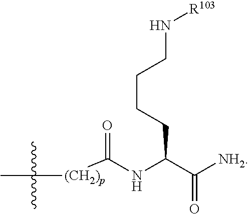

A specific value for p is 1. A specific value for p is 2. A specific value for p is 3.

A specific value for L² is a branched or unbranched, saturated or unsaturated, hydrocarbon chain, having from about 1 to 100 carbon atoms, wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆) alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

A specific value for L² is a branched or unbranched, saturated or unsaturated, hydrocarbon chain, having from about 1 to 20 carbon atoms, wherein one or more of the carbon atoms is optionally replaced independently by —O—, —S, —N(R^{a})—, 3-7 membered heterocycle, 5-6-membered heteroaryl or carbocycle and wherein each carbon atom, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆) alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), and carboxy, wherein each R^{a} is independently H or (C₁-C₆)alkyl.

A specific value for L² is a branched or unbranched, saturated hydrocarbon chain, having from about 3 to 110 carbon atoms (e.g., 3 to 10 carbon atoms), wherein each carbon atom is optionally and independently substituted with one or more (e.g. 1, 2, 3, 4, 5 or more) substituents selected from halo and oxo (═O).

A specific value for L² is —CH₂C(═O)—, —CH₂CH₂C(═O)—, —CH₂CH₂CH₂C(═O)—, —CH₂CH₂CH₂CH₂C(═O)—, or —CH₂CH₂CH₂CH₂CH₂C(═O)—.

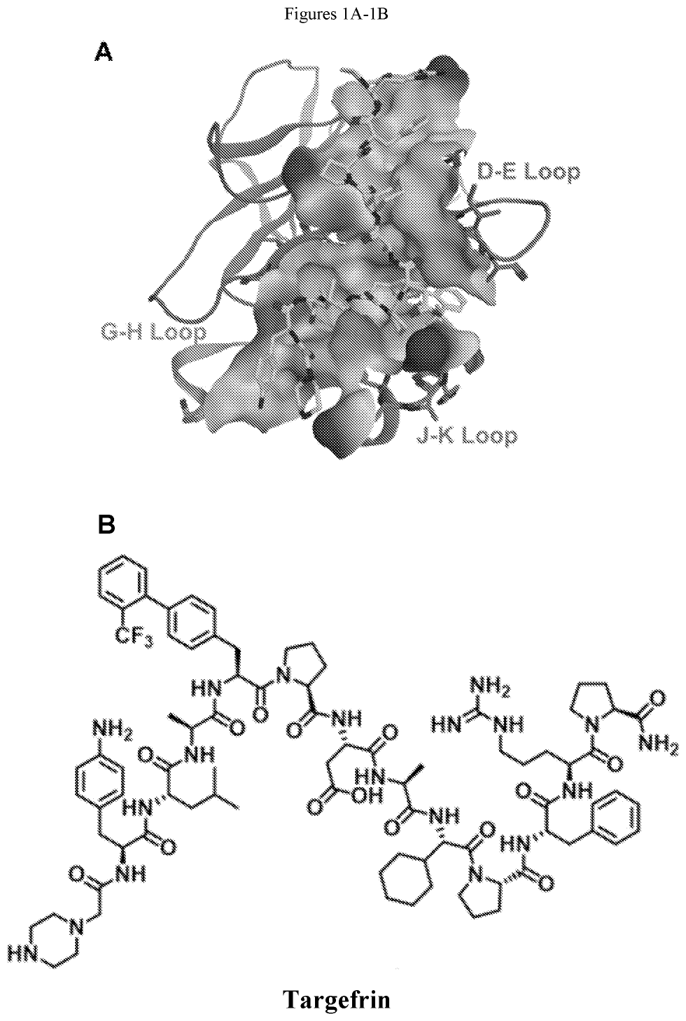

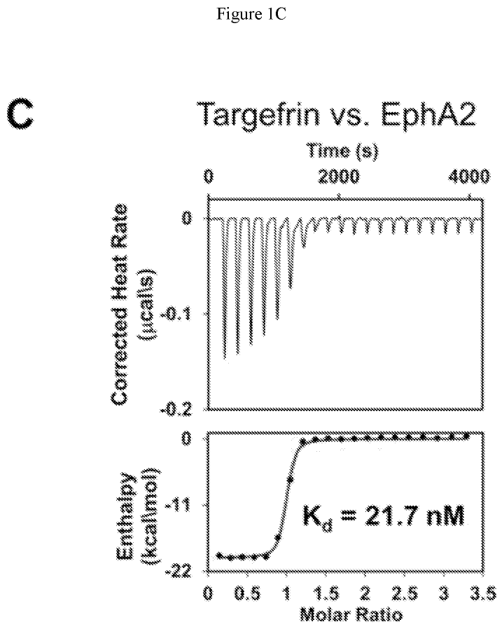

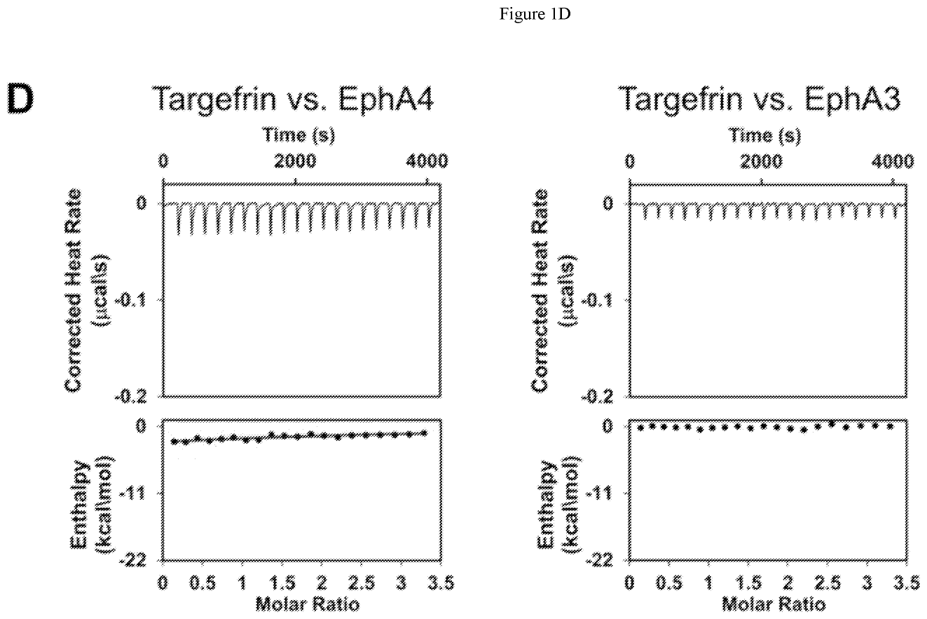

A specific compound or salt is selected from the group consisting of Targefrin (FIG. 1) or Targefrin-conjugated with paclitaxel, or targefrin-dimer, or targefrin-dimer conjugated to paclitaxel:

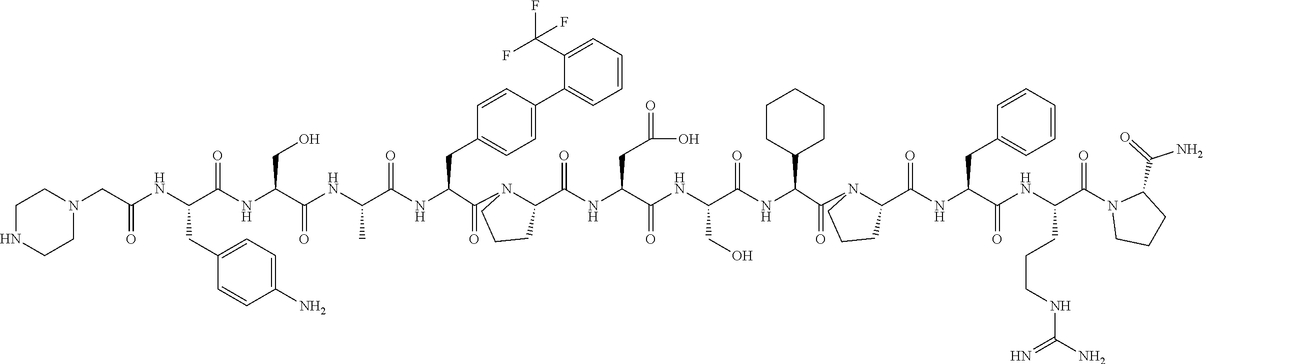 [SMILES: C[C@H](NC(=O)[C@H](CO)NC(=O)[C@H](Cc1ccc(N)cc1)NC(=O)CN1CCNCC1)C(=O)N[C@@H](Cc1ccc(-c2ccccc2C(F)(F)F)cc1)C(=O)N1CCC[C@H]1C(=O)N[C@@H](CC(=O)O)C(=O)N[C@@H](CO)C(=O)N[C@H](C(=O)N1CCC[C@H]1C(=O)N[C@@H](Cc1ccccc1)C(=O)N[C@@H](CCCNC(=N)N)C(=O)N1CCC[C@H]1C(N)=O)C1CCCCC1]

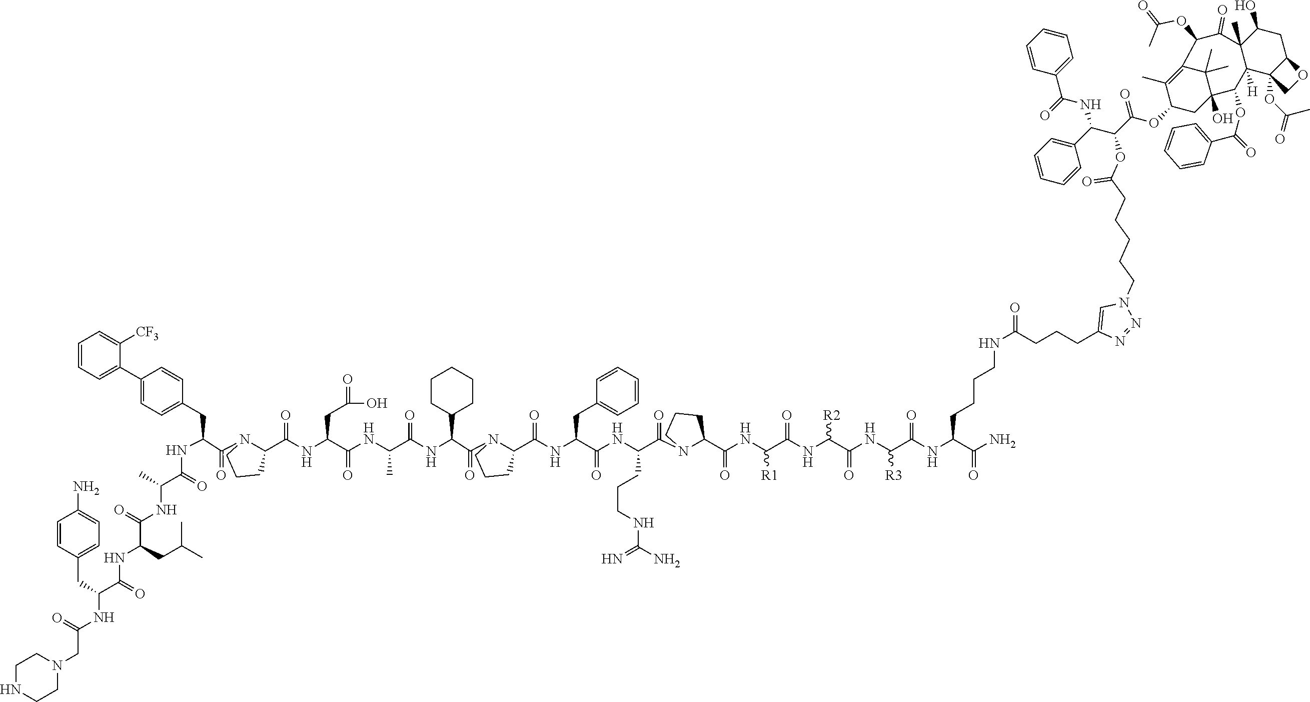

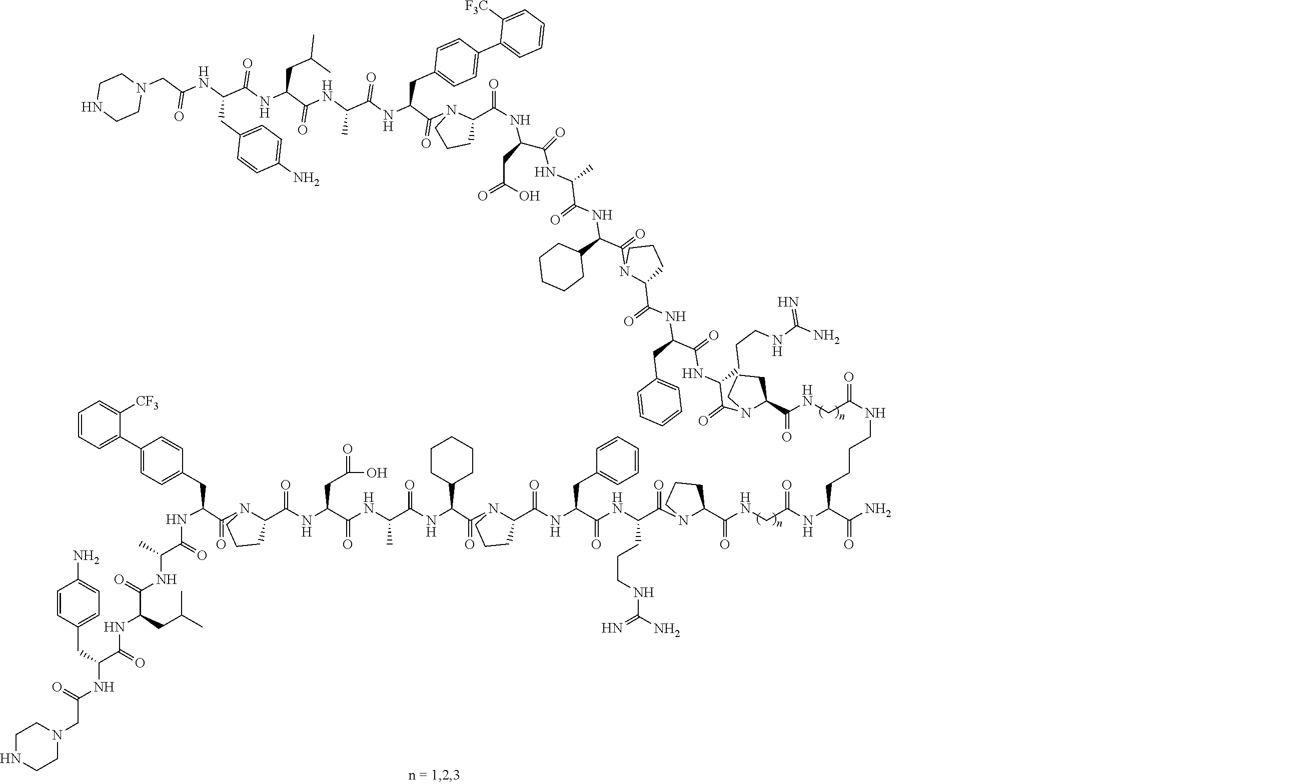

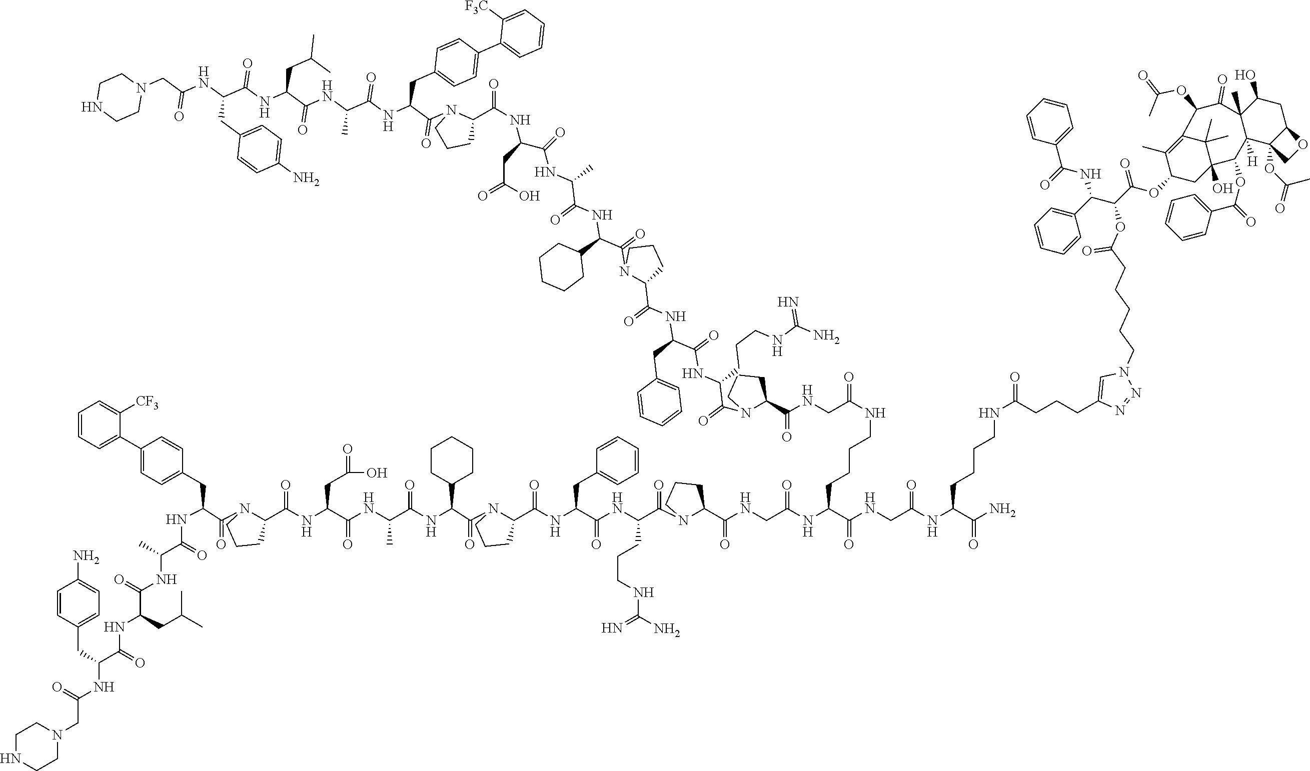

and salts thereof.

In certain embodiments, a compound of formula (I) or a specific compound described herein is a homodimer compound (e.g., targefrin-dimer).

Processes for preparing compounds of formula I are provided as further embodiments of the invention.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical or sub-cutaneous administration, the present compounds may administer as compositions or formulations, in combination with a dermatologically acceptable carrier. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of cancer. Examples of such agents include EGRF inhibitors (i.e. cetuximab, gefitinib, erlotinib), Her2 inhibitors (i.e. trastuzumab), or BRAF inhibitors (vemurafenib, dabrafenib), gemcitabine, 5FU and other classical chemotherapeutic agents, including other antimetabolites, taxanes, alkylating agents, and DNA damaging agents. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat cancer.

Linking Groups L¹, and L²

The compounds of formula (I) may comprise linking groups L¹ and L². In one embodiment the linking group is absent. The linking group can vary in length and atom composition and for example can be branched or non-branched or cyclic or a combination thereof. The linking group may also modulate the properties of the final compound of formula (I), for example, solubility, stability or aggregation.

In one embodiment the linker comprises about 3-100 atoms. In one embodiment the linker comprises about 3-90 atoms. In one embodiment the linker comprises about 3-80 atoms. In one embodiment the linker comprises about 3-70 atoms. In one embodiment the linker comprises about 3-60 atoms. In one embodiment the linker comprises about 3-50 atoms. In one embodiment the linker comprises about 3-400 atoms. In one embodiment the linker comprises about 3-30 atoms. In one embodiment the linker comprises about 3-20 atoms. In one embodiment the linker comprises about 3-10 atoms.

In one embodiment the linker comprises atoms selected from H, C, N, S and O.

In one embodiment the linker comprises atoms selected from H, C, N, and O.

In one embodiment the linker comprises a branched or unbranched, saturated or unsaturated, hydrocarbon chain, having from about 1 to 100 (1-90, 1-80, 1-70, 1-60, 1-50, 1-40, 1-30, 1-20, or 1-10) carbon atoms, wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆) alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

In one embodiment the linker comprises a branched or unbranched, saturated or unsaturated, hydrocarbon chain, having from about 1 to 20 carbon atoms, wherein one or more of the carbon atoms is optionally replaced independently by —O—, —S, —N(R^{a})—, 3-7 membered heterocycle, 5-6-membered heteroaryl or carbocycle and wherein each carbon atom, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆) alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (—O), and carboxy, wherein each R^{a} is independently H or (C₁-C₆)alkyl.

The invention will now be illustrated by the following non-limiting Examples.

### EXAMPLES

### Example 1. Chemical Composition and Mass-Spectrometry Data for Representative Compounds

The compounds shown in the following table 1 were prepared using procedures similar to those described herein or using methods and materials that are known and available.

TABLE 1

| | | Obs. |
|---|---|---|
| | | [M + H]⁺ |
| Sequence | Calcd [M] | (m/z) |
| PiperazAcAcid-(2Nal)-L-A-(2-CF₃-Bip)-PDS- | 1784.8457 | 1785.8530 |
| Chg-P-(4Cl-Phe)-RP-CONH₂ | | |
| PiperazAcAcid-(2NH₂-Phe)-SA-(2-CF₃-Bip)- | 1689.8279 | 1690.8374 |
| PDS-Chg-PFRP-CONH₂ | | |
| PiperazAcAcid-(2NH₂-Phe)-L-A-(2-CF₃-Bip)- | 1715.8800 | (z = 3) |
| PDS-Chg-PFRP-CONH₂ | | 573.3014 |
| | | (z = 2) |
| | | 858.9478 |
| PiperazAcAcid-(2NH₂-Phe)-L-A-(2-CF₃-Bip)- | 1699.8850 | (z = 3) |
| PD-A-Chg-PFRP-CONH₂ | | 567.9696 |
| | | (z = 2) |
| | | 851.4519 |
| [PiperazAcAcid-(2-NH₂-Phe)-L-A-(2-CF₃-Bip)- | 3625.8848 | (z = 5) |
| PD-A-Chg-PFRPG]₂-K-CONH₂ | | 726.3855 |
| | | (z = 4) |
| | | 907.7288 |
| | | (z = 3) |
| | | 1209.9690 |
| [PiperazAcAcid-(2-NH₂-Phe)-L-A-(2-CF₃-Bip)- | 3653.9161 | (z = 5) |
| PD-A-Chg-PFRP-(β-Ala)]₂-K-CONH₂ | | 731.9890 |
| | | (z = 4) |
| | | 914.7353 |
| | | (z = 3) |
| | | 1219.3109 |
| [PiperazAcAcid-(2-NH₂-Phe)-L-A-(2-CF₃-Bip)- | 3681.9474 | (z = 5) |
| PD-A-Chg-PFRP-(GABA)]₂-K-CONH₂ | | 737.5954 |
| | | (z = 4) |
| | | 921.7432 |
| | | (z = 3) |
| | | 1228.6543 |
| PiperazAcAcid-(2-NH₂-Phe)-L-A-(2-CF₃-Bip)- | 2972.4522 | (z = 4) |
| PD-A-Chg-PFRPG-K-(Linker)-PTX | | 744.1197 |
| | | (z = 3) |
| | | 991.8244 |
| | | (z = 2) |
| | | 1487.2337 |
| [PiperazAcAcid-(2-NH₂-Phe)-L-A-(2-CF₃-Bip)- | 4898.4519 | (z = 6) |
| PD-A-Chg-PFRPG]₂-KGK-(Linker)-PTX | | 817.5799 |
| | | (z = 5) |
| | | 980.8969 |
| | | (z = 4) |
| | | 1225.6179 |
| | | (z = 3) |
| | | 1633.8200 |
| [PiperazAcAcid-(2-NH₂-Phe)-L-A-(2-CF₃-Bip)- | 4461.3179 | (z = 7) |
| PD-A-Chg-PFRPG]₂-KGK-(Linker)-TAMRA | | 638.3418 |
| | | (z = 6) |
| | | 744.3911 |
| | | (z = 5) |
| | | 893.0693 |
| | | (z = 4) |
| | | 1116.0822 |
| PiperazineAcAcid-YSA-Bip-PDS-Chg-PFRP- | 1622.8245 | 1623.8279 |
| CONH₂ | | |
| PiperazAcAcid-YSA-(Bip)-PDS-Chg-P-(4Cl- | 1656.7856 | 1657.7900 |
| Phe)-RP-CONH₂ | | |
| PiperazAcAcid-Y-L-A-(Bip)-PDS-Chg-PFRP- | 1648.8766 | 1649.8886 |
| CONH₂ | | |
| PiperazAcAcid-(2Nal)-SA-(Bip)-PDS-Chg- | 1656.8453 | 1657.8520 |
| PFRP-CONH₂ | | |
| PiperazineAcAcid-YSA-(2Me-Bip)-PDS-Chg- | 1636.8402 | 1637.8480 |
| PFRP-CONH₂ | | |
| PiperazAcAcid-(2Nal)-L-A-(2Me-Bip)-PDS- | 1730.8740 | (z = 2) |
| Chg-P-(4Cl-Phe)-RP-CONH₂ | | 866.9478 |
| PiperazAcAcid-Y-L-A-(2Me-Bip)-PDS-Chg-P- | 1696.8533 | 1697.8605 |
| (4Cl-Phe)-RP-CONH₂ | | |

All compounds were analyzed using an Agilent 6545 QTOF LC/MS instrument.

### Example 2

Chemical structures and dissociation constants for EphA2 binding agents are shown. For each compound the Kd value obtained by ITC and the IC₅₀ value resulted from DELFIA 5 displacement measurements are reported against EphA2 ligand binding domain (LBD).

TABLE 2
Chemical structures and dissociation constants for EphA2 binding agents
containing optimal substituents from previous SAR. For each compound we reported the Kd value
obtained by ITC, and the IC₅₀ value resulted from DELFIA displacement measurements.

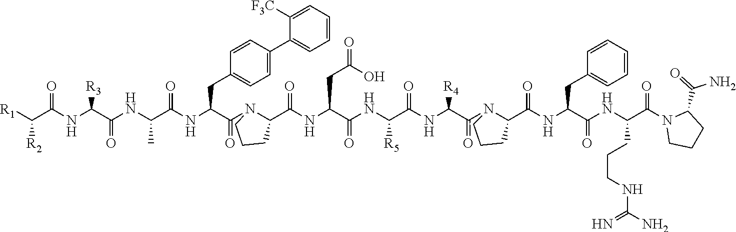

| | | | | | | K_{d} | |
|---|---|---|---|---|---|---|---|
| | | | | | | (nM)- | IC₅₀ |
| | | | | | | ITC | (nM) |
| | | | | | | (EphA2- | (EphA2- |
| ID | R₁ | R₂ | R₃ | R₄ | R₅ | LBD) | LBD) |
| 24 | 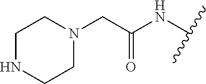 [SMILES: *NC(=O)CN1CCNCC1] | 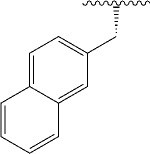 | 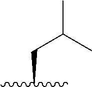 | 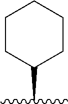 | 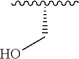 | ND | 15 |
| 25 | 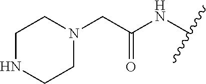 [SMILES: *NC(=O)CN1CCNCC1] | 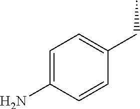 | 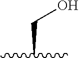 | 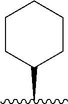 | 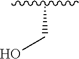 | 18 | 21 |
| 26 | 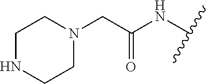 [SMILES: *NC(=O)CN1CCNCC1] | 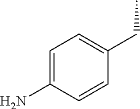 | 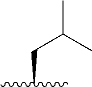 | 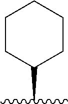 | 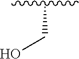 | 24 | 9.3 |
| 27 (Tar- gefrin) | 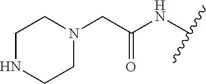 [SMILES: *NC(=O)CN1CCNCC1] | 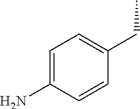 | 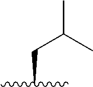 | 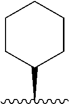 | CH₃ | 21.7 ± 1.2 (n = 2) | 10.8 ± 0.8 |

### Example 3. Synthetic Scheme for the Synthesis of Targefrin

Conditions: (a) Rink Amide resin+3 equiv. of Fmoc-Pro-OH, 3 equiv. of DIC, 1 equiv. of OximaPure, in 4.5 mL of DMF. Reaction for 5 min at 90° C. in the microwaved-assisted Liberty Blue peptide synthesizer. (b) Fmoc deprotection with 20% N-methylpiperidine in DMF twice for 3 minutes at 90° C. in the microwaved-assisted Liberty Blue peptide synthesizer; (c) Peptides growth using previous conditions on Liberty Blue system; (d) TFA/TIS/water/phenol (94:2:2:2), 5 hours at room temperature.

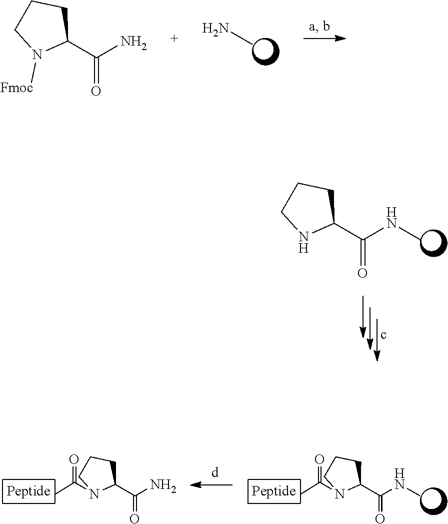

### Example 4. Synthetic Scheme for the Synthesis of Targefrin-Dimer

Conditions: (a) Rink Amide resin+3 equiv. of Fmoc-Lys(Fmoc)-OH, 3 equiv. of DIC, 1 equiv. of OximaPure, in 4.5 mL of DMF. Reaction for 5 minutes at 90° C. in the microwaved-assisted Liberty Blue peptide synthesizer; (b) Fmoc deprotection with 20% N-methylpiperidine in DMF twice for 3 minutes at 90° C. in the microwaved-assisted Liberty Blue peptide synthesizer; (c) Peptides growth using previous conditions but using double equivalents for dimer growth: 6 equiv. of Fmoc-Amino Acid, 6 equiv. of DIC, 2 equiv. of OximaPure, in 4.5 mL of DMF. Reaction for 5 minutes at 90° C. in the microwaved-assisted Liberty Blue peptide synthesizer; (d) TFA/TIS/water/phenol (94:2:2:2), 5 hours at room temperature.

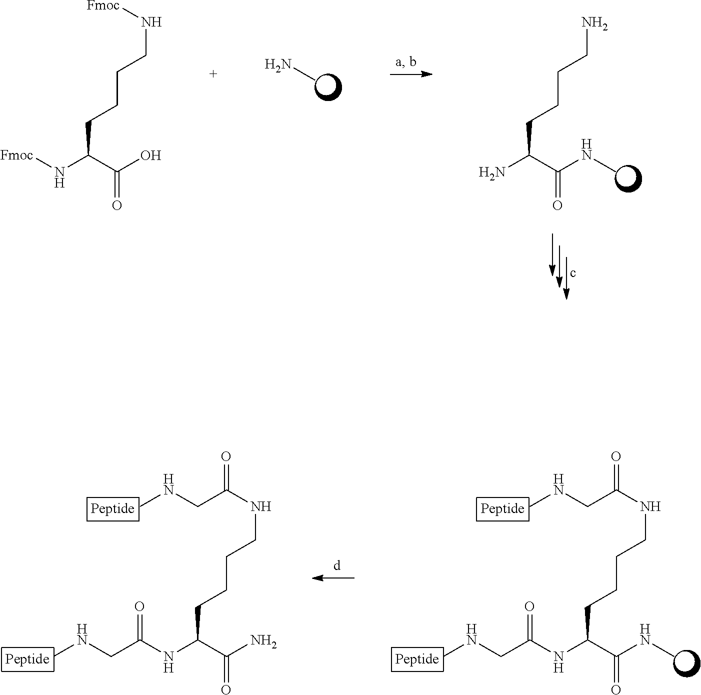

### Example 5. Synthetic Scheme for the Synthesis of Compound Targefrin-Motif (Intermediate to Synthesize Targefrin-Paclitaxel)

Conditions: (a) Rink Amide resin+3 equiv. of Fmoc-Lys(ivDde)-OH, 3 equiv. of HATU, 3 equiv. of OximaPure, and 5 equiv. of DIPEA in 1 mL of DMF, 1 hour at room temperature; (b) Fmoc deprotection with 20% piperidine in DMF twice; (c) 3 equiv. of Fmoc-Gly-OH, 3 equiv. of HATU, 3 equiv. of OximaPure, and 5 equiv. of DIPEA in 1 mL of DMF, 1 hour at room temperature; (d) Peptides growth using previous conditions or LibertyBlue; (e) ivDde deprotection using 4% N₂H₂ in DMF (3× 5 mL), room temperature; (f) 3 equiv. of 5-10 Hexynoic acid, 3 equiv. of HATU, 3 equiv. of OximaPure, and 5 equiv of DIPEA in 1 mL of DMF, 1 hour at room temperature; (g) TFA/TIS/water/phenol (94:2:2:2), 5 hours at room temperature.

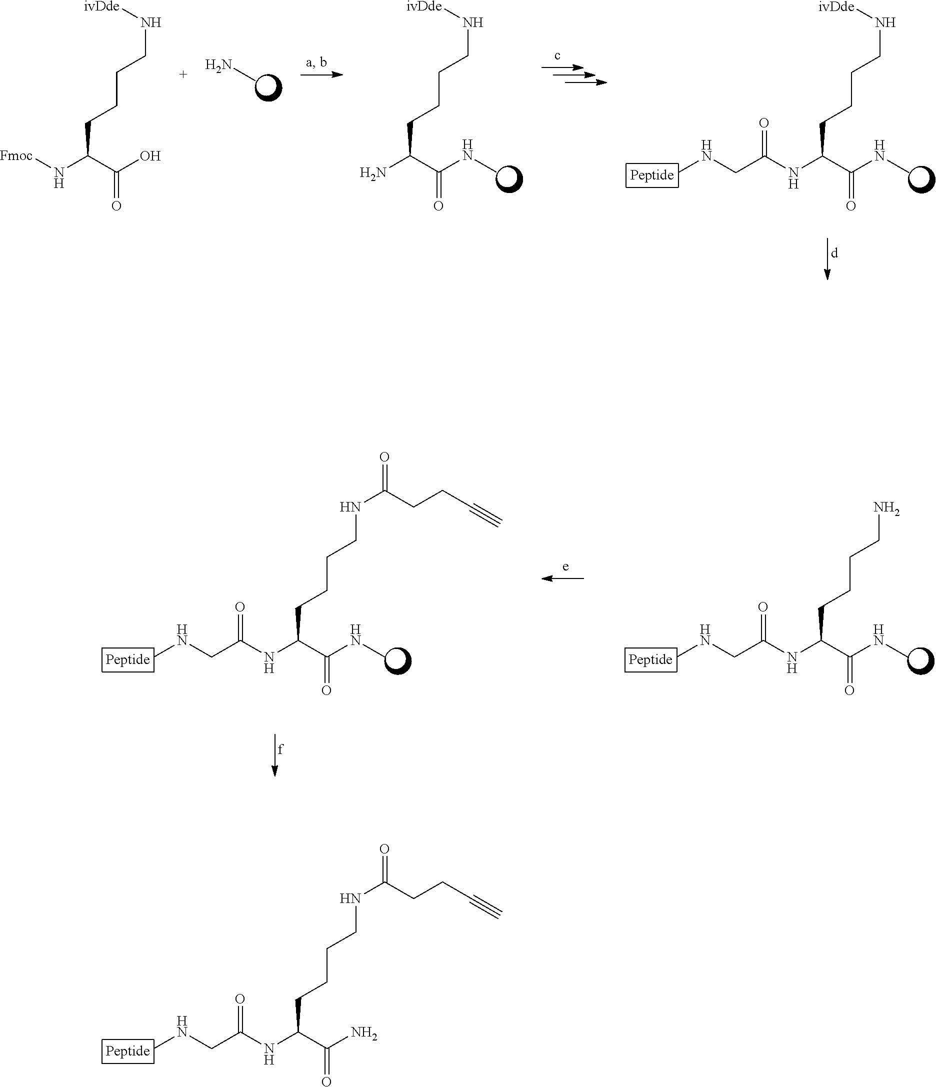

### Example 6. Synthetic Scheme for the Synthesis of Compound Targefrin-Dimer-Motif (Intermediate to Synthesize Targefrin-Dimer-Paclitaxel)

Conditions: (a) Rink Amide resin+3 equiv. of Fmoc-Lys(ivDde)-OH, 3 equiv. of HATU, 3 equiv. of OximaPure, and 5 equiv. of DIPEA in 1 mL of DMF, 1 hour at room temperature. (b) Fmoc deprotection with 20% N-methylpiperidine in DMF twice. (c) 3 equiv. of Fmoc-Gly-OH, 3 equiv. of HATU, 3 equiv. of OximaPure, and 5 equiv. of DIPEA in 1 mL of DMF, 1 hour at room temperature. (d) 3 equiv. of Fmoc-Lys(Fmoc)-OH, 3 equiv. of HATU, 3 equiv. of OximaPure, and 5 equiv. of DIPEA in 1 mL of DMF, 1 hour at room temperature. (e) Peptides growth double equivalents for dimer growth: 6 equiv. of Fmoc-Amino Acid, 6 equiv. of DIC, 2 equiv. of OximaPure, in 4.5 mL of DMF. Reaction for 5 minutes at 90° C. in the microwaved-assisted Liberty Blue peptide synthesizer. (f) ivDde deprotection using 4% N₂H₂ in DMF (3×5 mL), rt; (g) 3 equiv. of 5-Hexynoic acid, 3 equiv. of HATU, 3 equiv. of OximaPure, and 5 equiv of DIPEA in 1 mL of DMF, 1 hour at room temperature; (h) TFA/TIS/water/phenol (94:2:2:2), 5 hours at room temperature.

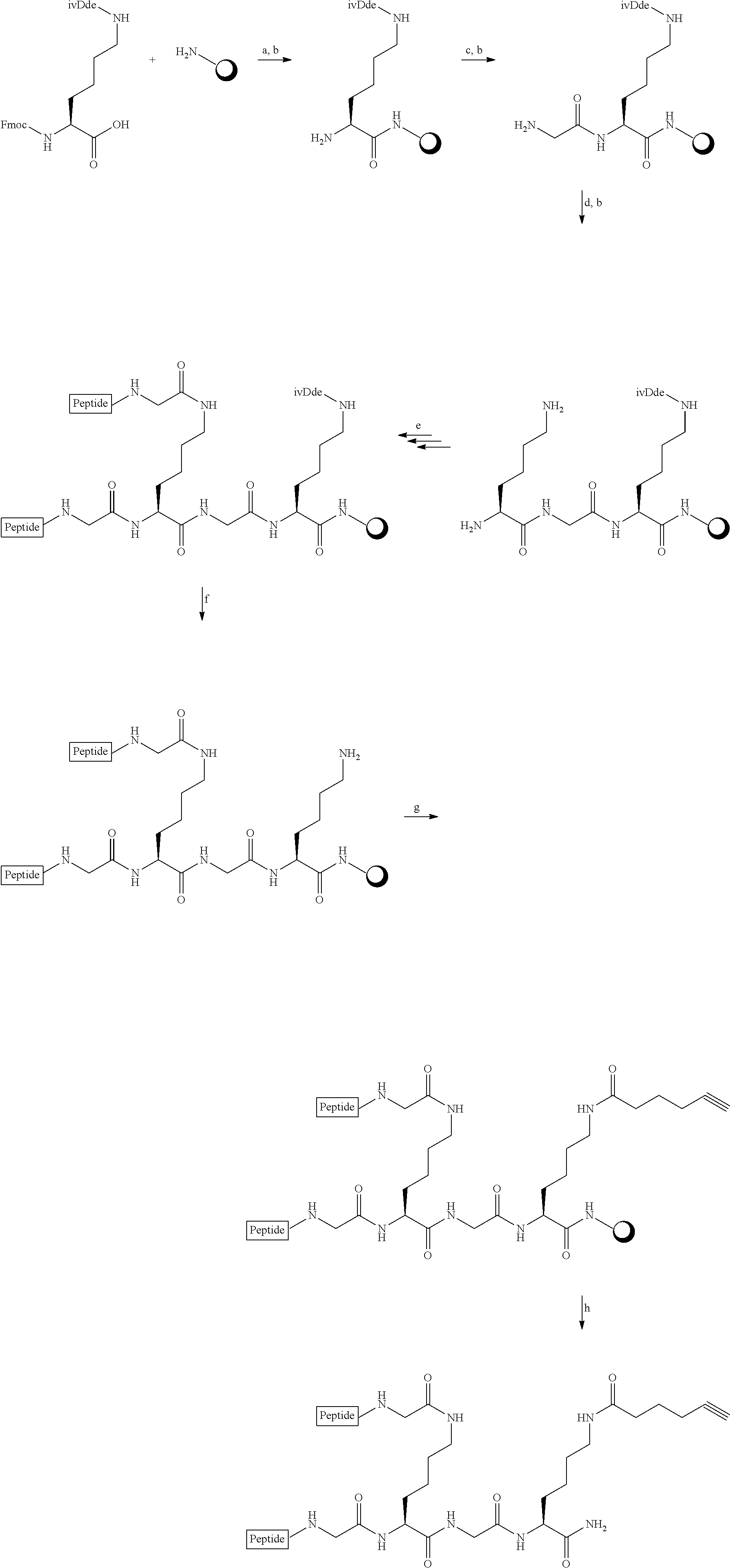

### Example 7. Synthetic Scheme for the Synthesis of Targefrin-PTX

Conditions: (a) Targefrin-motif crude, 1 equiv. of PTX-Azide in 4 mL of 4:1 DMSO:water solution. Add 50 μL of CuSO4 1M and 50 μL of Sodium Ascorbate 1M. Mix at room temperature for 48 hours.

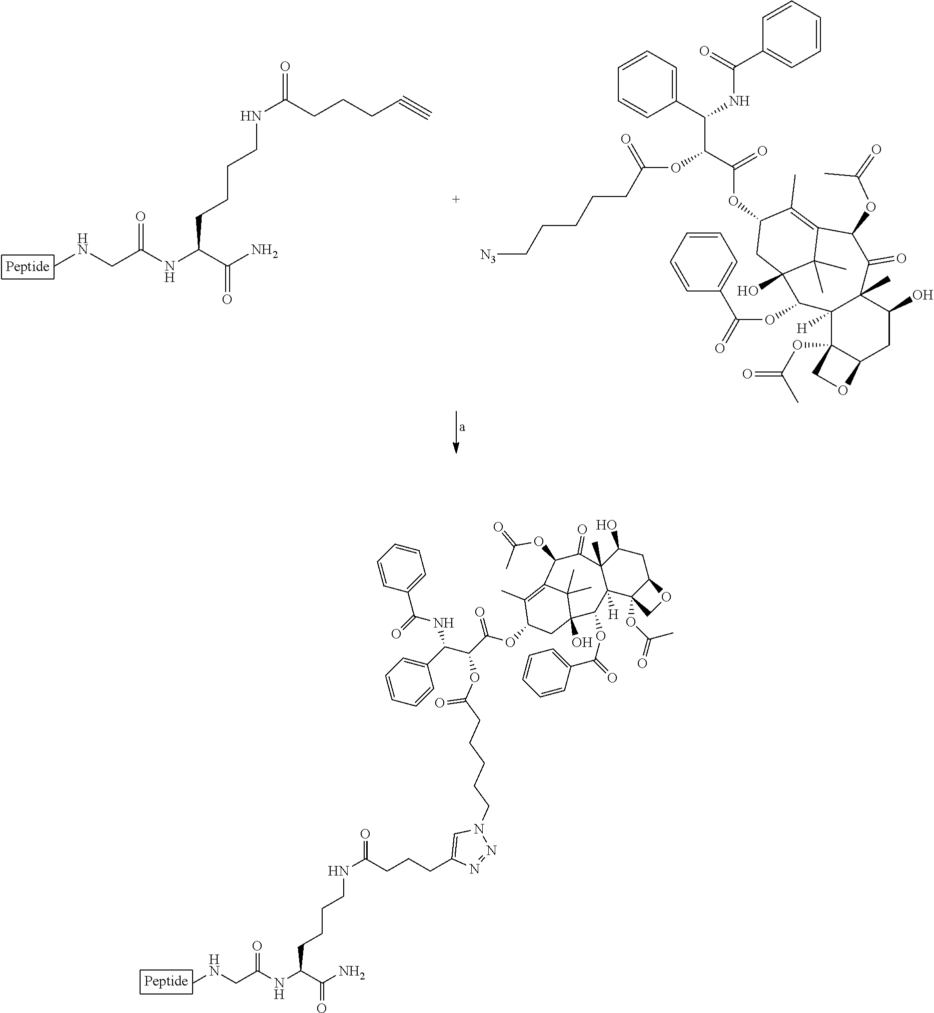 [SMILES: C#CCCCC(=O)NCCCC[C@H](NC(=O)CNC)C(N)=O ; CC(=O)O[C@H]1C(=O)[C@@]2(C)[C@H]([C@H](OC(=O)c3ccccc3)[C@]3(O)C[C@H](OC(=O)[C@H](OC(=O)CCCCCN=[N+]=[N-])[C@@H](NC(=O)c4ccccc4)c4ccccc4)C(C)=C1C3(C)C)[C@]1(OC(C)=O)CO[C@@H]1C[C@@H]2O ; CNCC(=O)N[C@@H](CCCCNC(=O)CCCc1cn(CCCCCC(=O)O[C@@H](C(=O)O[C@H]2C[C@@]3(O)[C@@H](OC(=O)c4ccccc4)[C@@H]4[C@]5(OC(C)=O)CO[C@@H]5C[C@H](O)[C@@]4(C)C(=O)[C@H](OC(C)=O)C(=C2C)C3(C)C)[C@@H](NC(=O)c2ccccc2)c2ccccc2)nn1)C(N)=O]

### Example 8. Synthetic Scheme for the Synthesis of Targefrin-Dimer-PTX

Conditions: (a) Targefrin-dimer-motif crude, 1 equiv. of PTX-Azide in 4 mL of 4:1 DMSO:water solution. Add 50 μL of CuSO₄ 1M and 50 μL of Sodium Ascorbate 1M. Mix at room temperature for 48 hours.

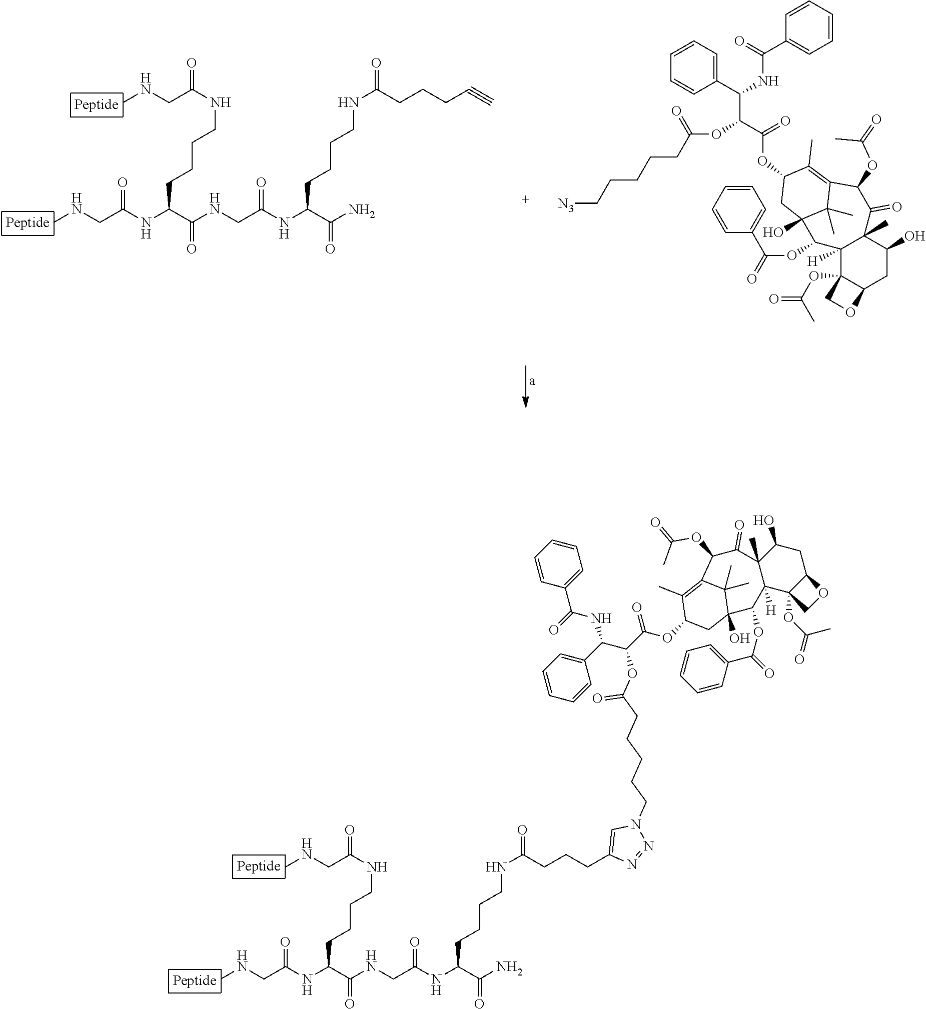

### Example 9. Targefrin Monomer Functions as an Antagonist

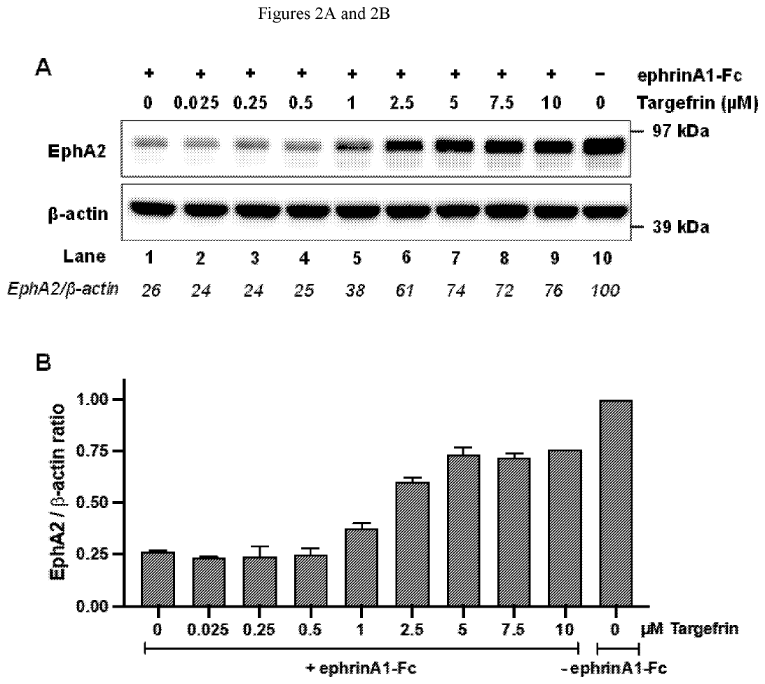

FIG. 2A shows Western blot of BxPC3 cells, which were starved for 1 hour and pre-treated with various concentrations of targefrin for 20 minutes followed by a combination treatment with 2 μg/mL ephrinA1-Fc for 3 hours. FIG. 2B shows quantification of EphA2 level. EphA2/β-actin ratios were normalized by designating the EphA2 expression from the DMSO without ephrinA1-Fc condition as 1. EC₅₀ value was calculated to be 1.6±0.1 μM and was presented as mean±standard error (SE) of 2 independent experiments.

### Example 10. Chemical Structures of the Dimeric EphA2 Binding Agents

IC₅₀ values were obtained by replicate DELFIA measurements against EphA2-LBD.

TABLE 3
Chemical structures of the dimeric EphA2 binding agents. IC₅₀ values were
obtained by replicate DELFIA measurements.

| | | IC₅₀ |
|---|---|---|
| ID | STRUCTURE | (nM) |
| 135H12 | 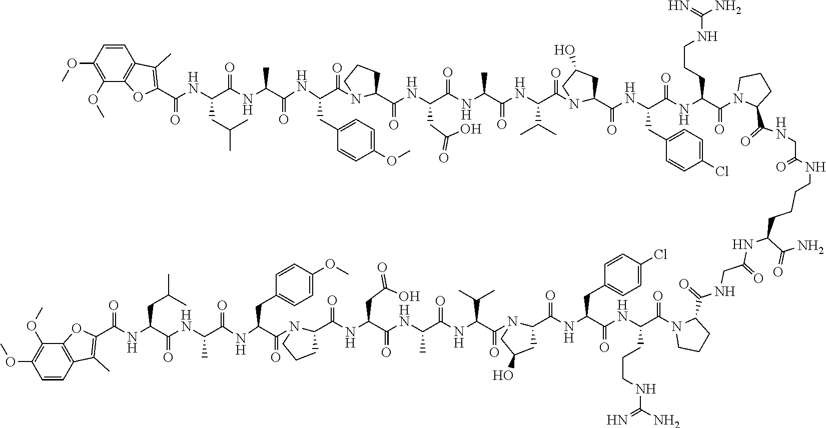 | 60 ± 12 |
| 28 (tar- gefrin- dimer) n = 1 | 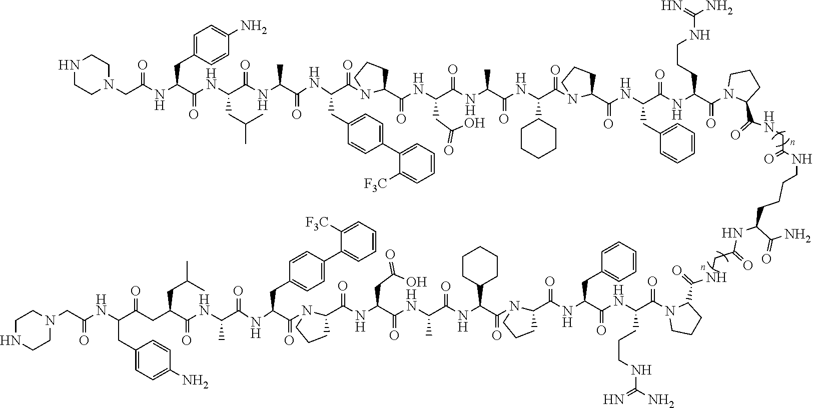 | 6.5 ± 0.6 |
| 29 | | 7.6 ± |
| n = 2 | | 0.7 |
| 30 | | 5.3 ± |
| n = 3 | | 0.6 |

### Example 11. Targefrin-Dimer and its Variations Cause EphA2 Degradation at Nanomolar Concentrations in Pancreatic Cancer Cell Lines

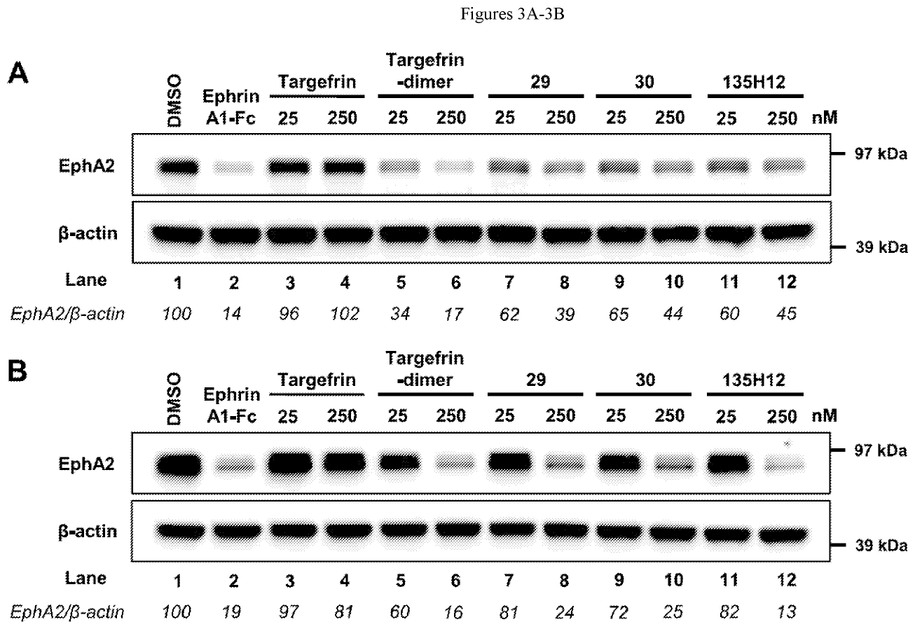

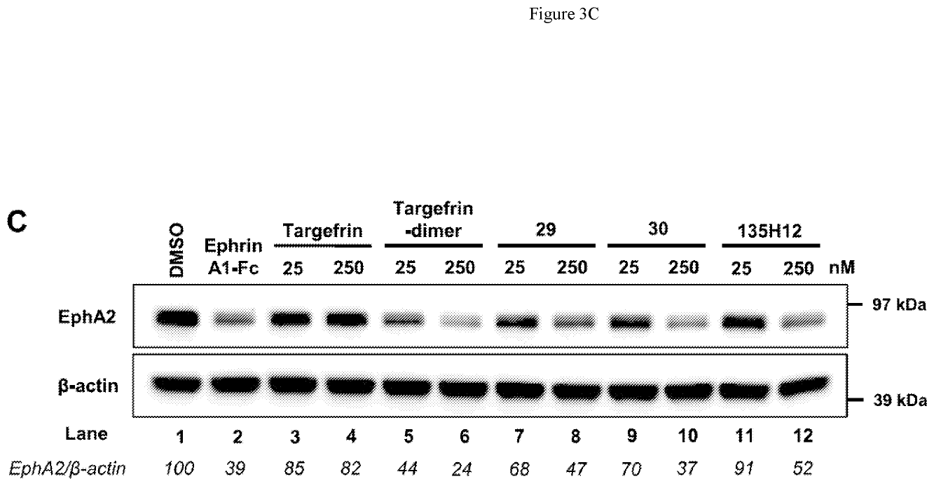

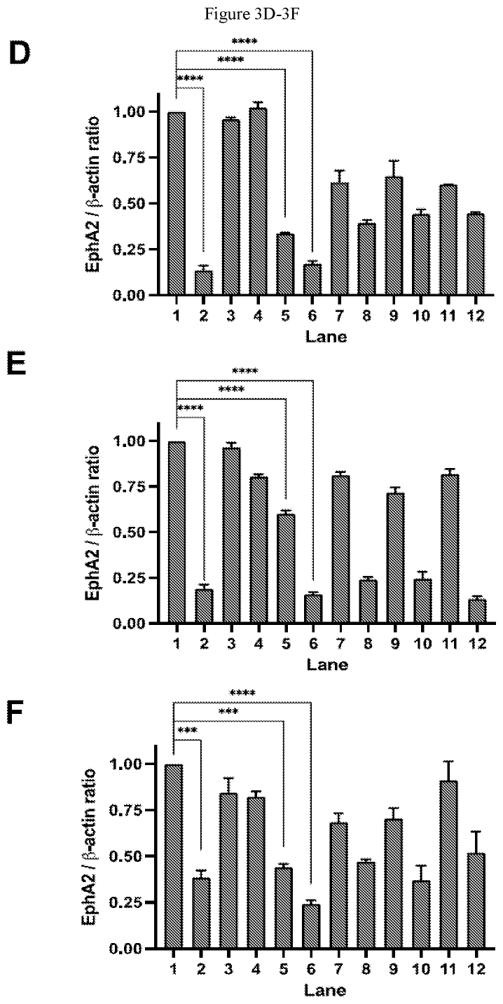

FIGS. 3A-3C show western blot images of BxPC3, PANC-1 and MIA PaCa-2 cells, respectively, in which cells were starved for 1 hour and treated with 2 μg/mL ephrinA1-Fc or the indicated doses of targefrin, targefrin-dimer and its variations with different linkers for 3 hours. Dimeric agent 135H12 (see US20210221843A1; PCT WO2019237075A1; and Gambini et al. ACS Chem Biol. 2018, 13 (9), 2633-2644). is shown as reference. FIGS. 3D-3F show densitometry analyses for the data shown in FIGS. 3A-3C, respectively. EphA2/β-actin ratios were normalized by designating the EphA2 expression from the DMSO control condition as 100% for FIGS. 3A-3C or 1 for FIGS. 3D-3F. ***p<0.001. ****p<0.0001, as determined by a one-way analysis of variance using Dunnett's post-test analysis.

### Example 12. Chemical Structure and Biochemical Activity of Targefrin-Conjugated Agents

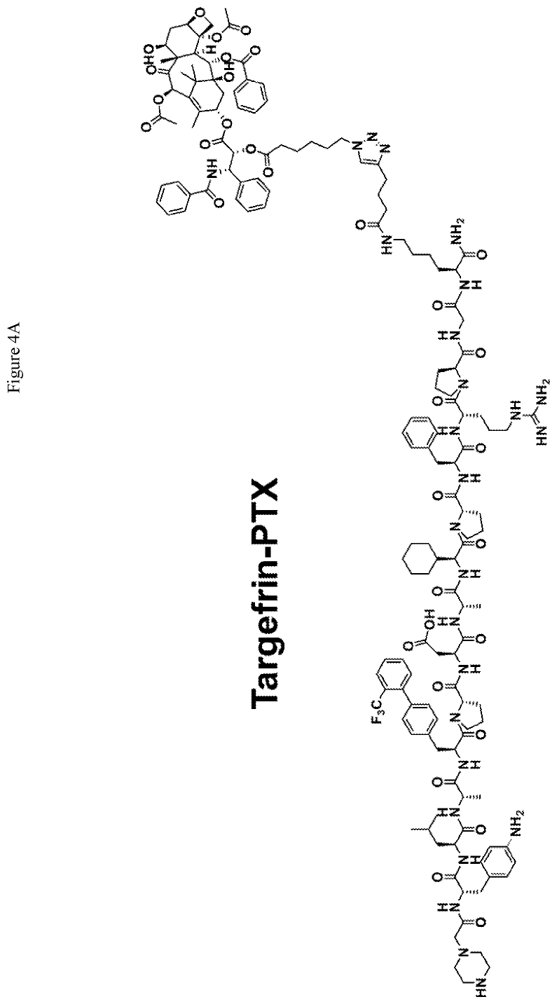

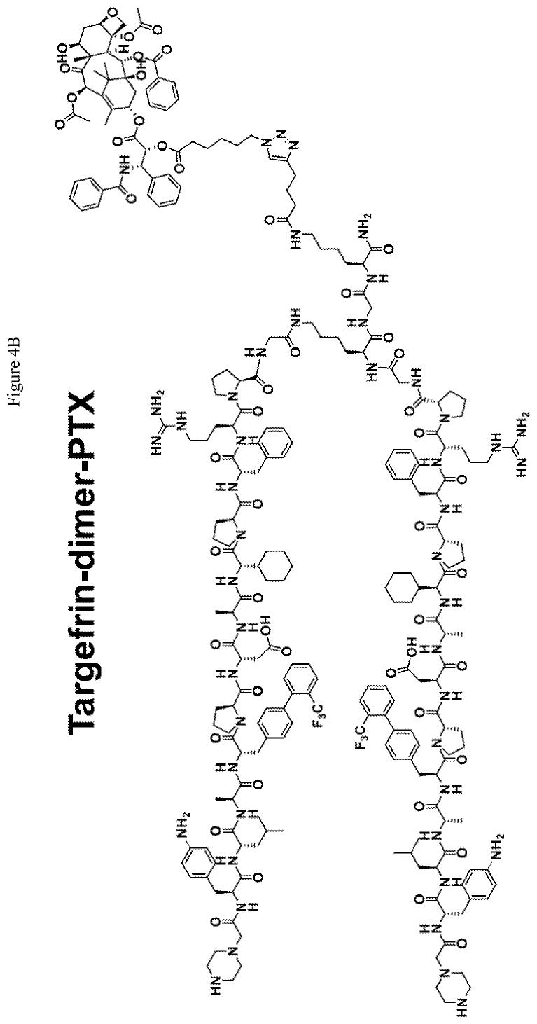

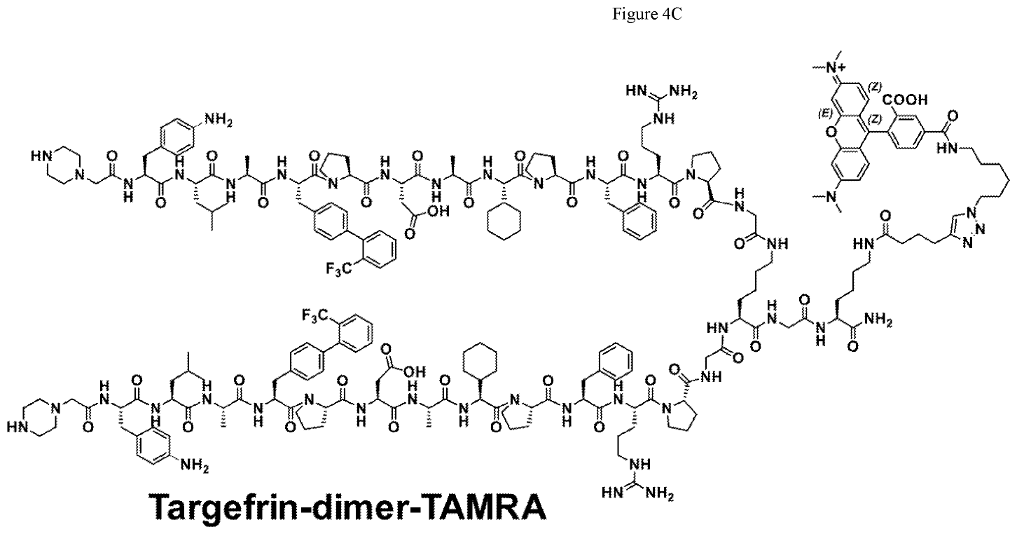

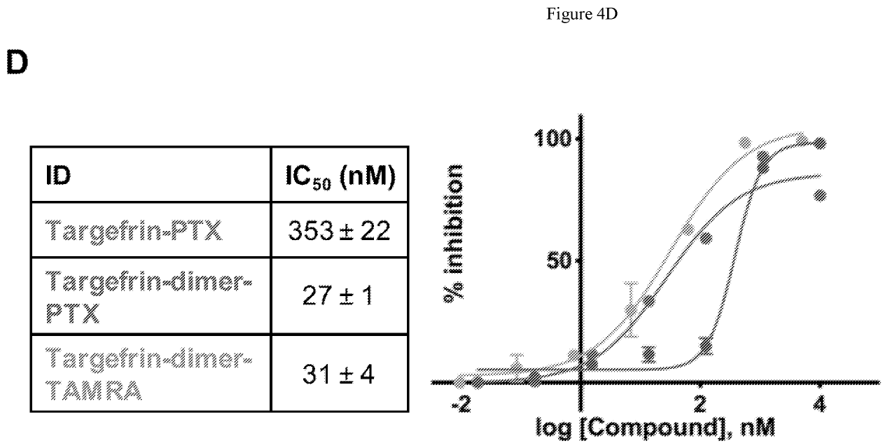

FIG. 4A shows the chemical structures of Targefrin conjugated to paclitaxel (targefrin-PTX). FIG. 4B shows the dimeric version of targefrin conjugated to paclitaxel (targefrin-dimer-PTX). FIG. 4C shows the dimeric version of targefrin conjugated to the 5-carboxytetramethylrhodamine-azie dye (targefrin-dimer-TAMRA). FIG. 4D shows the DELFIA displacement dose-response curves comparing targefrin-PTX, targefrin-dimer-PTX, and targefrin-dimer-TAMRA, with their respective IC₅₀ values.

### Example 13. Targefrin-Dimer-TAMRA is Internalized in EphA2-Expressing Cells

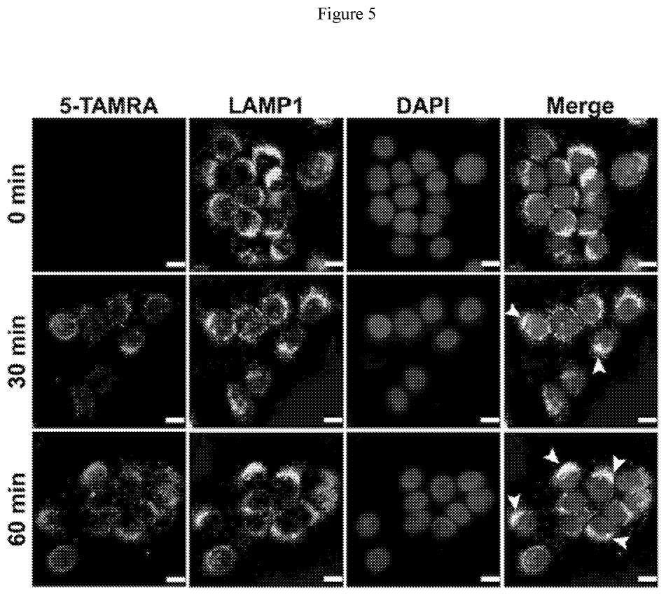

BxPC3 cells were treated with 100 nM targefrin-dimer-TAMRA for 0, 30 and 60 minutes. Upon binding of the agent, EphA2 was internalized and targeted to the lysosomes as shown by colocalizations of 5-TAMRA and LAMP1 (arrowheads). See FIG. 5. Scale bar=10 μm.

### Example 14. Targefrin-Dimer Significantly Inhibits Pancreatic Cancer Cell Migration

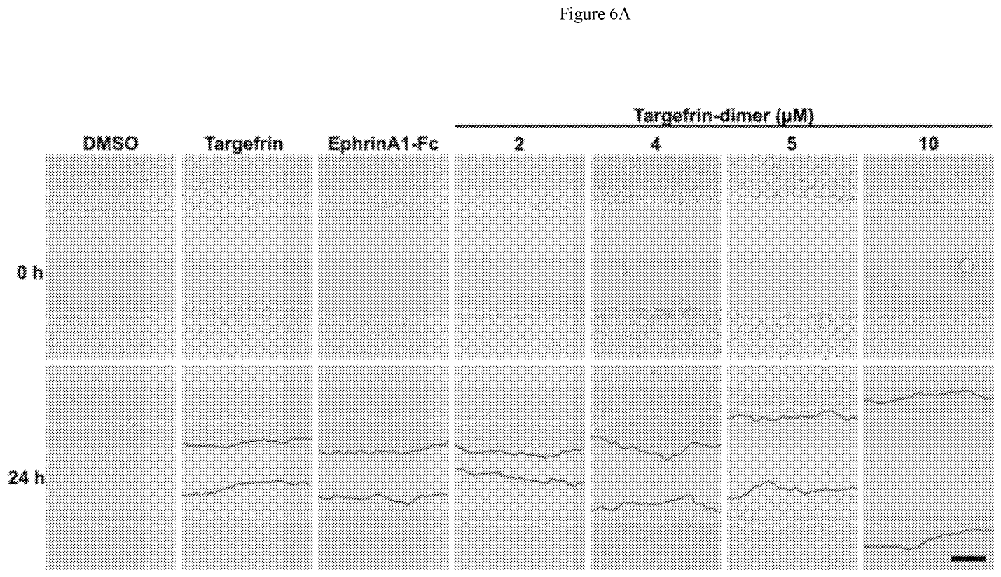

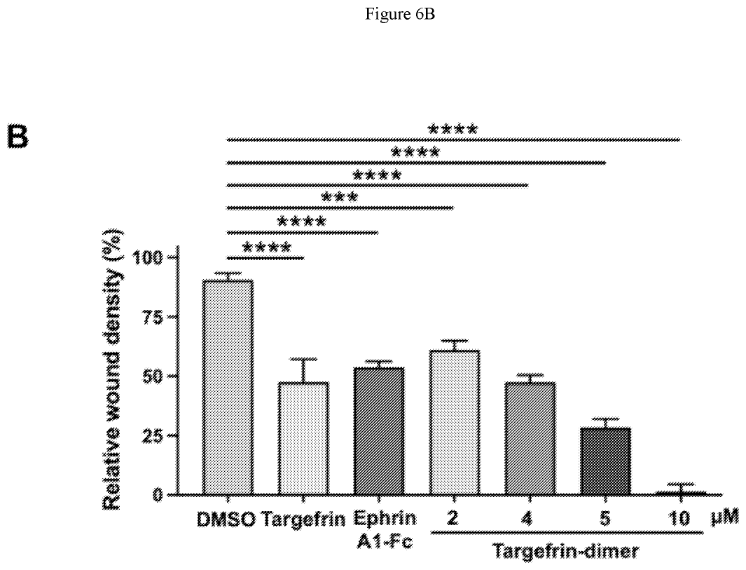

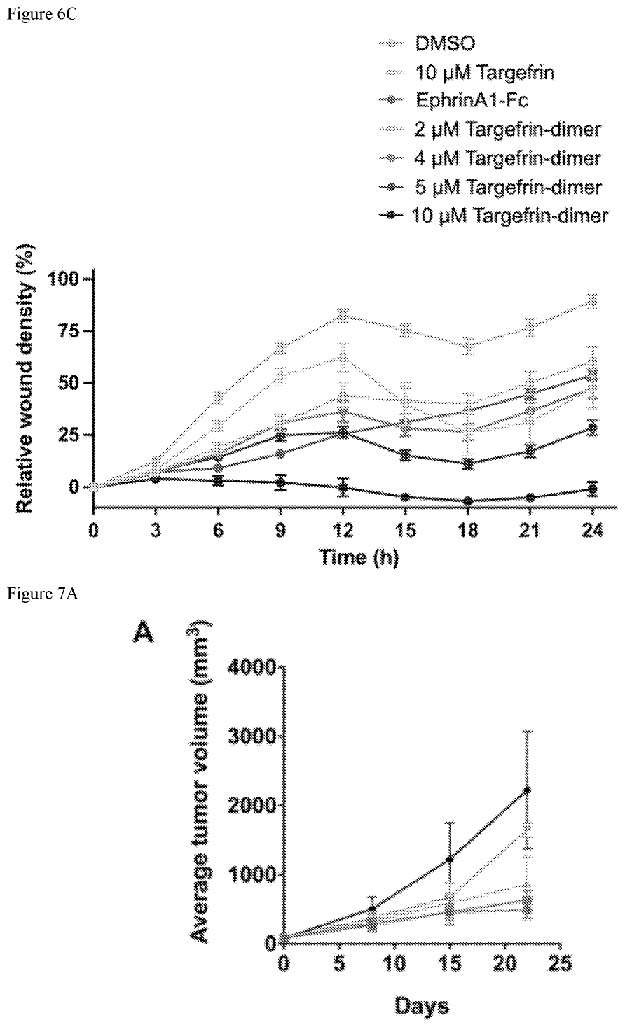

FIG. 6A shows Cell migration assay of BxPC3 treated with 2 μg/mL ephrinA1-Fc and 10 μM targefrin or the indicated doses of targefrin-dimer. Plates were imaged every 3 hours for 24 hours. FIG. 6B shows that the Targefrin-dimer significantly inhibited cell migration at 24 hours in a dose-dependent manner as shown by decreases in relative wound density. FIG. 6C shows how time-response curves showed the effects of the agents on wound closure over a period of 24 hours. ***p 0.001, ****p<0.0001, as determined by a one-way analysis of variance using Dunnett's post-test analysis. Scale bar=250 μm.

### Example 15. PTX-Conjugated Suppress Tumor Growth in a Tumor Xenograft with MIA-PaCa-2 Cells

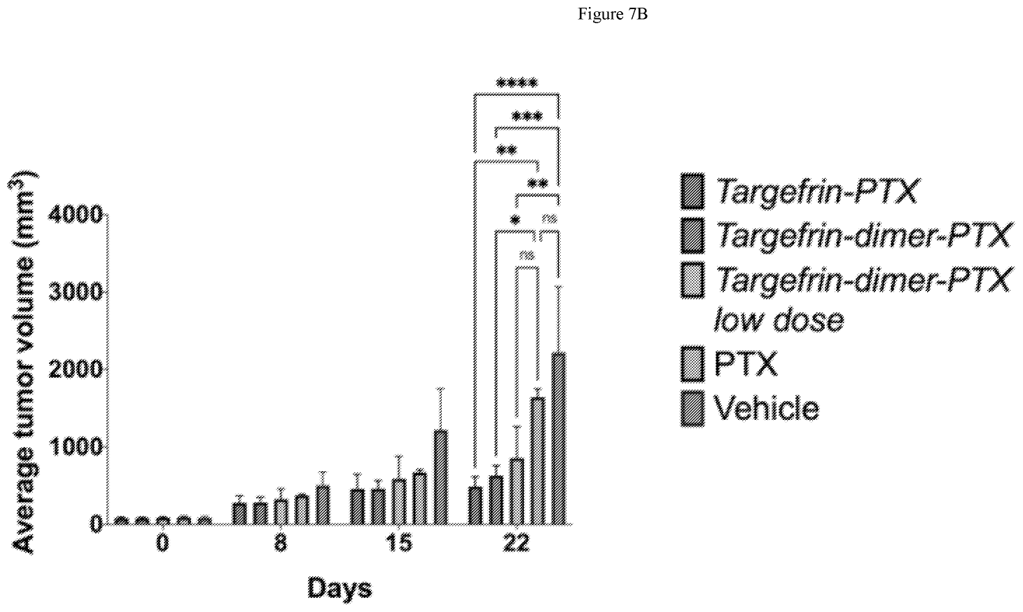

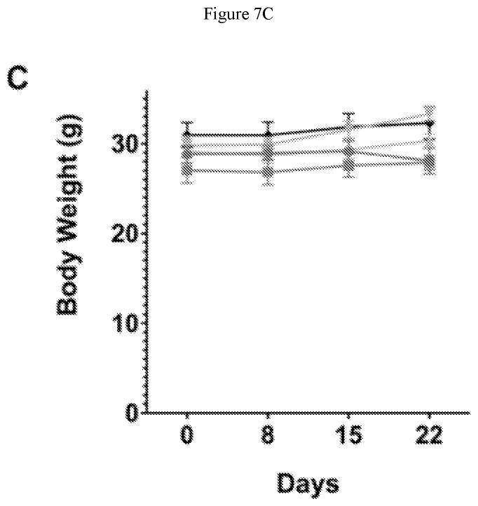

FIG. 7A shows five groups of 5 mice bearing preestablished MIA-PaCa-2 tumors, were treated for 22 days with vehicle control alone, paclitaxel (PTX; 2.5 mg/Kg), targefrin-PTX (10 mg/Kg which is equivalent to 2.5 mg/Kg of PTX), targefrin-dimer-PTX (17 mg/kg which is equivalent to 2.5 mg/Kg of PTX) and a lower dose of targefrin-dimer-PTX (10 mg/kg which is equivalent to 1.5 mg/kg of PTX). The tumor volume is reported as average±SE. FIG. 7B shows the average tumor volume for each group of treatment, measured at 0, 8, 15, and 22 days. *p=0.03, **p<0.01, ***p=0.0001, ****p<0.0001, as determined by a two-way analysis of variance using Tukey's post-test analysis. FIG. 7C shows the average body weight±SE is reported for each of the five group of treatment at 0, 8, 15, and 22 days.

### Example 16. Representative Pharmaceutical Dosage Form

The following illustrates a representative pharmaceutical dosage form containing a compound of formula I (‘Compound X’), for therapeutic or prophylactic use in humans. IV injectable formulation consisting of 80% PBS, 10% Tween 80, 10% Ethanol with agent dissolved at up to 20 mg/ml. This and several variations on this formulation may be obtained by conventional procedures well known in the pharmaceutical art.

### Example 17. Synthetic Scheme for the Synthesis of Targefrin-Dimer-TAMRA

Conditions: (a) Targefrin-dimer-motif crude, 1 equiv. of 5-TAMRA-Azide in 4 mL of 4:1 DMSO:water solution. Add 50 μL of CuSO4 1M and 50 μL of Sodium Ascorbate 1M. Mix at rt for 48 h.

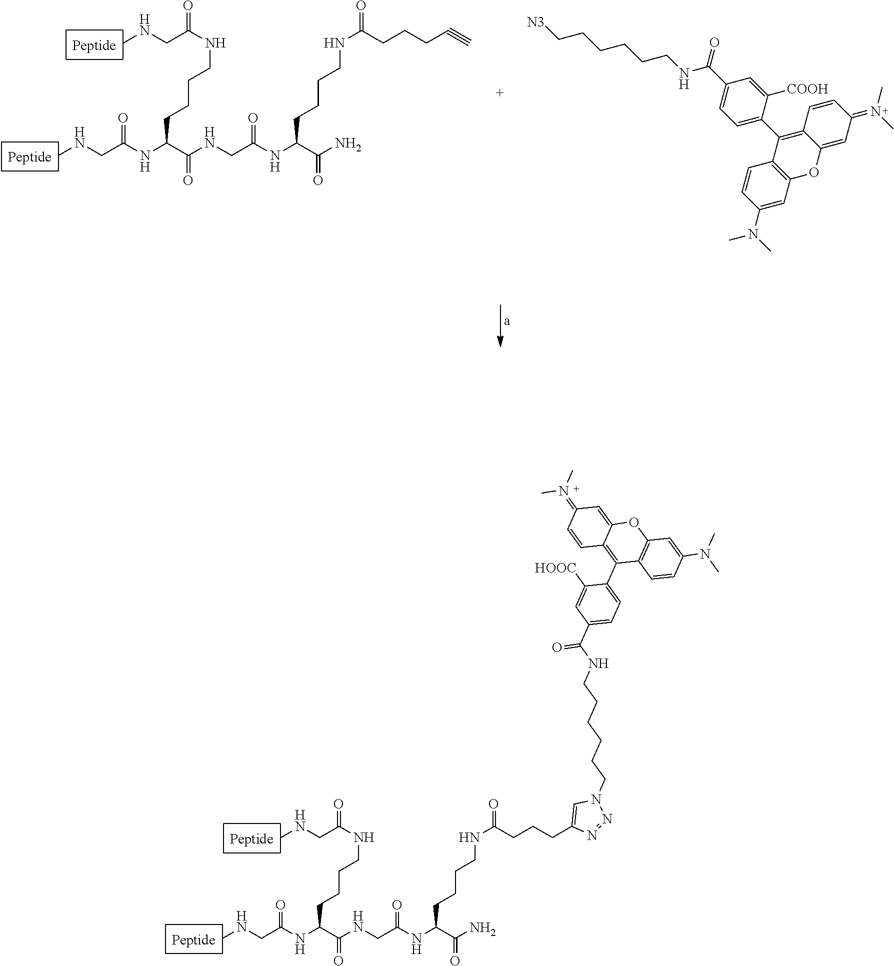

### Example 18. Targefrin: A Potent Agent Targeting the Ligand Binding Domain of EphA2

Overexpression of the receptor tyrosine kinase EphA2 is associated with poor prognosis and development of aggressive metastatic cancers. Guided by our recently solved X-ray structure of the complex between an agonistic peptide and EphA2-LBD, this Example describes a novel agent, targefrin, that binds to EphA2-LBD with a 21 nM dissociation constant by isothermal titration calorimetry and presents an ICs value of 10.8 nM in a biochemical assay. In cell-based assays, a dimeric version of the agent is as effective as the natural dimeric ligands (ephrinA1-Fc) in inducing cellular receptor internalization and degradation in several pancreatic cancer cell lines. When conjugated with chemotherapy, the agents can effectively deliver paclitaxel to pancreatic cancers in a mouse xenograft study. Given the pivotal role of EphA2 in tumor progression, the agents reported herein could be further developed into innovative EphA2-targeting therapeutics.

### INTRODUCTION

The receptor tyrosine kinase EphA2 in its ephrin-bound-form functions as a tumor suppressor, preventing cancer cell migration, tumor growth, and angiogenesis. On the contrary, when the receptor is in its unbound state, such as when it is aberrantly overexpressed, it confers cancers cells pro-oncogenic traits inducing metastatic behavior in several solid tumors including pancreatic cancer,²⁻⁴ prostate cancer,⁵⁻⁷ breast cancer,⁸⁻¹⁰ esophageal cancer,^{11 12} melanoma,¹³ urinary bladder,¹⁴ brain cancer,¹⁵⁻¹⁷ lung cancer,¹⁸ ovarian cancer,¹⁹ stomach cancer,²⁰ and some types of leukemia.²¹⁻²⁴ Hence, due to its role as tumor suppressor, targeting EphA2 is being targeted for the development of various possible therapeutic strategies, including targeting its intracellular kinase domain,²⁵⁻²⁸ or its ligand binding domain.^{29,30} While the unbound EphA2 receptor functions as potent oncogene, its tumorigenic effect could be suppressed, and perhaps reverted, by synthetic agents that mimic its ligand, the membrane-anchored ephrinA1.³¹

In cellular assays, when a chimeric protein consisting of ephrinA1 and the Fc region of an antibody, ephrinA1-Fc, engages with the EphA2 ligand binding domain (LBD), it causes receptor dimerization, followed by clustering and internalization that results in the degradation of the receptor via a lysosomal pathway.³² Therefore, because ephrinA1-Fc could in principle revert pro-oncogenic EphA2 into a tumor suppressor, the design of potent and effective ephrinA1-Fc mimetics holds potential for the development of novel anti-metastatic therapeutics. Because such agents may cause receptor internalization, these could be additionally deployed as carrying molecules for selective targeted delivery of chemotherapy to EphA2 expressing cancers. In this regard, we have recently developed an EphA2 dimeric agonistic peptide mimetic that, similar to ephrinA1-Fc, could suppress tumor metastases in an orthotropic model of prostate cancer,³³ and suppresses cell migration in pancreatic cancer cell lines.³⁴ When certain earlier agents were conjugated with the chemotherapeutic agents gemcitabine³⁵ or paclitaxel^{8, 36-37} these resulted in delivery of their cargo to EphA2 expressing tumors, including pancreatic cancer,³⁵ prostate cancer,³⁶⁻³⁷ breast cancer,^{8, 38}, and melanoma.³⁸ More recently, we solved the X-ray structure for the first time of an agonistic ephrin peptide mimetic in complex with EphA2-LBD.¹ Leveraging on previous structure-activity relationship studies on previous peptide binders from our laboratory^{1, 37-38} and the high-resolution X-ray structure,¹ we sought here to further derive agents that could approach the affinity and activity of ephrinA1-Fc in targeting EphA2-LBD. As shown herein, a novel agent with low nanomolar affinity for EphA2-LBD was identified, which presents a comparable affinity for the receptor as ephrinA1. In cellular assays, a dimeric version of our most potent agent (we termed targefrin) induces receptor degradation at nanomolar concentrations, similar to the effect of ephrinA1-Fc, as assessed by western blot analysis in pancreatic cancer cell lines BxPC3, PANC-1, and MIA PaCa2, representing KRAS wild-type (BxPC3) and KRAS-mutant (PANC-1 and MIA PaCa2) tumors. In phenotypic assays, the agents are also effective in suppressing cell migration in the BxPC3 pancreatic cancer cell line. When conjugated with paclitaxel, the agent is effective in suppressing tumor growth in a MIA PaCa2 xenograft model of pancreatic cancer. The extraordinary affinity of targefrin for the ligand binding domain of EphA2 makes this agent an unprecedented pharmacological tool to study this receptor tyrosine kinase and for the development of novel therapeutics and/or targeted delivery strategies.

### Results

### Design, Synthesis, and Characterization of Targefrin

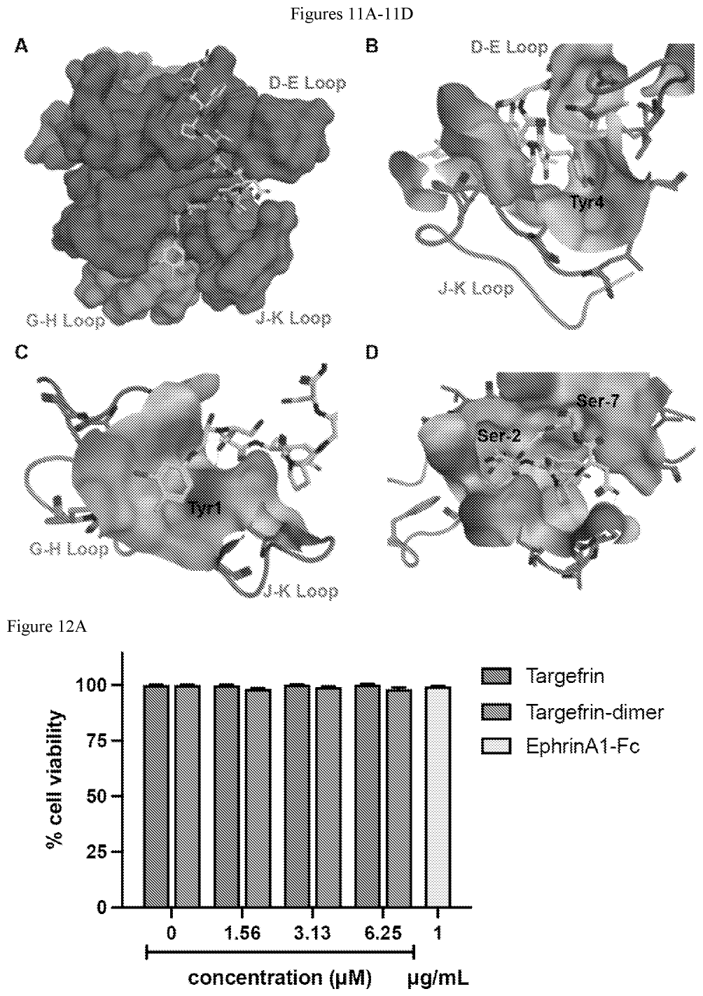

In order to rapidly and iteratively characterize the binding properties of novel EphA2 binding ligands (Table 4), we performed isothermal titration calorimetry binding measurements, using recombinant EphA2 ligand binding domain (LBD). An earlier agent in complex with EphA2-LBD (PDB ID 6B9L) is shown in FIG. 11A.¹ Using ligand of sequence YSAYPDSVPFRP (K_{d} 1230 nM, ITC; Table 4, compound 1), that merged the sequences of the phage display derived YSA peptide 39 with the sequences of natural ephrin ligands,¹ we started exploring optimization strategies (FIG. 11). First, we probed substitutions that could protrude into a large hydrophobic pocket located in proximity of the Tyr 4 of the peptide (FIGS. 11A-11B, Table 4). Here, replacement of the Tyr residue in position 4 with bulkier aromatic groups enhanced the affinity significantly (Table 4). Hence, subsequently, fixing a phenyl-Phe in position 4 of the peptide, we explored modifications at other positions.

TABLE 4
Structure-Activity Relationship studies in position 4 for the reported EphA2
binding agents. Chemical structures and dissociation constants are reported. K_{d} values were
obtained by reverse isothermal titration calorimetry measurements.

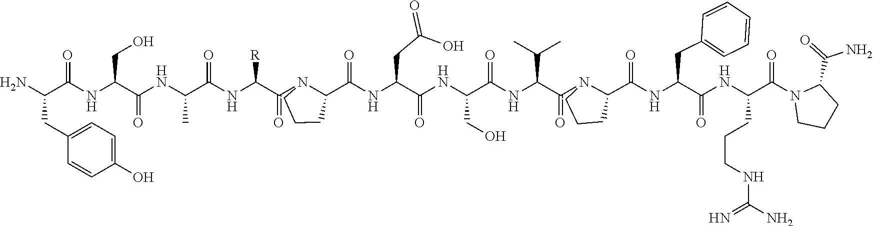

| | | K_{d} (nM)- |
|---|---|---|
| ID | R | ITC |
| 1 | 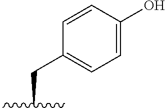 | 1230 |
| 2 | 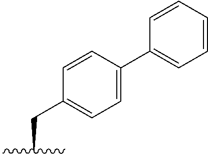 | 237.5 ± 4.0 (n = 4) |
| 3 | 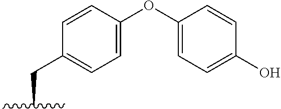 | 107 |
| 4 | 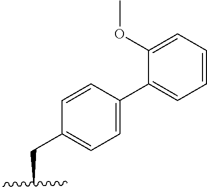 | 93 |
| 5 | 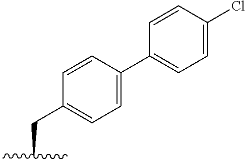 | 158 |
| 6 | 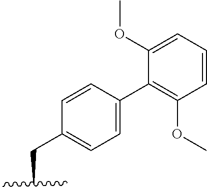 | 323 |
| 7 | 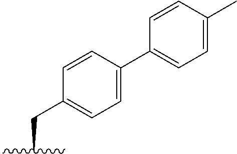 | 152 |
| 8 | 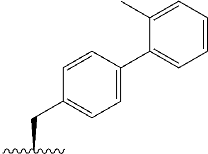 | 65 |
| 9 | 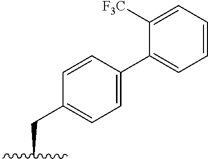 | 21 |
| 10 | 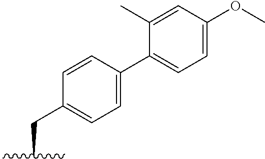 | 44 |

These included modifications of the N-terminal amide (FIGS. 11A,11C; Table 5), this position might not only be susceptible to amino-peptidases in plasma, but it is also involved in ligand recognition.^{1, 37-38} Replacement of the amino group with a piperazine or a morpholino increased the binding affinity for EphA2-LBD (Table 5). Moreover, we explored additional modifications, including modification of the Tyr residue in position 1, and the pair of Ser residues (Table 5). We found that Tyr 1 could be replaced by a variety of substituents thus eliminating the potential pharmacological liability represented by the phenolic hydroxyl group (Table 5). In its bound state, the peptide assumes a closed conformation with the two Ser residues forming intramolecular hydrogen bonding in the initial peptide (FIGS. 11A,11D). We further explored additional modifications of this pair of amino acids and assessed their influence on the binding affinity for EphA2-LBD via ITC measurements (Table 5).

Finally, a set of peptides were synthetized that contained optimal substituents from the agents reported in Tables 4 and 5, resulting in the final agents listed in Table 2. For these compounds we also assessed their binding properties using an orthogonal biochemical displacement assay based on the DELFIA platform, as described previously.¹

TABLE 5
Chemical structures and dissociation constants for EphA2 binding agents. K_{d}
values were obtained by reverse isothermal titration calorimetry measurements.

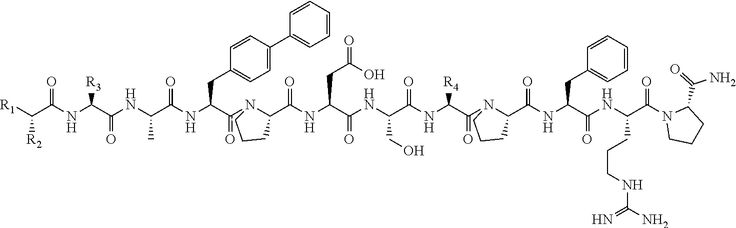

| | | | | | K_{d} (nM)- |
|---|---|---|---|---|---|
| ID | R₁ | R₂ | R₃ | R₄ | ITC |
| 2 | NH₂ | 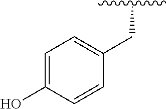 | 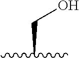 | 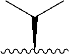 | 237.5 ± 4.0 (n = 4) |
| 11 | 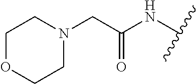 [SMILES: *NC(=O)CN1CCOCC1] | 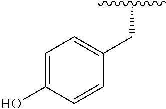 | 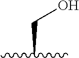 | 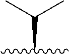 | 115 |
| 12 | 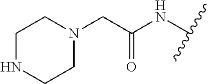 [SMILES: *NC(=O)CN1CCNCC1] | 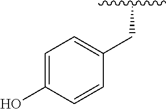 | 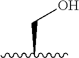 | 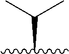 | 87 |
| 13 | NH₂ | 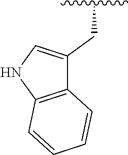 | 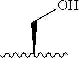 | 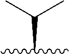 | 275 |
| 14 | NH₂ | 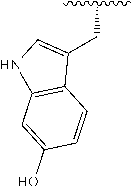 | 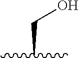 | 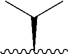 | 187 |
| 15 | NH₂ | 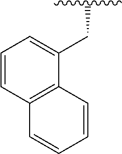 | 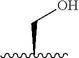 | 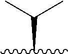 | 202 |
| 16 | NH₂ | 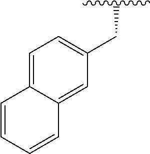 | 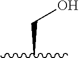 | 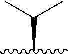 | 146 |
| 17 | NH₂ | 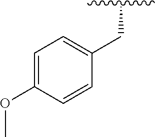 | 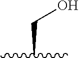 | 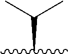 | 173 |
| 18 | NH₂ | 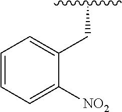 | 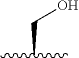 | 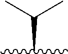 | 204 |
| 19 | NH₂ | 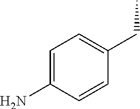 | 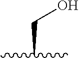 | 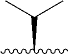 | 190 |
| 20 | NH₂ | 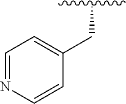 | 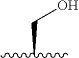 | 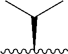 | 376 |
| 21 | NH₂ | 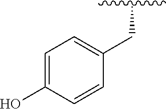 | 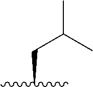 | 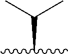 | 78 |
| 22 | NH₂ | 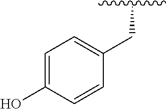 | 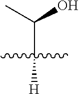 | 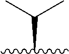 | 320 |
| 23 | NH₂ | 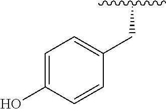 | 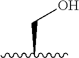 | 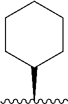 | 87 |

These studies culminated in the selection of agent 27, we term here targefrin, with an IC₅₀ value of 10.8 nM for EphA2-LBD (Table 2). FIG. 1 reports a molecular model of targefrin in complex with EphA2-LBD based on the X-ray structure of the complex with one of one our earlier peptide mimetic (PDB ID 6B9L).¹ To obtain a preliminary yet significant snapshot on the selectivity of targefrin for EphA2-LBD, compared to other members of this protein family, we tested it against the ligand binding domains of EphA3 and EphA4 that are the two Eph receptors with the greatest similarities to EphA2 (58% identity with EphA3-LBD, and 57% identity with EphA4-LBD). The ligand resulted inactive against both domains when tested under similar experimental conditions (FIG. 1D).

Monomeric peptides may elicit agonistic activities only at very high concentrations and act practically as antagonists at physiologically attainable concentrations.^{8, 34, 40} In agreement with its high affinity for EphA2, pre-treatment of the BxPC3 pancreatic cancer cells with targefrin effectively antagonized EphA2 degradation induced by the potent ephrinA1-Fc ligand, with an approximate EC₅₀˜1.6 μM under these experimental conditions (FIG. 2).

The agent alone did not induce appreciable EphA2 degradation in BxPC3 pancreatic cancer cells (FIG. 3).

However, while the monomeric peptides act as antagonists, similar to ephrinA1, dimerization of EphA2 targeting agents may result in compounds with increased agonistic activity in cell.^{1, 8, 33-34, 41-42} This is due presumably to the fact that enhancing dimerization may facilitate subsequent receptor clustering and internalization.^{8, 42} We prepared dimeric versions of targefrin (Table 3), using a Lys residue as a dimerization linker, spaced by Gly, β-Ala, or γ-amino butyric acid at the C-terminus of targefrin (Table 3). Table 3 also reports our previously identified dimeric agent 135H12.¹

### Targefrin-Dimer and Targefrin-Drug Conjugates

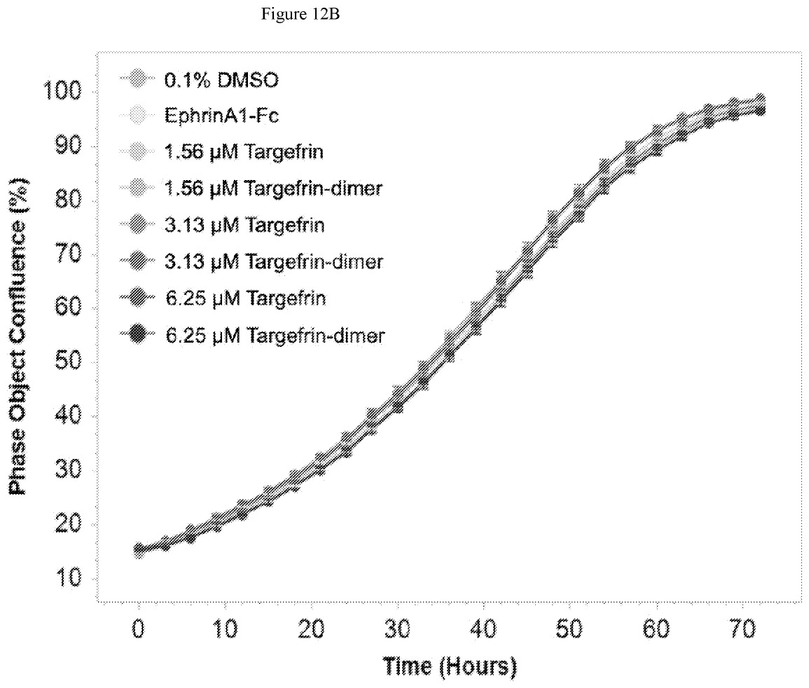

A property of agonistic agents that is of interest is that they induce EphA2 receptor internalization via a lysosomal pathway that causes its degradation. Hence, potent agonistic agents could induce EphA2 degradation, therefore eliminating its pro-oncogenic effects. The EphA2 internalization induced by agonistic agents would not necessarily affect cell proliferation, as in FIG. 12. However, due to the lysosomal internalization event, EphA2 agonistic agents could be used for targeted delivery of cytotoxic chemotherapy by synthesizing suitable peptide-drug conjugates (PDCs). Hence, to assess the EphA2 internalization and degradation properties of our agents we tested them in a variety of pancreatic cancer cell lines, side by side with dimerized ephrinA1-Fc as positive control. As reported above, when tested at nanomolar concentrations, the monomeric version of targefrin is not active in causing EphA2 degradation, in agreement with our previous observations that monomeric peptides are agonistic only at higher micromolar concentrations. This appears to be the case for all 3 cell lines tested, BxPC3, PANC-1, and MIA PaCa2 (FIG. 3).

However, dimeric versions of targefrin displayed a markedly increased receptor activation especially with the dimer having the Gly-Lys linker (Table 3) causing receptor degradation at sub-micromolar concentrations for all the pancreatic cancer cell lines tested (FIG. 3). Moreover, our newer agents are markedly more effective than our previously reported dimeric agent 135H12 (Table 3, FIG. 3).¹

To assess the utility of targefrin and targefrin-dimer as carriers for targeted delivery, we synthesized and tested drug conjugates including the chemotherapeutic agent paclitaxel and the fluorescent dye TAMRA (FIG. 4). The synthesis of these agents followed our previously described “click chemistry” linker that allows for an efficient incorporation in dimeric or monomeric agents of drugs or imaging reagents (Examples 7, 8, 17).^{1, 8} Conjugation of the dimeric-agents with TAMRA or paclitaxel did not significantly alter their binding properties for isolated EphA2-LBD (FIG. 4D), while a more significant loss in binding affinity was observed with the targefrin-monomer-PTX, perhaps due to the short linker chosen.

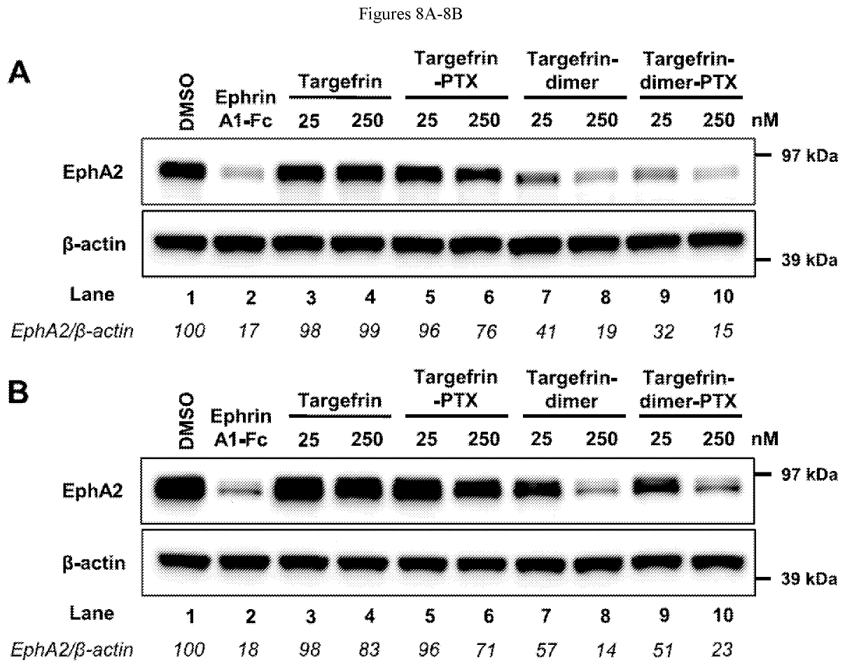

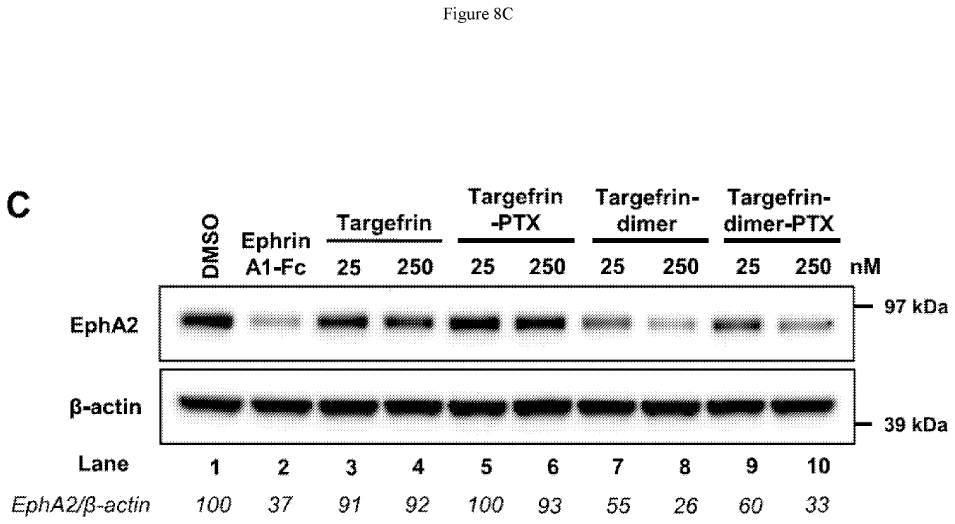

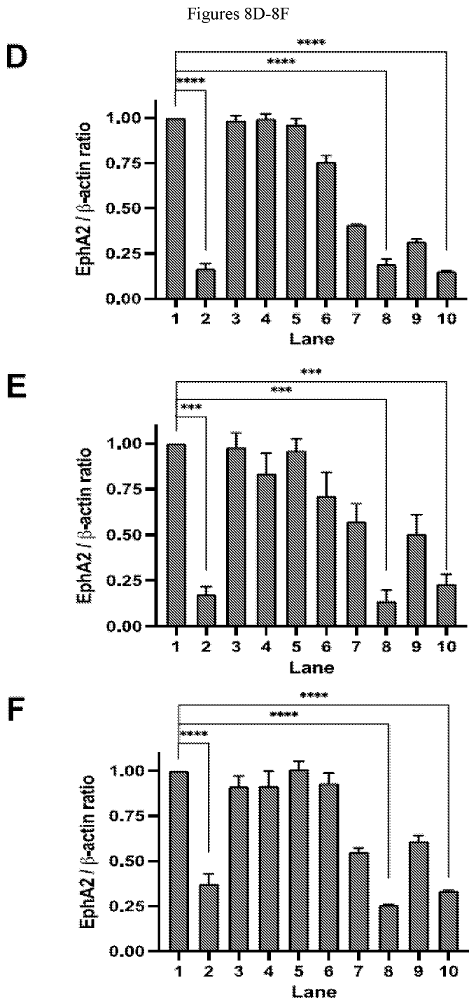

Immunofluorescence microscopy data with BxPC3 cells demonstrated punctuated cytoplasmic fluorescence that co-localized with the lysosomal marker LAMP-1 in the targefrin-dimer-TAMRA treated cells (FIG. 5), confirming an EphA2-specific lysosomal internalization event triggered by the agonistic agents. Indeed, targefrin-dimer-PTX retained its ability to cause EphA2 degradation in all three pancreatic cancer cell lines tested (FIG. 8).

On the contrary, targefrin-monomer-PTX alone did not cause receptor internalization. These data clearly identify targefrin as a potent EphA2-LBD binding agent with antagonistic activity, while targefrin-dimer displayed a similar potent affinity for the isolated EphA2-LBD, but it also displayed potent EphA2 degradation activity in pancreatic cancer cells.

Finally, to determine whether our EphA2 agonistic agents prevent cell motility of pancreatic cancer cells, we conducted a cell migration assay using the scratch wound method as detected with the time-lapsed live cell analysis (IncuCyte S3, Sartorius) of the pancreatic cancer cell line, BxPC3. We reported that in BxPC3, knocking out EphA2 alone resulted in markedly decreased cell migration in this assay.³⁴ Similarly, treatment of BxPC3 cell with increasing concentrations of targefrin-dimer significantly suppressed cell migration (FIG. 6). These data conclude that targefrin and targefrin-dimer are potent antagonistic and agonistic EphA2 agents, respectively.

### In Vivo Pharmacology and Mouse Xenograft Studies

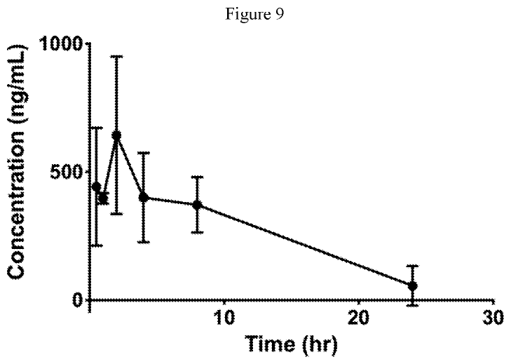

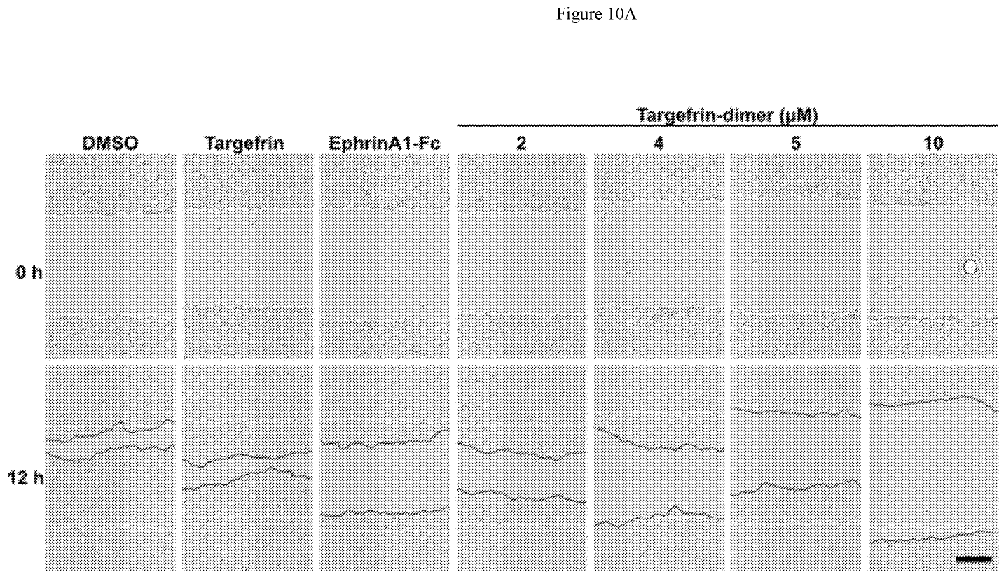

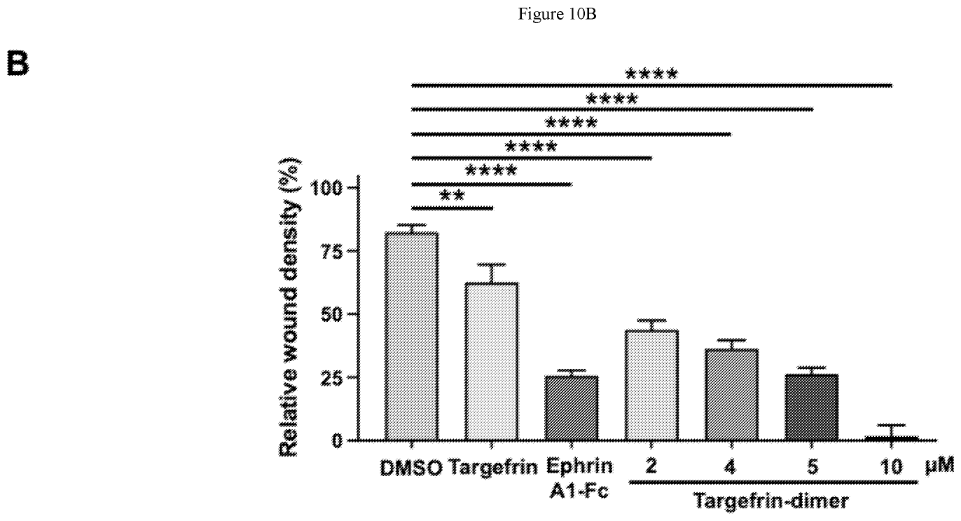

Preliminary pharmacokinetic studies with targefrin-dimer were conducted after administration of a single dose of the agent via the tail vein at 50 mg/Kg, and measuring plasma drug concentration over time (FIG. 9). The data show that the agent reaches a Cₘₐₓ well above the required 100-200 nM to induce EphA2 degradation in cell, and an estimated t½˜15 hr, suggesting that lower drug concentrations could be used for subsequent in vivo efficacy studies. Blood chemistry analyses after this high dose of targefrin-dimer did not reveal any significantly altered values in the blood chemistry panel (e.g., albumin, ALP, ALT, amylase, bilirubin, Ca, P, Na⁺, K⁺, total protein, globulin, creatinine, urea nitrogen, glucose). In an additional preliminary in vivo toxicity study, Balb/C mice were administered with repeated doses (daily for 5 days) of PTX (8 mg/Kg), targefrin-dimer (50 mg/Kg), or targefrin-dimer-PTX (50 mg/Kg); hence, each group was administered with equivalent doses of PTX. 2 of 3 mice receiving PTX were found dead after the 2^{nd} dose, while the remaining mouse looked lethargic and was found dead by day 5. On the contrary, no adverse signs of toxicity were noted in the targefrin-dimer or the targefrin-dimer-PTX treated groups (mice in the latter group looked lethargic after day 1, but recovered). Body weight was monitored during the experiment (Table 6). These preliminary data suggest that targefrin is well tolerated and that it could selectively deliver PTX to EphA2 expressing tumor cells.

TABLE 6
Repeated doses toxicity studies with Targefrin-dimer-PTX versus PTX alone.
Balc/c mice received equimolar doses of PTX or targefrin-dimer-PTX daily
(IV), and body weight were measured daily. FD = found dead. By day
5 all 3 mice in the PTX treated group were found dead. Mice treated with
targefrin-dimer-PTX were lethargic after the first doses but recovered.
No signs of toxicity were noted in the mice treated with targefrin-dimer.

| Mouse | | Body weight (g) | | | | |
|---|---|---|---|---|---|---|
| ID | Treatment | Monday | Tuesday | Wednesday | Thursday | Friday |
| | 50 mg/Kg | | | | | |
| 6 | Targefrin-Dimer- | 26.17 | 25.33 | 25.71 | 25.82 | 26.28 |
| | PTX | | | | | |
| 7 | Targefrin-Dimer- | 25.63 | 24.98 | 25.14 | 24.21 | 24.71 |
| | PTX | | | | | |
| 8 | Targefrin-Dimer- | 26.17 | 25.33 | 26.08 | 25.71 | 25.1 |
| | PTX | | | | | |
| | 8 mg/Kg | | | | | |
| 9 | PTX | 23.77 | 23.22 (FD) | .-----. | .-----. | .-----. |
| 10 | PTX | 23.92 | 23.63 (FD) | .-----. | .-----. | .-----. |
| 11 | PTX | 23.61 | 21.82 | 21.85 | 21.95 | 22.11 (FD) |
| | 50 mg/Kg | | | | | |
| 12 | Targefrin-dimer | 24.48 | 24.14 | 23.83 | 23.21 | 22.94 |
| 13 | Targefrin-dimer | 22.63 | 22.25 | 22.71 | 22.58 | 22.11 |
| 14 | Targefrin-dimer | 24.07 | 23.34 | 23.55 | 23.61 | 23.98 |

Hence, to further assess the ability of the drug conjugates to direct chemotherapy to pancreatic cancer in vivo, we assessed the ability of the agents to suppress tumor growth in a tumor xenograft with MIA PaCa-2 cells. MIA PaCa-2 cells (1.0×10⁷ cells/mouse), in 100 μL PBS, were first injected into the right flank of five nu/nu mice to obtain tumor stock fragments. Subsequently, a 1 mm³ MIA PaCa-2 tumor fragment was grafted in the right flank of each of 25 mice, tumor growth was measured by calipers 18 days after tumor implantation, and mice grouped to receive treatments on days 1, 4, 8, 11, 15, 18. The agents were dissolved in a formulation of 80% PBS, 10% Tween 80, 10% Ethanol. 5 groups received either vehicle control alone, paclitaxel (PTX; 2.5 mg/Kg), targefrin-PTX (10 mg/Kg which is equivalent to 2.5 mg/Kg of PTX), targefrin-dimer-PTX (17 mg/kg which is equivalent to 2.5 mg/Kg of PTX) and a lower dose of targefrin-dimer-PTX (10 mg/kg which is equivalent to 1.5 mg/kg of PTX). Both targefrin-PTX and targefrin-dimer-PTX displayed a significant antitumor effect compared to both the untreated group and the PTX treated group (FIG. 7). Moreover, even the group treated with a sub-stoichiometric dose of PTX became more effective than the PTX treated group (FIG. 7), despite standard deviation on the PTX treated group is too large to assess significance. The data collectively suggests that the agent is capable of delivering the drug to EphA2 expressing tumors.

### Discussion and Conclusions

In recent years, we have witnessed increasing efforts to target EphA2 by various strategies for the development of novel therapeutics.⁴³ These include computational docking strategies,^{31, 44-46} NMR-based screening,^{40,47-48} high-throughput screening,⁴⁹ phage display screening,³⁹ and these efforts resulted in potential small molecule compounds,^{31, 44,8} or EphA2/ephrin antagonists.^{45-46, 49-51,52} However, none of these cited agents are ripe to be used as potential therapeutics. On the contrary, mAbs have been proposed to target EphA2, but did not perform well in the clinic with reduced selectivity or longer half-life that results in accumulation of the agent in undesired tissues.⁵³ Indeed, a very recent phase I clinical study aimed at evaluating the biodistribution of DS-8895a, an anti-EphA2 antibody, in patients with advanced EphA2 positive cancers.⁵⁴ While encouragingly no treatment-related toxicities were reported, DS-8895a had limited therapeutic efficacy likely due to the observed low tumor uptake, causing halting of any further development of DS-8895a.⁵⁴

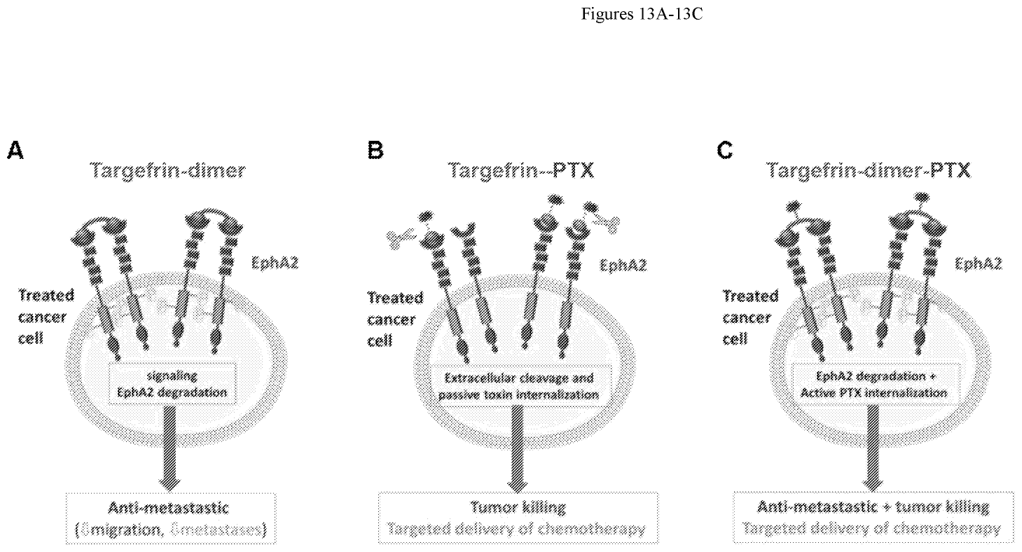

More recently, Bicycle Therapeutics reported on a peptide antagonist binding to EphA2-LBD that binds with a dissociation constant in the low nanomolar range.⁵⁵ The antagonistic agent was conjugated with monomethyl auristatin linked by a cathepsin cleavable linker and it is currently in phase I clinical trials (clinicaltrials.gov/ct2/show/NCT04180371). While this agent holds great promise for the first translation of an EphA2 targeting agent into a possible therapeutic, targefrin and targefrin-dimer offer valid alternative strategies to the Bicycle Therapeutics agent. First, targefrin has a similar affinity as the Bicycle Therapeutics compound for EphA2 but possesses reduced molecular weight, presumably enhancing its tissue penetration; second, targefrin-dimer induces active internalization of the receptor functions as an effective EphA2 degrader; hence, it could be deployed as an effective EphA2-based therapeutic to suppress cell migration (FIG. 6), as an alternative to agonistic antibodies. Hence, we envision that targefrin-dimer could be deployed as an EphA2 degrader to suppress the metastatic behavior of cancer cells (FIG. 13), as we had recently demonstrated with earlier agent 135H12 in an orthotopic models of prostate cancer.³³

Moreover, in drug conjugates, the active internalization induced by targefrin-dimer does not require extracellular linker cleavage and passive diffusion of the cargo, potentially increasing the distribution of the chemotherapeutic agent to EphA2 expressing tumor cells. We observed that an earlier dimeric EphA2 targeting agent conjugated with paclitaxel induced a marked reduction of circulating tumor cells in tumor bearing mice.⁸ Here, we observed that a sub-therapeutic dose of paclitaxel is effective in reducing tumor volume when conjugated to both monomeric and dimeric versions of targefrin (FIG. 7).

Together with our reported preliminary toxicity and pharmacokinetics studies, we suggest that the dimer could be deployed as single agent or in combination with standard of care to suppress EphA2 in cancer cells. In addition, preliminary studies with drug conjugates should encourage further evaluations with such agents, particularly when the conjugation is to targefrin-dimer exploiting the active internalization provided by the agent to EphA2-overexpressing tumors.

In conclusion, the agents reported herein open the way to a wide range of opportunities for the development of EphA2-targeting therapeutics, ranging from more effective PDCs to the development of diagnostics, or for devising more effective combination therapies targeting tumor metastases.

### Experimental Section.

### Chemistry

General. All reagents and solvents were obtained from commercial sources, including fmoc-protected amino acids and resins for solid phase synthesis. All the peptides were synthesized in house by standard microwave assisted Fmoc peptide synthesis protocols on Rink amide resin using a Liberty Blue peptide synthesizer (CEM). For each coupling reaction, 3 equivalents of Fmoc-AA, 3 equivalents of DIC, and 1 equivalent of OximaPure in 4.5 mL of DMF were used. The coupling reaction was allowed to proceed for 5 min at 90° C. in the microwave reactor. Fmoc deprotection was performed by treating the resin-bound peptide with 20% N-Methylpiperidine in DMF (2× 3 mL) for 3 min at 90° C. Peptides were cleaved from the resin with a cleavage cocktail containing TFA/TIS/H₂O/phenol (94:2:2:2) for 5 h (see Example 3). The cleaving solution was filtered from the resin, and the peptides were precipitated in Et₂O, centrifuged, and dried in a high vacuum. Solution ¹H NMR was used to check concentration and spectra were recorded on Bruker Avance III 700 MHz. High resolution mass spectral data were acquired on an Agilent LC-TOF instrument. RP-HPLC purifications were performed on a JASCO preparative system equipped with a PDA detector and a fraction collector controlled by a ChromNAV system (JASCO) on a XTerra C18 10μ 10×250 mm (Waters). Purity of tested compounds was assessed by HPLC using an Atlantis T3 3 μm 4.6×150 mm² column (H2O/ACN gradient from 5 to 100% in 45 min). All compounds have a purity >95%

Preparation of dimeric agents and targefrin-dimer. The preparation of the dimeric agents was done following the procedure described above, but doubling the equivalents for each coupling and introducing an Fmoc-Lys(Fmoc)-OH as the first amino acid of the sequence as illustrated in Example 4.

Preparation of targefrin-motif and targefrin-dimer-motif. For the preparation of the targefrin-motif and targefrin-dimer-motif we introduced as first amino acid, coupled to a Rink Amide resin, a Fmoc-Lys(ivDde)-OH amino acid. Subsequently the peptides have been growth following a solid-phase synthetic scheme similar to what we previously described. At synthesis completed, the fully protected peptide on a Rink amide resin was treated with 4% solution of Hydrazine in DMF (3× 5 mL, each 30 min) to remove the ivDde protecting group and was subsequently washed with DMF (3×5 mL). This was followed by a coupling with 3 equiv. of 5-hexynoic acid in the presence of 3 equiv. of HATU, 3 equiv. of OximaPure, and 5 equiv of DIPEA in 1 mL of DMF for 1 hour at room temperature. The resin then was washed with DMF (3×5 mL) and DCM (3×5 mL), dried under vacuum and cleaved with a cleavage cocktail containing TFA/TIS/H2O/phenol (94:2:2:2) for h. Synthetic scheme for targefrin-motif and targefrin-dimer-motif are reported in Example 5 and Example 6 respectively.

Preparation of targefrin-PTX, targefrin-dimer-PTX, and targefrin-dimer-TAMRA. Crude targefrin-motif was dissolved with 1 equiv. of PTX-Azide into 4 mL of a 4:1 DMSO:water stirring solution in presence of 50 μL of CuSO, 1 M and 50 μL of Sodium Ascorbate 1M, at room temperature for 48 h (Example 7). Targefrin-dimer-PTX was obtain as described above, but using crude targefrin-dimer-motif as starting point (Example 8). Targefrin-dimer-TAMRA was obtained as described before but using 1 equiv. of 5-TAMRA-azide instead of PTX-azide (Example 17). Mass spectrometry data for representative peptides synthesized are reported in Table 1.

### Isothermal Titration Calorimetry (ITC) Measurements.

To obtain information about the dissociation constant (K_{d}) and thermodynamics of binding of our EphA2 targeting agents, we tested the compounds by Isothermal titration calorimetry (ITC) performed using the Affinity ITC Autosampler from TA Instruments (New Castle, DE) against EphA2-LBD. The titrations were performed in a reverse fashion by titrating the protein into the ligand solution. All titrations were performed dissolving both the agents and the targeting protein in 25 mM Tris at pH 7.5, 150 mM NaCl, at 25° C. with a final DMSO concentration of 1%. The syringe was filled with a 200 μM solution of EphA2-LBD, EphA3-LBD Chimera, or EphA4-LBD, performing 20 injections of 2.5 μL each into the cell containing a 10 μM solution of the compounds. The injections were made at 200 s intervals with a stirring speed of 75 rpm. The solutions were kept in the autosampler at 4° C. The analysis of the data was performed by the NanoAnalyze software (TA Instruments, New Castle, DE) and subsequently exported into Microsoft Excel.

### DELFIA Displacement Assays

To test the activity of the dimeric and monomeric agents, a solution of 100 μL of 1 μM of 123B9-Biotin1 or 100 nM of agent PiperazineAcAcid-YSA-(2MeBip)-PDS-Chg-PFRP-GK (Biotin LC) was added to each well of 96-well streptavidin-coated plates, respectively, and incubated for 2 h. Plates were then washed 3 times. Subsequently, a mixture containing 11 μL of EphA2 protein and a serial dilution of the test compounds was added to each well and incubated with a solution containing 89 μL of Eu-N1-labeled anti-6x-His antibody (PerkinElmer) for 1 h. At the end of the incubation period, plates were washed 3 times and incubated with DELFIA enhancement solution (PerkinElmer) for 10 min. The final concentrations of EphA2 protein used to test the activity of dimeric and monomeric agents were 71.2 nM and 10 nM, respectively. The antibody concentrations in a solution of 89 μL used to test the dimeric and monomeric agents were 4.17 and 3.13 nM, respectively. EphA2 protein, biotinylated peptides and antibody were prepared in DELFIA assay buffer (PerkinElmer). Fluorescence measurements were taken with the VICTOR X5 microplate reader (ex/em of 340/615 nm), normalized to DMSO wells, and reported as percent inhibition. Prism 9 (GraphPad) was used to calculate IC₅₀ values.

### Cell Lines, Cell Culture and Antibodies

BxPC3, MIA PaCa-2, and PANC-1 cell lines were purchased from the American Type Culture Collection (ATCC). BxPC3 and PANC-1 cells were cultured in RPMI-1640 medium and DMEM medium, respectively, and supplemented with 10% fetal bovine serum (FBS). MIA PaCa-2 cells were cultured in DMEM medium supplemented with 10% FBS and 2.5% horse serum. Cells were maintained at 37° C. in a humidified incubator with 5% CO₂. Anti-EphA2 antibody (#374400), HRP-conjugated goat anti-mouse secondary antibody (#31432) and Alexa Fluor 488-conjugated goat anti-rabbit secondary antibody (#A-11034) were purchased from ThermoFisher Scientific. Anti-β-actin antibody (#sc-69879) was purchased from Santa Cruz Biotechnology and anti-LAMP1 antibody (#9091) was purchased from Cell Signaling Technology.

### Immunofluorescence

BxPC3 cells were plated on the coverslips overnight. Cells were serum starved for 1 h and treated with 100 nM targefrin-dimer-TAMRA for 0, 30 and 60 min. Cells were then fixed with 4% paraformaldehyde for 20 min, permeabilized with 0.2% Triton X-100 for 5 min, blocked with 10% goat serum for 1 h and incubated with an anti-LAMP1 antibody overnight at 4° C. followed by an incubation with an anti-rabbit secondary antibody conjugated with Alexa Fluor™ 488 for 1 h at room temperature. VECTASHIELD antifade mounting medium containing DAPI (Vector Laboratories) was added to the coverslips to stain for the nuclei. Images were then acquired using a Zeiss Axiovert 200M fluorescence deconvolution microscope and processed with a SlideBook software version 6 (Intelligent Imaging Innovations).

### Immunoblotting

After treatments, cells were lysed on ice with a lysis buffer (20 mM Tris, pH 7.4, 120 mM NaCl, 1% Triton X-100, 0.5% sodium deoxycholate, 0.1% SDS, 1% IGEPAL, and 5 mM EDTA, supplemented with a protease inhibitor cocktail and PhosSTOP (Sigma-Aldrich). Lysates were then centrifuged at 16,000×g for 20 min at 4° C. and supernatants were collected. Protein determination was done using the Pierce BCA Protein Assay Kit (ThermoFisher Scientific) according to the manufacturer's protocol. Samples were prepared and loaded onto 4-12% NuPAGE Bis-Tris precast gels prior to being transferred onto PVDF membranes. Blots were blocked with 5% nonfat milk for 1 h at room temperature and incubated with monoclonal EphA2 or actin antibodies overnight at 4° C. followed by an incubation with an anti-mouse HRP conjugated antibody for 1 h at room temperature. The Clarity Western ECL kit (BIO-RAD) was added to the blots, and images were captured with the ChemiDoc imaging system (BIO-RAD) and analyzed using ImageJ software.

### Cell Migration Assays

BxPC3 cells were seeded in the IncuCyte® ImageLock 96-well plates (Sartorius) so that they were approximately at 95-100% confluency by the time of the treatment. Wounds were then made on a monolayer of cells using the WoundMaker™ (Sartorius) followed by two washes with PBS. Cells were subsequently treated with 2 μg/mL ephrinA1-Fc (R&D Systems) or the test agents and the plates were imaged every 3 h with the IncuCyte® S3 live-cell analysis system (Sartorius). The percentage relative wound density was quantified using the IncuCytek cell migration software module.

### In Vivo Pharmacokinetics, Toxicity, Xenograft Studies

The in vivo efficacy experiment was conducted at AntiCancer, Inc. (San Diego). For the xenograft study, 35 male nu/nu mice (AntiCancer Inc, San Diego), 8-10 weeks of age, were used, consisting of 25 mice for randomization and 10 extra mice. All mice were kept in a barrier facility on a high efficacy particulate air (HEPA)-filtered rack under standard conditions of 12 h light/dark cycles. Animal studies were performed with an AntiCancer Institutional Animal Care and Use Committee (IACUC)-protocol specially approved for this study and in accordance with the principles and procedures outlined in the National Institutes of Health Guide for the Care and Use of Animals under Assurance Number A3873-1. Autoclaved, acidified water (pH 2.5-3) was supplied ad libitum to all animals. Cryogenic vials containing MIA PaCa-2 pancreatic cancer cells were thawed from liquid nitrogen storage and expanded for in vitro cell culture to prepare subcutaneous stock tumor for subsequent flank tumor-fragment implantation. MIA PaCa-2 cells were maintained in DMEM supplemented with 10% heat-inactivated fetal bovine serum and 1% penicillin and cultured at 37° C. in a 5% CO₂ incubator. Hence, MIA PaCa-2 cells (1.0×10⁷ cells/mouse), in 100 μL PBS, were injected into the right flank of five male nu/nu mice. After mice were put under anesthesia using a ketamine solution, an approximate 5 mm incision was made on the back of nude mice. After making a space under the skin of the right flank, a 1 mm³ MIA PaCa-2 tumor fragment, prepared from stock, was inserted. The incision was closed with a 5-0 PDS-II suture. 18 days after tumor implantation (Day 0), tumors were measured by calipers, using the formula: (Tumor volume)=(Length)×(Width)×(Width)×½. 25 out of 35 mice were randomized into five treatment groups of 5 mice, with no significant difference in tumor volume between the groups. All treatment agents (dissolved in 100 μl formulation comprised of 80% PBS, 10% Tween 80, 10% Ethanol) were administered by tail vein injection twice per week for 3 weeks, for a total of 6 injections. Treatment was begun the day after randomization (Day 1) and mice received agents or vehicle control on Days 1, 4, 8, 11, 15, 18. Tumor volume and body weight were measured weekly. The study was terminated 22 days after the initiation of the treatment.

### Molecular Modeling

Molecular models were analyzed using MOE 2022.02 (Chemical Computing Group). The model of targefrin in complex with EphA2-LBD, was obtain modifying and properly minimizing the crystal structure of our previous agent with EphA2-LBD (PDB-ID 6B9L).

The content of C. Baggio, et al., J. Med. Chem. 2022, 65, 22, 15443-15456 is incorporated by reference herein.

### References in Example 18

1. Gambini, L.; Salem, A. F.; Udompholkul, P.; Tan, X. F.; Baggio, C.; Shah, N.; Aronson, A.; Song, J.; Pellecchia, M., Structure-Based Design of Novel Epha2 Agonistic Agents with Nanomolar Affinity in Vitro and in Cell. ACS Chem Biol 2018, 13 (9), 2633-2644.
2. Duxbury, M. S.; Ito, H.; Zinner, M. J.; Ashley, S. W.; Whang, E. E., Ligation of Epha2 by Ephrin Al-Fc Inhibits Pancreatic Adenocarcinoma Cellular Invasiveness. Biochemical and biophysical research communications 2004, 320 (4), 1096-1102.
3. Duxbury, M. S.; Ito, H.; Zinner, M. J.; Ashley, S. W.; Whang, E. E., Epha2: A Determinant of Malignant Cellular Behavior and a Potential Therapeutic Target in Pancreatic Adenocarcinoma. Oncogene 2004, 23 (7), 1448-1456.
4. Mudali, S. V.; Fu, B.; Lakkur, S. S.; Luo, M.; Embuscado, E. E.; Iacobuzio-Donahue, C. A., Patterns of Epha2 Protein Expression in Primary and Metastatic Pancreatic Carcinoma and Correlation with Genetic Status. Clinical & experimental metastasis 2006, 23 (7-8), 357-365.
5. Hess, A. R.; Seftor, E. A.; Gardner, L. M.; Carles-Kinch, K.; Schneider, G. B.; Seftor, R. E.; Kinch, M. S.; Hendrix, M. J., Molecular Regulation of Tumor Cell Vasculogenic Mimicry by Tyrosine Phosphorylation: Role of Epithelial Cell Kinase (Eck/Epha2). Cancer research 2001, 61 (8), 3250-3255.
6. Walker-Daniels, J.; Coffman, K.; Azimi, M.; Rhim, J. S.; Bostwick, D. G.; Snyder, P.; Kerns, B. J.; Waters, D. J.; Kinch, M. S., Overexpression of the Epha2 Tyrosine Kinase in Prostate Cancer. The Prostate 1999, 41 (4), 275-280.
7. Zeng, G.; Hu, Z.; Kinch, M. S.; Pan, C. X.; Flockhart, D. A.; Kao, C.; Gardner, T. A.; Zhang, S.; Li, L.; Baldridge, L. A.; Koch, M. O.; Ulbright, T. M.; Eble, J. N.; Cheng, L., High-Level Expression of Epha2 Receptor Tyrosine Kinase in Prostatic Intraepithelial Neoplasia. The American journal of pathology 2003, 163 (6), 2271-2276.
8. Salem, A. F.; Wang, S.; Billet, S.; Chen, J. F.; Udompholkul, P.; Gambini, L.; Baggio, C.; Tseng, H. R.; Posadas, E. M.; Bhowmick, N. A.; Pellecchia, M., Reduction of Circulating Cancer Cells and Metastases in Breast-Cancer Models by a Potent Epha2-Agonistic Peptide-Drug Conjugate. J Med Chem 2018, 61 (5), 2052-2061.
9 Zhao, P.; Jiang, D.; Huang, Y.; Chen, C., Epha2: A Promising Therapeutic Target in Breast Cancer. J Genet Genomics 2021, 48 (4), 261-267.
10. Zhou, L.; Lu, X.; Zhang, B.; Shi, Y.; Li, Z., Epha2 as a New Target for Breast Cancer and Its Potential Clinical Application. Int J Clin Exp Pathol 2021, 14 (4), 484-492.
11. Miyazaki, T.; Kato, H.; Fukuchi, M.; Nakajima, M.; Kuwano, H., Epha2 Overexpression Correlates with Poor Prognosis in Esophageal Squamous Cell Carcinoma. International journal of cancer. Journal international du cancer 2003, 103 (5), 657-663.
12. Ogawa, K.; Pasqualini, R.; Lindberg, R. A.; Kain, R.; Freeman, A. L.; Pasquale, E. B., The Ephrin-Al Ligand and Its Receptor, Epha2, Are Expressed During Tumor Neovascularization. Oncogene 2000, 19 (52), 6043-6052.
13. Margaryan, N. V.; Strizzi, L.; Abbott, D. E.; Seftor, E. A.; Rao, M. S.; Hendrix, M. J.; Hess, A. R., Epha2 as a Promoter of Melanoma Tumorigenicity. Cancer biology & therapy 2009, 8 (3), 279-288.
14. Abraham, S.; Knapp, D. W.; Cheng, L.; Snyder, P. W.; Mittal, S. K.; Bangari, D. S.; Kinch, M.; Wu, L.; Dhariwal, J.; Mohammed, S. I., Expression of Epha2 and Ephrin a-1 in Carcinoma of the Urinary Bladder. Clinical cancer research: an official journal of the American Association for Cancer Research 2006, 12 (2), 353-360.
15. Wang, L. F.; Fokas, E.; Bieker, M.; Rose, F.; Rexin, P.; Zhu, Y.; Pagenstecher, A.; Engenhart-Cabillic, R.; An, H. X., Increased Expression of Epha2 Correlates with Adverse Outcome in Primary and Recurrent Glioblastoma Multiforme Patients. Oncology reports 2008, 19 (1), 151-156.
16. Wykosky, J.; Gibo, D. M.; Stanton, C.; Debinski, W., Epha2 as a Novel Molecular Marker and Target in Glioblastoma Multiforme. Molecular cancer research: MCR 2005, 3 (10), 541-551.
17. Binda, E.; Visioli, A.; Giani, F.; Lamorte, G.; Copetti, M.; Pitter, K. L.; Huse, J. T.; Cajola, L.; Zanetti, N.; DiMeco, F.; De Filippis, L.; Mangiola, A.; Maira, G.; Anile, C.; De Bonis, P.; Reynolds, B. A.; Pasquale, E. B.; Vescovi, A. L., The Epha2 Receptor Drives Self-Renewal and Tumorigenicity in Stem-Like Tumor-Propagating Cells from Human Glioblastomas. Cancer cell 2012, 22 (6), 765-780.
18. Faoro, L.; Singleton, P. A.; Cervantes, G. M.; Lennon, F. E.; Choong, N. W.; Kanteti, R.; Ferguson, B. D.; Husain, A. N.; Tretiakova, M. S.; Ramnath, N.; Vokes, E. E.; Salgia, R., Epha2 Mutation in Lung Squamous Cell Carcinoma Promotes Increased Cell Survival, Cell Invasion, Focal Adhesions, and Mammalian Target of Rapamycin Activation. The Journal of biological chemistry 2010, 285 (24), 18575-18585.
19. Merritt, W. M.; Thaker, P. H.; Landen, C. N., Jr.; Deavers, M. T.; Fletcher, M. S.; Lin, Y. G.; Han, L. Y.; Kamat, A. A.; Schmandt, R.; Gershenson, D. M.; Kinch, M. S.; Sood, A. K., Analysis of Epha2 Expression and Mutant P53 in Ovarian Carcinoma. Cancer biology & therapy 2006, 5 (10), 1357-1360.
20. Yuan, W. J.; Ge, J.; Chen, Z. K.; Wu, S. B.; Shen, H.; Yang, P.; Hu, B.; Zhang, G. W.; Chen, Z. H., Over-Expression of Epha2 and Ephrina-1 in Human Gastric Adenocarcinoma and Its Prognostic Value for Postoperative Patients. Digestive diseases and sciences 2009, 54 (11), 2410-2417.
21. Takahashi, Y.; Itoh, M.; Nara, N.; Tohda, S., Effect of Eph-Ephrin Signaling on the Growth of Human Leukemia Cells. Anticancer Res 2014, 34 (6), 2913-2918.
22. Trinidad, E. M.; Zapata, A. G.; Alonso-Colmenar, L. M., Eph-Ephrin Bidirectional Signaling Comes into the Context of Lymphocyte Transendothelial Migration. Cell Adh Migr 2010, 4 (3), 363-367.
23. Alonso, C. L.; Trinidad, E. M.; de Garcillan, B.; Ballesteros, M.; Castellanos, M.; Cotillo, I.; Munoz, J. J.; Zapata, A. G., Expression Profile of Eph Receptors and Ephrin Ligands in Healthy Human B Lymphocytes and Chronic Lymphocytic Leukemia B-Cells. Leuk Res 2009, 33 (3), 395-406.
24. Guan, M.; Liu, L.; Zhao, X.; Wu, Q.; Yu, B.; Shao, Y.; Yang, H.; Fu, X.; Wan, J.; Zhang, W., Copy Number Variations of Epha3 Are Associated with Multiple Types of Hematologic Malignancies. Clin Lymphoma Myeloma Leuk 2011, 11 (1), 50-53.
25. Amato, K. R.; Wang, S.; Hastings, A. K.; Youngblood, V. M.; Santapuram, P. R.; Chen, H.; Cates, J. M.; Colvin, D. C.; Ye, F.; Brantley-Sieders, D. M.; Cook, R. S.; Tan, L.; Gray, N. S.; Chen, J., Genetic and Pharmacologic Inhibition of Epha2 Promotes Apoptosis in Nsclc. J Clin Invest 2014, 124 (5), 2037-2049.
26. Amato, K. R.; Wang, S.; Tan, L.; Hastings, A. K.; Song, W.; Lovly, C. M.; Meador, C. B.; Ye, F.; Lu, P.; Balko, J. M.; Colvin, D. C.; Cates, J. M.; Pao, W.; Gray, N. S.; Chen, J., Epha2 Blockade Overcomes Acquired Resistance to Egfr Kinase Inhibitors in Lung Cancer. Cancer research 2016, 76 (2), 305-318.
27. Miao, B.; Ji, Z.; Tan, L.; Taylor, M.; Zhang, J.; Choi, H. G.; Frederick, D. T.; Kumar, R.; Wargo, J. A.; Flaherty, K. T.; Gray, N. S.; Tsao, H., Epha2 Is a Mediator of Vemurafenib Resistance and a Novel Therapeutic Target in Melanoma. Cancer Discov 2015, 5 (3), 274-287.
28. Heinzlmeir, S.; Kudlinzki, D.; Sreeramulu, S.; Klaeger, S.; Gande, S. L.; Linhard, V.; Wilhelm, M.; Qiao, H.; Helm, D.; Ruprecht, B.; Saxena, K.; Medard, G.; Schwalbe, H.; Kuster, B., Chemical Proteomics and Structural Biology Define Epha2 Inhibition by Clinical Kinase Drugs. ACS Chem Biol 2016, 11 (12), 3400-3411.
29. Petty, A.; Idippily, N.; Bobba, V.; Geldenhuys, W. J.; Zhong, B.; Su, B.; Wang, B., 29. Design and Synthesis of Small Molecule Agonists of Epha2 Receptor. Eur J Med Chem 2018, 143, 1261-1276.
30. Hasegawa, J.; Sue, M.; Yamato, M.; Ichikawa, J.; Ishida, S.; Shibutani, T.; Kitamura, M.; Wada, T.; Agatsuma, T., Novel Anti-Epha2 Antibody, Ds-8895a for Cancer Treatment. Cancer biology & therapy 2016, 17 (11), 1158-1167.
31. Petty, A.; Myshkin, E.; Qin, H.; Guo, H.; Miao, H.; Tochtrop, G. P.; Hsieh, J. T.; Page, P.; Liu, L.; Lindner, D. J.; Acharya, C.; Mackerell, A. D., Jr.; Ficker, E.; Song, J.; Wang, B., A Small Molecule Agonist of Epha2 Receptor Tyrosine Kinase Inhibits Tumor Cell Migration in Vitro and Prostate Cancer Metastasis in Vivo. PloS one 2012, 7 (8), e42120.
32. Singh, D. R.; Kanvinde, P.; King, C.; Pasquale, E. B.; Hristova, K., The Epha2 Receptor Is Activated through Induction of Distinct, Ligand-Dependent Oligomeric Structures. Commun Biol 2018, 1, 15.
33. Salem, A. F.; Gambini, L.; Billet, S.; Sun, Y.; Oshiro, H.; Zhao, M.; Hoffman, R. M.; Bhowmick, N. A.; Pellecchia, M., Prostate Cancer Metastases Are Strongly Inhibited by Agonistic Epha2 Ligands in an Orthotopic Mouse Model. Cancers (Basel) 2020, 12 (10).
34. Salem, A. F.; Gambini, L.; Udompholkul, P.; Baggio, C.; Pellecchia, M., Therapeutic Targeting of Pancreatic Cancer Via Epha2 Dimeric Agonistic Agents. Pharmaceuticals (Basel) 2020, 13 (5).
35. Quinn, B. A.; Wang, S.; Barile, E.; Das, S. K.; Emdad, L.; Sarkar, D.; De, S. K.; Morvaridi, S. K.; Stebbins, J. L.; Pandol, S. J.; Fisher, P. B.; Pellecchia, M., Therapy of Pancreatic Cancer Via an Epha2 Receptor-Targeted Delivery of Gemcitabine. Oncotarget 2016, 7 (13), 17103-17110.
36. Wang, S.; Placzek, W. J.; Stebbins, J. L.; Mitra, S.; Noberini, R.; Koolpe, M.; Zhang, Z.; Dahl, R.; Pasquale, E. B.; Pellecchia, M., Novel Targeted System to Deliver Chemotherapeutic Drugs to Epha2-Expressing Cancer Cells. J Med Chem 2012, 55 (5), 2427-2436.
37. Wu, B.; Wang, S.; De, S. K.; Barile, E.; Quinn, B. A.; Zharkikh, I.; Purves, A.; Stebbins, J. L.; Oshima, R. G.; Fisher, P. B.; Pellecchia, M., Design and Characterization of Novel Epha2 Agonists for Targeted Delivery of Chemotherapy to Cancer Cells. Chem Biol 2015, 22 (7), 876-887.
38. Barile, E.; Wang, S.; Das, S. K.; Noberini, R.; Dahl, R.; Stebbins, J. L.; Pasquale, E. B.; Fisher, P. B.; Pellecchia, M., Design, Synthesis and Bioevaluation of an Epha2 Receptor-Based Targeted Delivery System. ChemMedChem 2014, 9 (7), 1403-1412.
39. Mitra, S.; Duggineni, S.; Koolpe, M.; Zhu, X.; Huang, Z.; Pasquale, E. B., Structure-Activity Relationship Analysis of Peptides Targeting the Epha2 Receptor. Biochemistry 2010, 49 (31), 6687-6695.
40. Wu, B.; Barile, E.; De, S. K.; Wei, J.; Purves, A.; Pellecchia, M., High-Throughput Screening by Nuclear Magnetic Resonance (Hts by Nmr) for the Identification of Ppis Antagonists. Curr Top Med Chem 2015, 15 (20), 2032-2042.
41. Udompholkul, P.; Baggio, C.; Gambini, L.; Sun, Y.; Zhao, M.; Hoffman, R. M.; Pellecchia, M., Effective Tumor Targeting by Epha2-Agonist-Biotin-Streptavidin Conjugates. Molecules 2021, 26 (12).
42. Duggineni, S.; Mitra, S.; Lamberto, I.; Han, X.; Xu, Y.; An, J.; Pasquale, E. B.; Huang, Z., Design and Synthesis of Potent Bivalent Peptide Agonists Targeting the Epha2 Receptor. ACS Med Chem Lett 2013, 4 (3).
43. Lodola, A.; Giorgio, C.; Incerti, M.; Zanotti, I.; Tognolini, M., Targeting Eph/Ephrin System in Cancer Therapy. Eur J Med Chem 2017, 142, 152-162.
44. Petty, A.; Idippily, N.; Bobba, V.; Geldenhuys, W. J.; Zhong, B.; Su, B.; Wang, B., Design and Synthesis of Small Molecule Agonists of Epha2 Receptor. Eur J Med Chem 2017.
45. Hassan-Mohamed, I.; Giorgio, C.; Incerti, M.; Russo, S.; Pala, D.; Pasquale, E. B.; Zanotti, I.; Vicini, P.; Barocelli, E.; Rivara, S.; Mor, M.; Lodola, A.; Tognolini, M., Unipr129 Is a Competitive Small Molecule Eph-Ephrin Antagonist Blocking in Vitro Angiogenesis at Low Micromolar Concentrations. Br J Pharmacol 2014, 171 (23), 5195-5208.
46. Tognolini, M.; Incerti, M.; Pala, D.; Russo, S.; Castelli, R.; Hassan-Mohamed, I.; Giorgio, C.; Lodola, A., Target Hopping as a Useful Tool for the Identification of Novel Epha2 Protein-Protein Antagonists. ChemMedChem 2014, 9 (1), 67-72.
47. Wu, B.; De, S. K.; Kulinich, A.; Salem, A. F.; Koeppen, J.; Wang, R.; Barile, E.; Wang, S.; Zhang, D.; Ethell, I.; Pellecchia, M., Potent and Selective Epha4 Agonists for the Treatment of Als. Cell Chem Biol 2017, 24 (3), 293-305.
48. Wu, B.; Zhang, Z.; Noberini, R.; Barile, E.; Giulianotti, M.; Pinilla, C.; Houghten, R. 48. A.; Pasquale, E. B.; Pellecchia, M., Hts by Nmr of Combinatorial Libraries: A Fragment-Based Approach to Ligand Discovery. Chem Biol 2013, 20 (1), 19-33.
49. Giorgio, C.; Incerti, M.; Corrado, M.; Rusnati, M.; Chiodelli, P.; Russo, S.; Callegari, D.; Ferlenghi, F.; Ballabeni, V.; Barocelli, E.; Lodola, A.; Tognolini, M., Pharmacological Evaluation of New Bioavailable Small Molecules Targeting Eph/Ephrin Interaction. Biochem Pharmacol 2017.
50. Incerti, M.; Tognolini, M.; Russo, S.; Pala, D.; Giorgio, C.; Hassan-Mohamed, I.; Noberini, R.; Pasquale, E. B.; Vicini, P.; Piersanti, S.; Rivara, S.; Barocelli, E.; Mor, M.; Lodola, A., Amino Acid Conjugates of Lithocholic Acid as Antagonists of the Epha2 Receptor. J Med Chem 2013, 56 (7), 2936-2947.
51. Castelli, R.; Tognolini, M.; Vacondio, F.; Incerti, M.; Pala, D.; Callegari, D.; Bertoni, S.; Giorgio, C.; Hassan-Mohamed, I.; Zanotti, I.; Bugatti, A.; Rusnati, M.; Festuccia, C.; Rivara, S.; Barocelli, E.; Mor, M.; Lodola, A., Delta (5)-Cholenoyl-Amino Acids as Selective and Orally Available Antagonists of the Eph-Ephrin System. Eur J Med Chem 2015, 103, 312-324.
52. Tandon, M.; Vemula, S. V.; Mittal, S. K., Emerging Strategies for Epha2 Receptor Targeting for Cancer Therapeutics. Expert Opin Ther Targets 2011, 15 (1), 31-51.
53. Annunziata, C. M.; Kohn, E. C.; LoRusso, P.; Houston, N. D.; Coleman, R. L.; Buzoianu, M.; Robbie, G.; Lechleider, R., Phase 1, Open-Label Study of Medi-547 in Patients with Relapsed or Refractory Solid Tumors. Invest New Drugs 2013, 31 (1), 77-84.
54. Gan, H. K.; Parakh, S.; Lee, F. T.; Tebbutt, N. C.; Ameratunga, M.; Lee, S. T.; O'Keefe, G. J.; Gong, S. J.; Vanrenen, C.; Caine, J.; Giovannetti, M.; Murone, C.; Scott, F. E.; Guo, N.; Burvenich, I. J. G.; Paine, C.; Macri, M. J.; Kotsuma, M.; Senaldi, G.; Venhaus, R.; Scott, A. M., A Phase 1 Safety and Bioimaging Trial of Antibody Ds-8895a against Epha2 in Patients with Advanced or Metastatic Epha2 Positive Cancers. Invest New Drugs 2022, 40 (4), 747-755.
55. Mudd, G. E.; Brown, A.; Chen, L.; van Rietschoten, K.; Watcham, S.; Teufel, D. P.; Pavan, S.; Lani, R.; Huxley, P.; Bennett, G. S., Identification and Optimization of Epha2-Selective Bicycles for the Delivery of Cytotoxic Payloads. J Med Chem 2020, 63 (8), 4107-4116.

### Example 19

TABLE 7
Tested agents with 4-phenyl-L-phenylalanine fixed in position 4 and relative K_{d} values
(nM) from ITC.

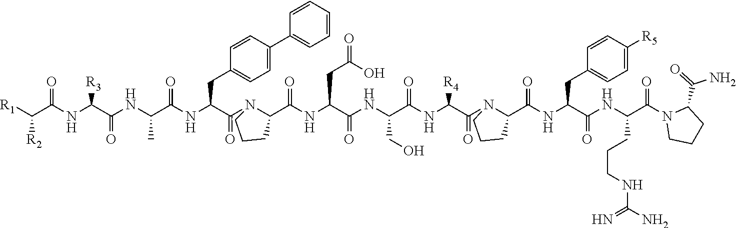

| | | | | | | K_{d} (nM)- |
|---|---|---|---|---|---|---|
| ID | R₁ | R₂ | R₃ | R₄ | R₅ | ITC |
| 152B8 (31) | 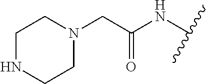 [SMILES: *NC(=O)CN1CCNCC1] | 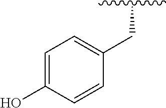 | 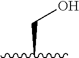 | 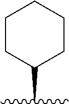 | H | 54 |
| 152D7 (32) | 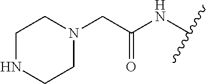 [SMILES: *NC(=O)CN1CCNCC1] | 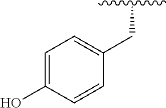 | 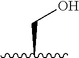 | 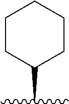 | Cl | 68 |
| 152D9 (33) | 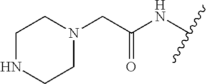 [SMILES: *NC(=O)CN1CCNCC1] | 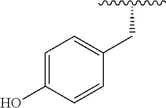 | 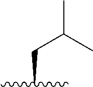 | 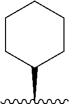 | H | 39 |
| 152D10 (34) | 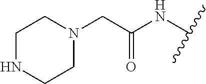 [SMILES: *NC(=O)CN1CCNCC1] | 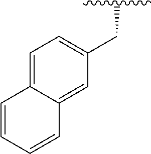 | 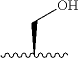 | 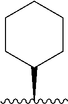 | H | 74 |

TABLE 8
Tested agents with 4-(2-Methylphenyl)-L-phenylalanine fixed in position 4 and relative K_{d}
values (nM) from ITC.

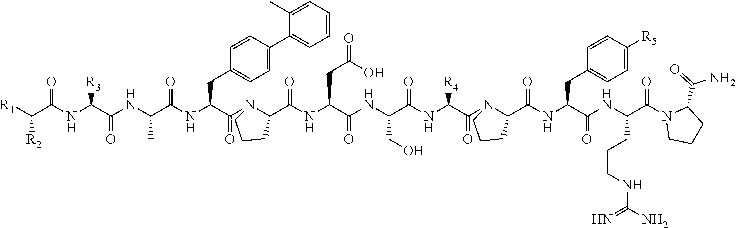

| | | | | | | K_{d} (nM)- | IC50 |
|---|---|---|---|---|---|---|---|
| ID | R₁ | R₂ | R₃ | R₄ | R₅ | ITC | (nM) |
| 152B12 (35) | 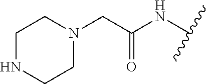 [SMILES: *NC(=O)CN1CCNCC1] | 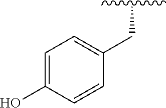 | 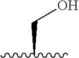 | 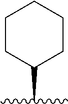 | H | 8.6 | 22.7 |
| 152C12 (36) | 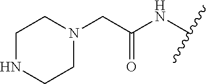 [SMILES: *NC(=O)CN1CCNCC1] | 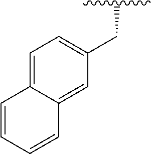 | 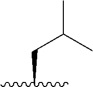 | 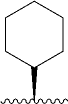 | Cl | — | 30.8 |
| 152D2 (37) | 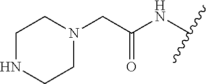 [SMILES: *NC(=O)CN1CCNCC1] | 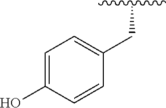 | 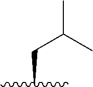 | 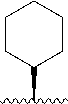 | Cl | 49 | 16.5 |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula (I): 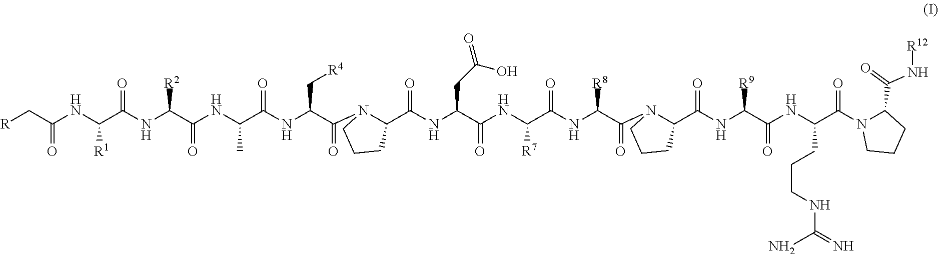
or a salt thereof, wherein:
each R is independently selected from the group consisting of morpholino, piperidino, and piperazine, that is optionally substituted with (C₁-C₆)alkyl;
each R¹ is benzyl, 3-indolylmethyl, 4-pyridinylmethyl, 1-naphthylmethyl, or 2-naphthylmethyl, which benzyl, 3-indolylmethyl, 4-pyridinylmethyl, 1-naphthylmethyl, and 2-naphthylmethyl is optionally substituted with one or more groups independently selected from hydroxy, amino, nitro, (C₁-C₆) alkoxy, and (C₁-C₆)alkyl;
each R² is independently selected from the group consisting of (C₁-C₆)alkyl that is optionally substituted with hydroxy;
each R⁴ is independently selected from the group consisting of biphenyl and phenoxyphenyl, which biphenyl and phenoxyphenyl is optionally substituted with one or more groups independently selected from halo, hydroxy, (C₁-C₆)alkyl, and (C₁-C₆) alkoxy, wherein each (C₁-C₆)alkyl and (C₁-C₆)alkoxy is optionally substituted with one or more groups independently selected from the group consisting of halo;
each R⁷ is (C₁-C₆)alkyl that is optionally substituted with hydroxy;
each R⁸ is independently selected from the group consisting of isopropyl and (C₃-C₆)cycloalkyl;
each R⁹ is independently selected from the group consisting of benzyl that is optionally substituted with one or more halo;
R¹² is H or is selected from the group consisting of: 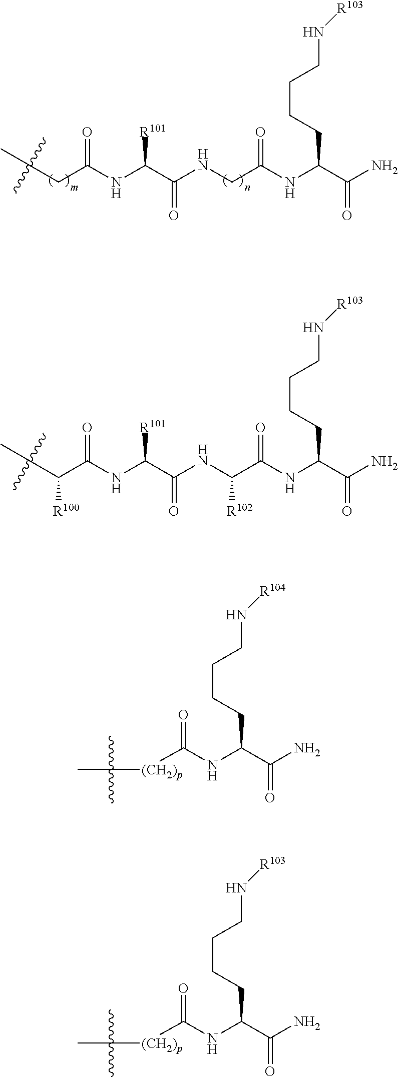 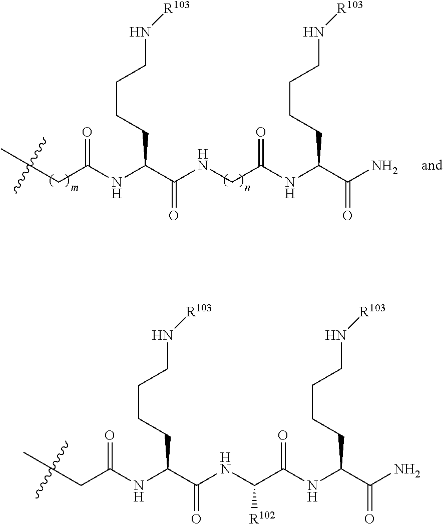
R¹⁰⁰ is H, (C₃-C₆)cycloalkyl, or (C₁-C₆)alkyl that is optionally substituted with hydroxy;
R¹⁰¹ is H, (C₃-C₆)cycloalkyl, or (C₁-C₆)alkyl that is optionally substituted with hydroxy;
R¹⁰² is H, (C₃-C₆)cycloalkyl, or (C₁-C₆)alkyl that is optionally substituted with hydroxy;
R¹⁰³ is -L¹-D;
D is the residue of a drug or the residue of a targeting agent;
p is 1, 2, or 3;
m is 1, 2, or 3;
n is 1, 2, or 3;
R¹⁰⁴ is: 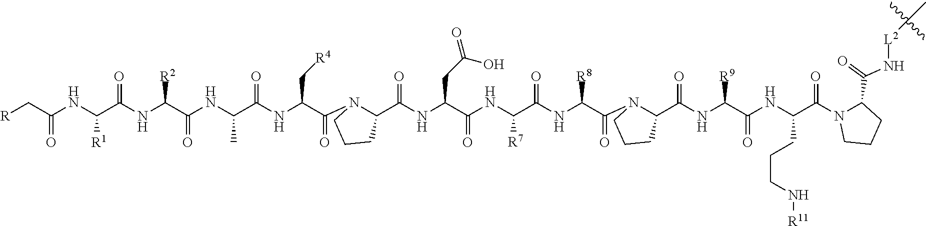
R¹¹ is C(═NH)NH₂;
L¹ is a linking group; and
L² is a linking group.

2. The compound or salt of claim 1, wherein;
each R¹ is benzyl, 1-naphthylmethyl, or 2-naphthylmethyl, which benzyl, 1-naphthylmethyl, and 2-naphthylmethyl is optionally substituted with one or more groups independently selected from hydroxy, amino, nitro, and (C₁-C₆)alkyl;
R¹² is H or is selected from the group consisting of: 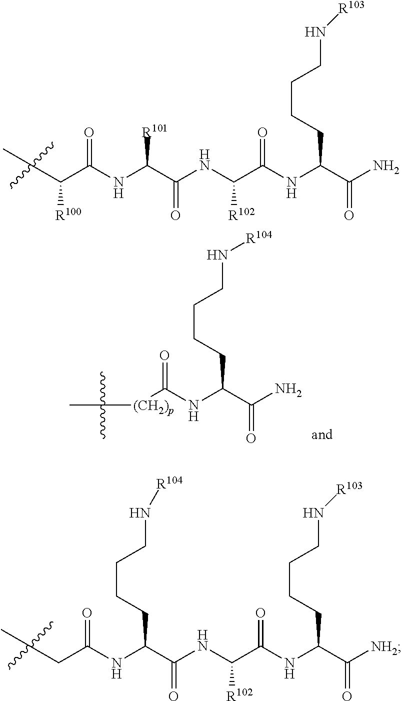
R¹⁰⁰ is H, (C₃-C₆)cycloalkyl, or (C₁-C₂)alkyl;
R¹⁰¹ is H, (C₃-C₆)cycloalkyl, or (C₁-C₂)alkyl; and
R¹⁰² is H, (C₃-C₆)cycloalkyl, or (C₁-C₂)alkyl.

3. (canceled)

4. The compound or salt of claim 1, wherein each R is piperazine, that is optionally substituted with (C₁-C₆)alkyl.

5-8. (canceled)

9. The compound or salt of claim 1, wherein each R¹ is 2-nitrobenzyl, 4-methylbenzyl, 4-hydroxybenzyl, or 4-aminobenzyl.

10. (canceled)

11. The compound or salt of claim 1, wherein each R² is isobutyl or hydroxymethyl.

12. (canceled)

13. (canceled)

14. The compound or salt of claim 1, wherein each R⁴ is independently selected from the group consisting of biphenyl, 2′-trifluoromethylbiphenyl, 2′-methylbiphenyl, 4′-chlorobiphenyl, 2′-methoxybiphenyl, 3′-methylbiphenyl, 2′-methyl-4′-methoxybiphenyl, phenoxyphenyl, and 4-(4-hydroxyphenyloxy)phenyl.

15. The compound or salt of claim 1, wherein each R⁸ is independently selected from the group consisting of isopropyl and (C₃-C₆)cycloalkyl.

16. (canceled)

17. The compound or salt of claim 1, wherein R¹² is H.

18. The compound or salt of claim 1, wherein R¹² is:

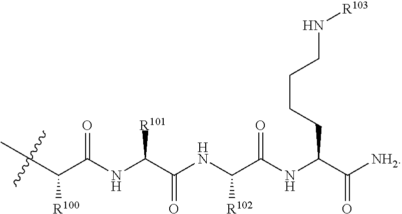

19. The compound or salt of claim 18, wherein R¹⁰⁰ is H, —CH₃, —C₂H₅, i-pr, cyclohexyl, or —CH₂OH.

20. The compound or salt of claim 18, wherein R¹⁰¹ is H, —CH₃, —C₂H₅, i-pr, cyclohexyl, or —CH₂OH.

21. The compound or salt of claim 18, wherein R¹⁰² is H, —CH₃, —C₂H₅, i-pr, cyclohexyl, or —CH₂OH.

22. The compound or salt of claim 1, wherein R¹² is:

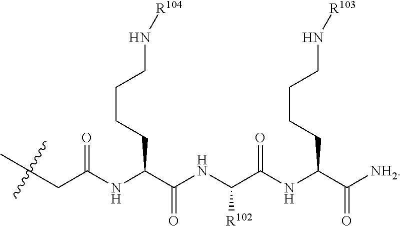

23-25. (canceled)

26. The compound or salt of claim 1, wherein L¹ is:

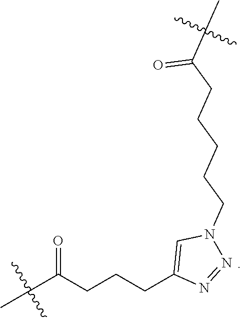 [SMILES: *C(=O)CCCCCn1cc(CCCC(*)=O)nn1]

27-28. (canceled)

29. The compound or salt of claim 1, wherein D is a residue of a taxane, including paclitaxel, docetaxel, or cabazitaxel.

30. The compound or salt of claim 1, wherein D is a residue of gemcitabine

31-36. (canceled)

37. The compound or salt of claim 1, wherein L² is —CH₂C(═O)—, —CH₂CH₂C(═O)—, —CH₂CH₂CH₂C(═O)—, —CH₂CH₂CH₂CH₂C(═O)—, or —CH₂CH₂CH₂CH₂CH₂C(═O)—.

38. A compound selected from the group consisting of: 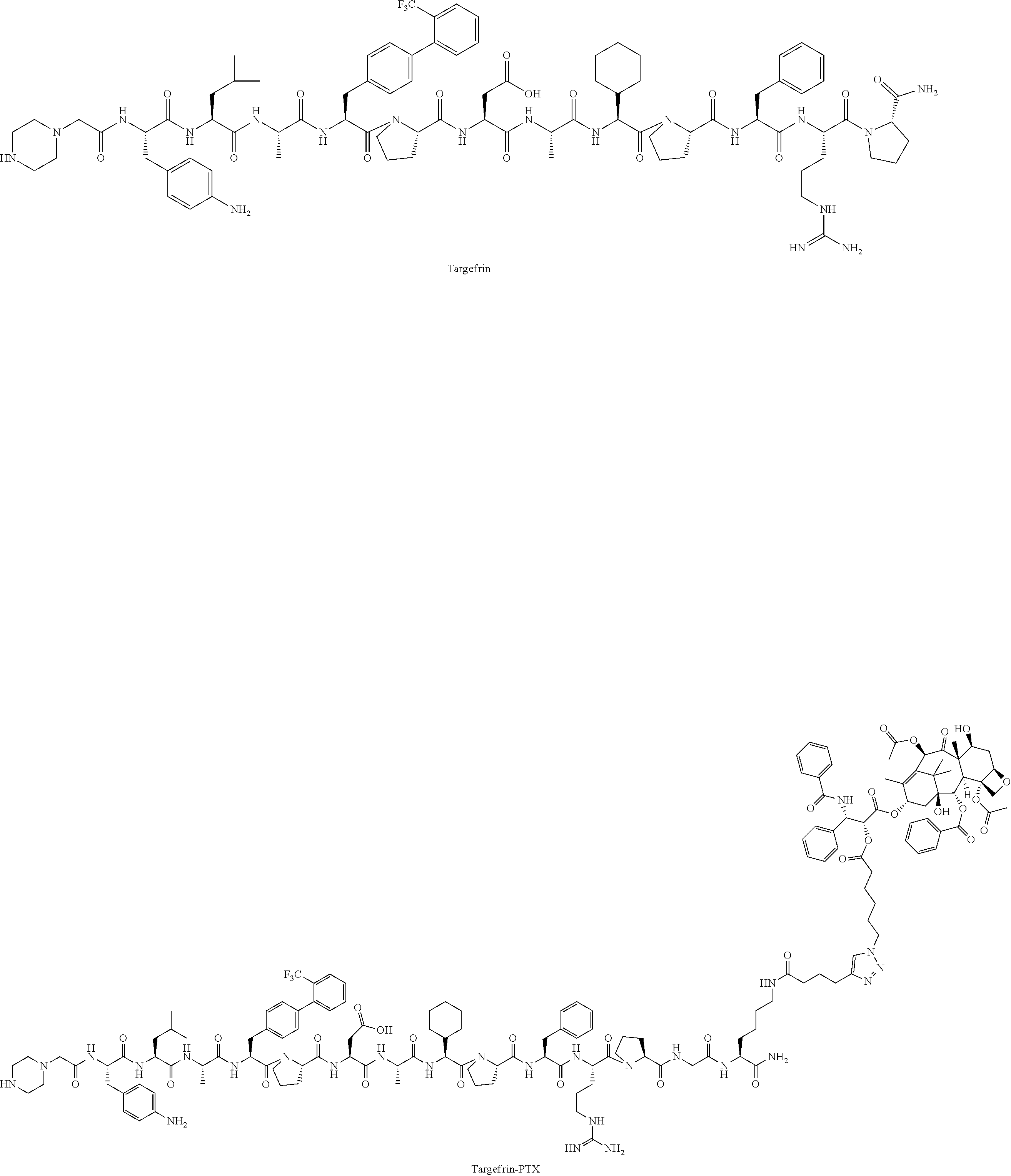 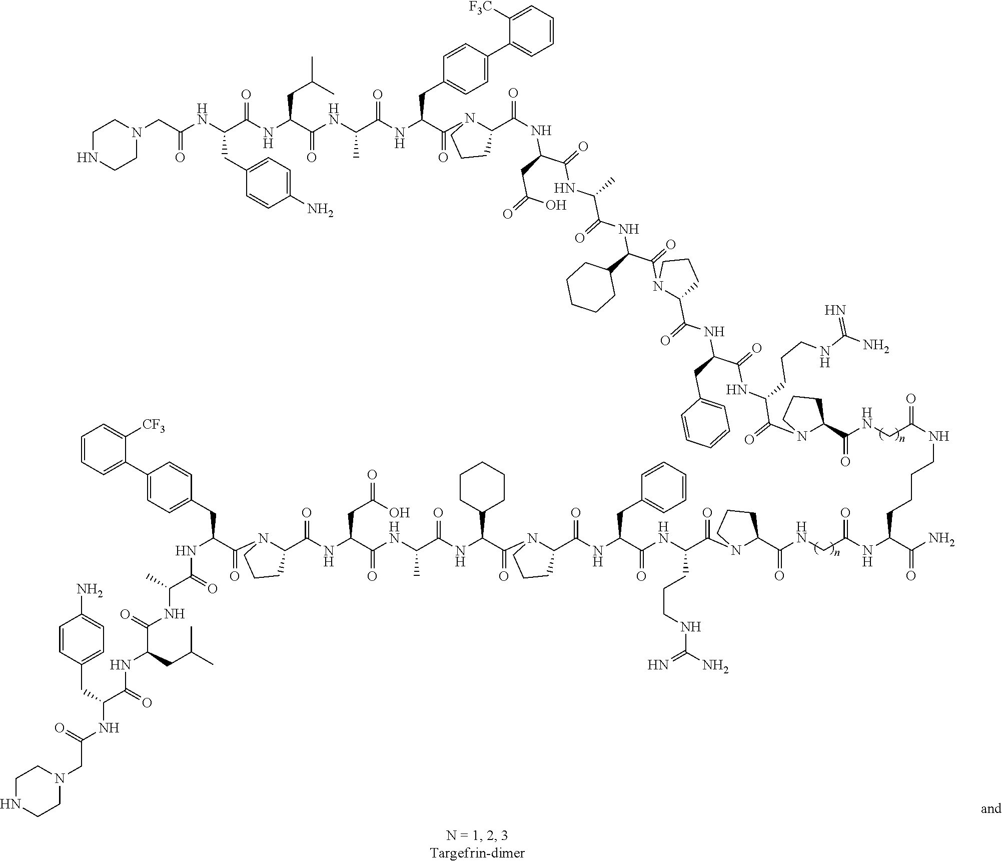 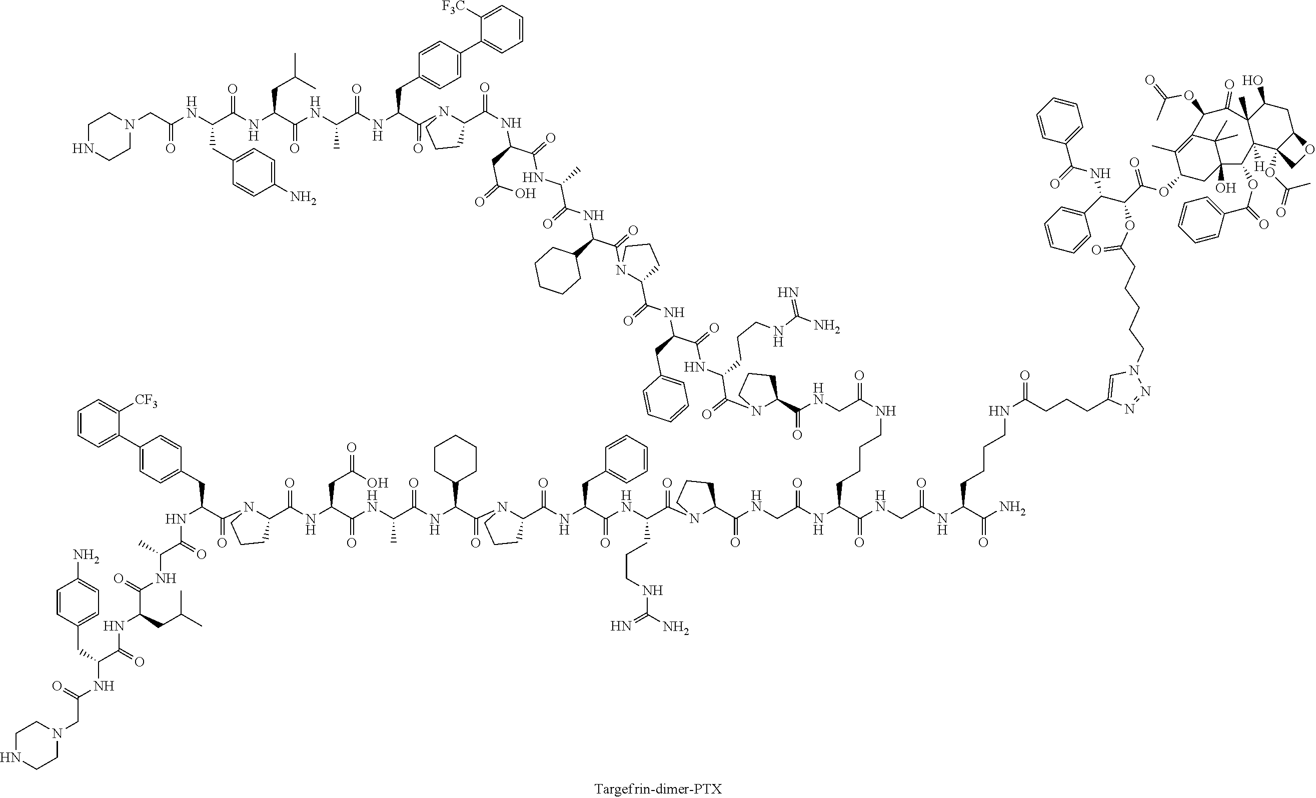
or a salt thereof.

39. (canceled)

40. A method for treating cancer in an animal comprising administering a compound or salt of claim 1 to the animal.

41-45. (canceled)
